(19) United States
(12) Patent Application Publication
(10) Pub. No.: US 2025/0332271 A1
(43) Pub. Date: Oct. 30, 2025

(54) PROGRAMMABLE SELECTIVE ACYLATION OF POLYOLS

(71) Applicant: Nanyang Technological University, Singapore (SG)

(72) Inventors: Yonggui CHI, Singapore (SG); Wenxin LYU, Singapore (SG)

(21) Appl. No.: 18/697,116

(22) PCT Filed: Oct. 5, 2022
(86) PCT No.: PCT/SG2022/050711

§ 371 Date: Mar. 29, 2024

(30) Foreign Application Priority Data
Oct. 5, 2021 (SG) 10202111103X

(51) Int. Cl. A61K 47/64; A61K 47/54; C07H 1/00; C07H 15/18; C07H 15/207; C07H 15/26; C07H 17/04

(52) U.S. Cl. CPC A61K 47/64; A61K 47/549; C07H 1/00; C07H 15/18; C07H 15/207; C07H 15/26; C07H 17/04

(57) ABSTRACT

Disclosed herein is a method to selectively acylate a polyol, the method comprising the steps of: (a) providing a mixture comprising a polyol, an acylation agent, a N-heterocyclic carbene (NHC) precursor, a base and a solvent; and (b) subjecting the mixture to an elevated temperature for a period of time to provide a selectively acylated polyol, optionally wherein the mixture further comprises boronic acid.

### FIELD OF INVENTION

The current invention relates to a method of selectively acylating polyols using a carbene catalyst, optionally in combination with a boronic acid.

### BACKGROUND

The listing or discussion of a prior-published document in this specification should not necessarily be taken as an acknowledgement that the document is part of the state of the art or is common general knowledge.

Saccharides are a major class of biomolecules involved in numerous biological activities. Saccharide derivatives and multi-hydroxyl group (polyol)-containing structures are also widely found in natural products and synthetic molecules with important functions (FIG. 1A). It has been proved that modulation of saccharides or saccharide segments can lead to therapeutic agents, such as vaccines and antibiotics, with billion-dollar commercial success. For example, bacterial-capsule polysaccharides attached to proteins have been a main choice for conjugated vaccines. Multiple saccharide-derived small molecules, such as Empagliflozin, are among the best-selling drugs. However, despite the enormous applications and potential, our understanding of saccharide-related biological processes and the development of saccharide-based pharmaceuticals remain challenging. A major obstacle lies in the lack of efficient chemical synthetic tools for access to saccharides and their derivatives. It is difficult to selectively functionalize the many hydroxyl (OH) groups present in saccharides because the reactivity differences of the various OH groups are very small.

Numerous approaches from the best chemists of many generations have been designed to achieve site-selective reactions on the different OH groups of saccharides and polyol molecules. The dominant approach involves elegantly designed orthogonal protection-deprotection chemistry through typically long-step operations, as demonstrated by many pioneers. Although improvements are being made in this protection-deprotection approach, new strategies with shorter steps that avoid (or minimize) conventional protection-deprotection operations have attracted intense attention for obvious reasons.

However, in these previous approaches, pre-protection of the C6- and/or C4-OH groups (of monosaccharides) is still necessary before selective reaction can be performed on the remaining OH units. The generality of monosaccharide partners is typically limited to those with certain structural requirements (such as the presence of cis-diols). Individual access to different sites (such as to C2-, C3-, and C6-sites individually) via each of these approaches is still difficult. Further breakthroughs in this arena of saccharide-selective reactions remain to emerge.

Therefore, there exists a need to discover new methods for selective acylation of saccharides (and polyols in general) and new saccharide-derived functional molecules.

### SUMMARY OF INVENTION

The current invention relates to a method of selectively acylating a polyol. Thus, in a first aspect of the invention, there is provided a method to selectively acylate a polyol, the method comprising the steps of:
(a) providing a mixture comprising a polyol, an acylation agent, a N-heterocyclic carbene (NHC) precursor, a base and a solvent; and
(b) subjecting the mixture to an elevated temperature for a period of time to provide a selectively acylated polyol, optionally wherein the mixture further comprises a boronic acid.

Embodiments of this invention will be discussed in the description below.

### DRAWINGS

FIG. 1 depicts the new strategy for selective acylation of saccharides and polyols.

FIG. 2 depicts the structures of (A)N-heterocyclic carbene (NHC) catalysts, and (B) boronic acids that are commercially available.

FIG. 3 depicts the method for determining the ratio and yield of the reaction by ¹H nuclear magnetic resonance (NMR) with the reaction of entry 2 in Table 1 as an example.

FIG. 4 depicts the typical conditions and a mechanistic pathway for selective C(3)-OH acylation of α-glucoside as a model saccharide.

FIG. 5 depicts the modular combinations of NHCs and boronic acids.

FIG. 6 depicts (A) the intermediate I and its reactivity in C2-OH selective acylation, (B)¹H NMR spectrum of the reaction mixture in (A), equation (eq) a, and (C)¹H NMR spectra of the reaction mixture in (A), eq b and c.

FIG. 7 depicts the plausible catalytic cycle in C2-OH selective acylation.

FIG. 8 depicts the intermediate I and its reactivity in C3-OH selective acylation.

FIG. 9 depicts the plausible catalytic cycle in C3-OH selective acylation.

FIG. 10 depicts the conditions for site-selective acylation of a model saccharide as mediated by NHC catalysts and boronic acids.

FIG. 11 depicts the scope of selective saccharide acylation using aldehydes as the acylation reagent. Yields are isolated yields of the major product; regioselectivity refers to C3/C2-O-acylate for 3-37 and 39-46, C3/C6-O-acylate for 38, and C6/C3-Oacylate for 47-60. Yields are isolated yields of two combined monoacylates for 36 and 40. R=p-Cl-Ph. Other possible acylation products were unobservable or minimal (less than 5%) via thin-layer chromatography (TLC) and NMR analysis of the crude reaction mixture.

FIG. 12 depicts the scope of selective saccharide acylation using carboxylic acids and esters as the acylation reagent. Yields are isolated yields of the major product; regioselectivity refers to C3/C2-O-acylate for 3, 62, and 39 (acylations on other OH groups are minimal). Yields are isolated yields of two combined acylates for 39 and 62. The regioselectivity value is not given when acylation on all other OH groups is trace. R=p-CI-Ph for products 3, 39, and 57.

FIG. 13 depicts the scope of aldehydes amenable to site-selective monoacylation, related to FIG. 11. Yields are isolated yields of C3-O-acylate, regioselectivity refers to C3/C2-O-acylate for 3-19.

FIG. 14 depicts the scope of saccharides amenable to C3-OH selective monoacylation, related to FIG. 11. Yields are isolated yields of C3-O-acylate, regioselectivity refers to C3/C2-O-acylate for 3-37, and C3/C6-O-acylate for 38. Yield of 36 is isolated yield of C3-O-acylate and C2-O-acylate.

FIG. 15 depicts the scope of saccharides amenable to C2-OH selective monoacylation, related to FIG. 11. Yields are isolated yields of C2-O-acylate, regioselectivity refers to C3/C2-O-acylate for 39-46. Yield of 40 is isolated yield of C3-O-acylate and C2-O-acylate.

FIG. 16 depicts the scope of saccharides amenable to C6-OH selective monoacylation, related to FIG. 11. Yields are isolated yields of C6-O-acylate, regioselectivity refers to C6/C3-O-acylate for 47-60.

FIG. 17 depicts the scope of carboxylic acids amenable to site-selective monoacylation, related to FIG. 12. Yields are isolated yields of monoacylate.

FIG. 18 depicts the scope of esters amenable to site-selective monoacylation, related to FIG. 12. Yields are isolated yields of monoacylate, regioselectivity refers to C3/C2-O-acylate for 3, 62, and 39. Yields of 62 and 39 are isolated yield of C3-O-acylate and C2-O-acylate.

FIG. 19 depicts the schematic representation of the non-covalent interactions (NCIs) involved in the key regioselective step for model reactions considered by density functional theory (DFT) calculations.

FIG. 20 depicts the model reactions for computational mechanistic studies.

FIG. 21 depicts the example intermediate structures for conformational sampling. Example rotational degrees of freedom about single bonds are shown in arrows.

FIG. 22 depicts the DFT optimized structures of the lowest energy conformers resulting from each of the reactions in FIG. 20. Relative Gibbs energy is taken with respect to the lowest energy conformer within each reaction.

FIG. 23 depicts the schematic representations of the possible interactions that will occur in the regioselective intermediates and similar transition states (TSs). The major C-OH acylation is in bold for each reaction.

FIG. 24 depicts the optimized TSs structures for the regio-determining transition states for the formation of C—O bond in the intermediate in Reaction 4. Key bond distances are given in A.

Relative activation barriers are given in kcal mol- and taken relative to the lowest activation barrier.

FIG. 25 depicts the concise synthesis of sophisticated functional molecules as enabled by our site-selective acylation strategy.

FIG. 26 depicts the comparison of our method with methods reported in literature.

### DESCRIPTION

It has been surprisingly found that some or all of the problems can be solved using the following method. Thus, in a first aspect of the invention, there is provided a method to selectively acylate a polyol, the method comprising the steps of:
(a) providing a mixture comprising a polyol, an acylation agent, a N-heterocyclic carbene (NHC) precursor, a base and a solvent; and
(b) subjecting the mixture to an elevated temperature for a period of time to provide a selectively acylated polyol, optionally wherein the mixture further comprises a boronic acid.

The method above is a programmable, multilayered selectivity amplification strategy enabled by N-heterocyclic carbene (NHC) catalysts (and in some cases boronic acids) for site-specific acylation of unprotected polyols (e.g. monosaccharides). The boronic acids, when used, may provide transient shielding on certain hydroxyl groups via dynamic covalent bonds to offer the first sets of selectivity controls. The NHC catalyst provides a layer of control by mediating selective acylation of the unshielded hydroxyl moieties. Multiple activating/deactivating forces brought by the boronic acids and NHC catalysts can be easily modulated. A large number of structurally diverse polyols (e.g. monosaccharides and their analogues) can be precisely reacted with different acylating reagents, offering quick access to sophisticated saccharide-derived products.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

The method disclosed herein is generic and can be used with molecules containing a broad range of functional groups without affecting the resulting product. Thus, the polyol is not particularly limited in its scope and a broad range of polyols may be used in the method disclosed herein. In embodiments of the invention, the polyol may be selected from a saccharide (e.g. a mono- or di-saccharide) and a sugar alcohol.

Examples of saccharides and sugar alcohols that may be mentioned herein include, but are not limited to:
glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol,

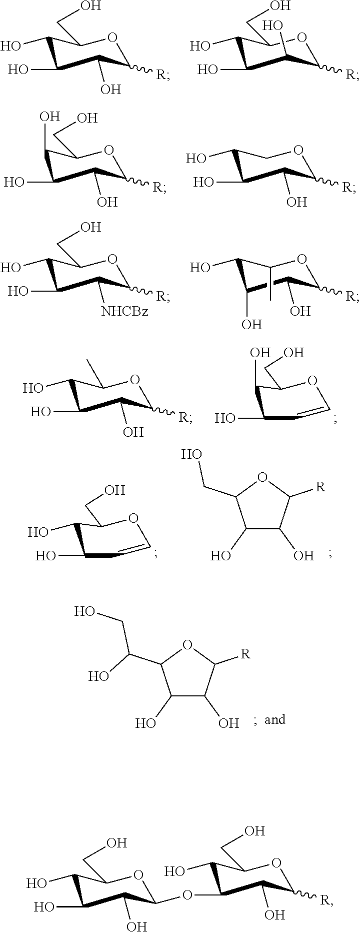

where R is any suitable moiety.

For example, R may be selected from
(i) H;
(ii) halo;
(iii) alkyl, alkenyl, alkynyl, cycloalkyl, cycloalkenyl, which five groups are unsubstituted or substituted by one or more substituents selected from halo, nitro, CN, alkyl, alkenyl, alkynyl, cycloalkyl, aryl, heterocyclyl, OR^{1a}, S(O)ₙR^{1b}, S(O)₂N(R^{1c})(R^{1d}), N(R^{1e})S(O)₂R^{1f}, N(R^{1g})(R^{1h})
where the alkyl, alkenyl and alkynyl groups are unsubstituted or substituted by one or more substituents selected from OH, ═O, halo, alkyl and alkoxy, and
where the cycloalkyl or cycloalkenyl groups may additionally be substituted by ═O;
(iv) S(O)ₚR¹ⁱ
(v) S(O)₂N(R^{1j})(R^{1k}),
(vi) OR^{1l},
(vii) N(R^{1m})(R¹ⁿ),
(viii) N(R^{1o})S(O)₂R^{1p},
(ix) aryl; or
(x) heterocyclyl, where
R^{1a} to R^{1p} independently represent, at each occurrence H or C₁₋₄ alkyl, which latter group is unsubstituted or substituted by one or more substituents selected from halo, OH and NH₂; n and p are independently 0, 1 or 2.

The term “halo”, when used herein, includes references to fluoro, chloro, bromo and iodo.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, acyclic or cyclic, saturated or unsaturated (so forming, for example, an alkenyl or alkynyl) hydrocarbyl radical, which may be unsubstituted or substituted (with, for example, one or more halo atoms). Where the term “alkyl” refers to an acyclic group, it is preferably C₁₋₁₀ alkyl and, more preferably, C1.e alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

Unless otherwise specified herein, a “heterocyclyl” or a “heterocyclic ring system” may be a 4-to 14-membered, such as a 5- to 10-membered (e.g. 6- to 10-membered), heterocyclic group that may be aromatic, fully saturated or partially unsaturated, and which contains one or more heteroatoms selected from O, S and N, which heterocyclic group may comprise one or two rings. Examples of heterocyclic ring systems that may be mentioned herein include, but are not limited to azetidinyl, dihydrofuranyl (e.g. 2,3-dihydrofuranyl, 2,5-dihydrofuranyl), dihydropyranyl (e.g. 3,4-dihydropyranyl, 3,6-dihydropyranyl), 4,5-dihydro-1H-maleimido, dioxanyl, dioxolanyl, furanyl, furazanyl, hexahydropyrimidinyl, hydantoinyl, imidazolyl, isothiaziolyl, isoxazolidinyl, isoxazolyl, morpholinyl, 1,2- or 1,3-oxazinanyl, oxazolidinyl, oxazolyl, piperidinyl, piperazinyl, pyranyl, pyrazinyl, pyridazinyl, pyrazolyl, pyridinyl, pyrimidinyl, pyrrolinyl (e.g. 3-pyrrolinyl), pyrrolyl, pyrrolidinyl, pyrrolidinonyl, 3-sulfolenyl, sulfolanyl, tetrahydrofuranyl, tetrahydropyranyl, tetrahydropyridinyl (e.g. 3,4,5,6-tetrahydropyridinyl), 1,2,3,4-tetrahydropyrimidinyl, 3,4,5,6-tetrahydropyrimidinyl, tetrahydrothiophenyl, tetramethylenesulfoxide, tetrazolyl, thiadiazolyl, thiazolyl, thiazolidinyl, thienyl, thiophenethyl, triazolyl and triazinanyl.

When the heterocyclic ring system is aromatic, it may be referred to as a heteroaryl ring system. The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

Unless otherwise specified herein, a “carbocyclic ring system” may be a 4- to 14-membered, such as a 5- to 10-membered (e.g. 6- to 10-membered, such as a 6-membered or 10-membered), carbocyclic group that may be aromatic, fully saturated or partially unsaturated, which carbocyclic group may comprise one or two rings. Examples of carbocyclic ring systems that may be mentioned herein include, but are not limited to cyclobutyl, cyclopentyl, cyclohexyl, cyclooctyl, phenyl, naphthyl, decalinyl, tetralinyl, bicyclo[4.2.0]octanyl, and 2,3,3a,4,5,6,7,7a-octahydro-1H-indanyl. Particularly preferred carbocyclic groups include phenyl, cyclohexyl and naphthyl.

In more particular embodiments that may be mentioned herein, the polyol may be selected from the group consisting of:

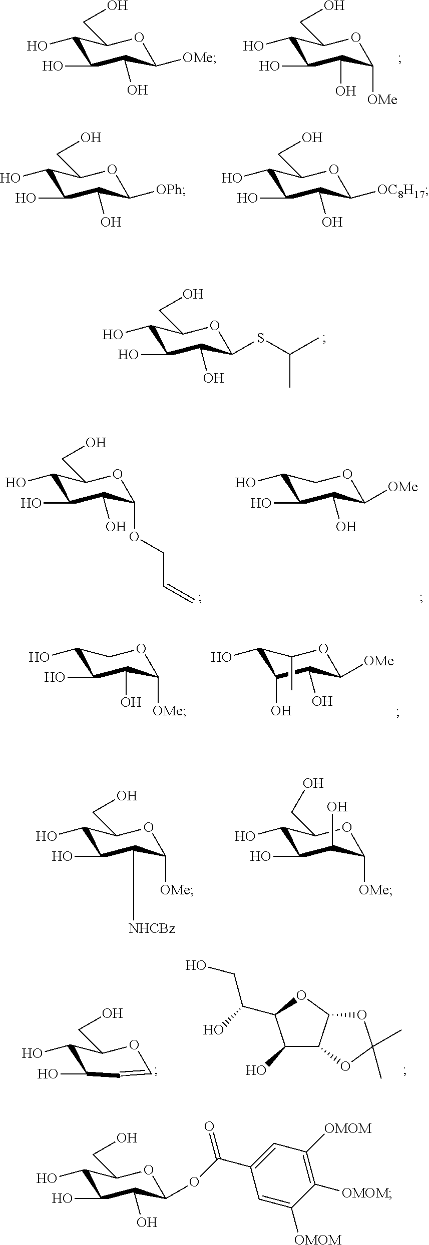

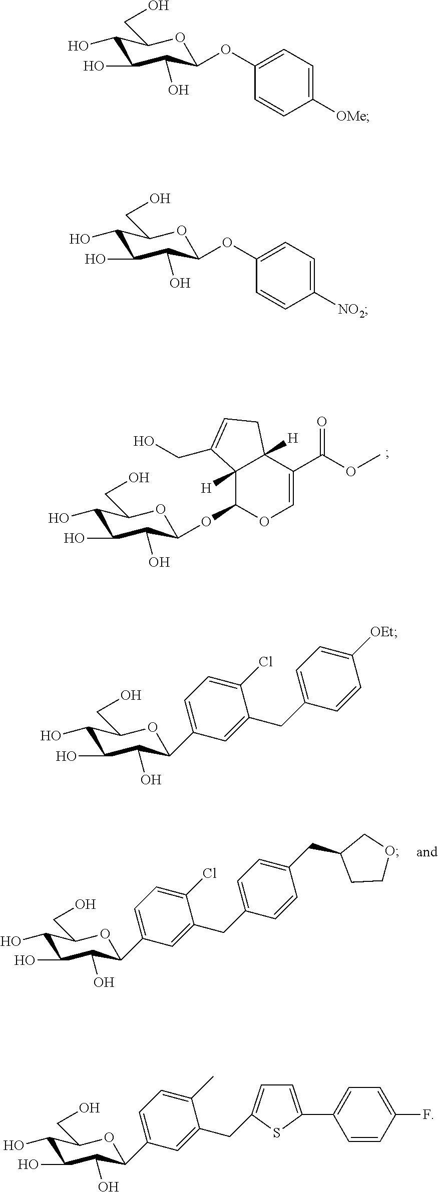

As noted above, the method disclosed herein is not particularly limited in the types of reagents that may be used. Therefore, any suitable acylation agent may be used. For example, the acylation agent may be selected from:

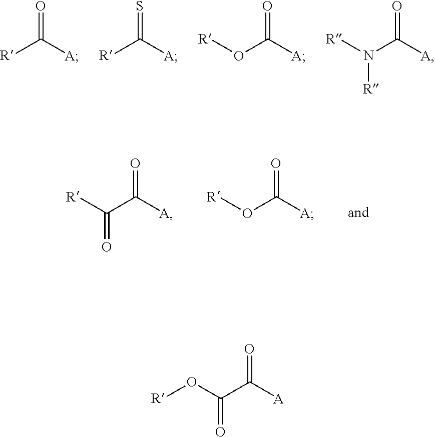

where:

A represents a moiety which forms a functional group suitable to react with a hydroxyl group to form an ester; and

R′ and R″ independently represent H or an organic moiety.

The identity of R′ and R″ is not particularly limited and virtually any organic moiety may be used, either in its unprotected form or with protecting groups. The protection and deprotection of functional groups may take place before or after a reaction. As will be appreciated, an advantage of the current methodology is that the polyol hydroxyl groups do not need to be protected to effect the desired acylation.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

As used herein, the term “functional groups” means, in the case of unprotected functional groups, hydroxy-, thiolo-, amino-, carboxylic acid and, in the case of protected functional groups, lower alkoxy, N-, O-, S-acetyl, and carboxylic acid ester.

In embodiments of the invention that may be mentioned herein, R′ may be selected from: (bi)alkyl, alkenyl, alkynyl, cycloalkyl, cycloalkenyl, which five groups are unsubstituted or substituted by one or more substituents selected from halo, nitro, CN, alkyl, alkenyl, alkynyl, cycloalkyl, aryl, heterocyclyl, OR^{3a}, S(O)ₙR^{3b}, S(O)₂N(R^{3c})(R^{3d}), N(R^{3e})S(O)₂R^{3f}, N(R^{3g})(R^{3h})
where the alkyl alkenyl and alkynyl groups are unsubstituted or substituted by one or more substituents selected from OH, ═O, halo, alkyl and alkoxy, and
where the cycloalkyl or cycloalkenyl groups may additionally be substituted by ═O;
(bii) N(R^{3l})(R^{3m}),
(biii) N(R³ⁿ)S(O)₂R^{3o},
(biv) aryl; or
(bv) heterocyclyl, where

R^{3a} to R^{3o} independently represent, at each occurrence H or C₁₋₄ alkyl, which latter group is unsubstituted or substituted by one or more substituents selected from halo, OH and NH₂; n is 1 or 2.

In embodiments of the invention that may be mentioned herein, R″ may be selected from: (ci) alkyl, alkenyl, alkynyl, cycloalkyl, cycloalkenyl, which five groups are unsubstituted or substituted by one or more substituents selected from halo, nitro, CN, alkyl, alkenyl, alkynyl, cycloalkyl, aryl, heterocyclyl, OR^{4a}, S(O)ₙR^{4b}, S(O)₂N(R^{4c})(R^{4d}), N(R^{4e})S(O)₂R^{4f}, N(R^{4g})(R^{4h})
where the alkyl, alkenyl and alkynyl groups are unsubstituted or substituted by one or more substituents selected from OH, ═O, halo, alkyl and alkoxy, and
where the cycloalkyl or cycloalkenyl groups may additionally be substituted by ═O;
(cii) aryl; or
(ciii) heterocyclyl, where

R^{4a} to R^{4h} independently represent, at each occurrence H or C₁₋₄ alkyl, which latter group is unsubstituted or substituted by one or more substituents selected from halo, OH and NH₂;
n is 1 or 2.

In embodiments that may be mentioned herein, A may represent H, OH, halo, OR^{2a}, aryl and heterocyclyl, where R^{2a} represents alkyl or aryl.

In particular embodiments of the method that may be mentioned herein:
(ai) when A is H, the mixture may further comprise an oxidising agent, optionally wherein the oxidising agent is selected from MnO₂, PIDA, IBX or, more particularly, 3,3,5,5-Tetra-tert-butyldiphenoquinone (DQ); or
(aii) when A is OH, the mixture may further comprise a coupling agent, selected from one or more of the group consisting of 1-ethyl-3-(3-dimethylaminopropyl)carbodiimide (EDCl), hexafluorophosphate Azabenzotriazole Tetramethyl Uronium (HATU), 1-hydroxybenzotriazole (HOBT), N,N′-diisopropylcarbodiimide (DIC), (2-(1H-benzotriazol-1-yl)-1,1,3,3-tetramethyluronium hexafluorophosphate (HBTU) and, more particularly, N,N′-dicyclohexylcarbodiimide (DCC).

In yet more particular embodiments of the invention, the acylation agent may be selected from:

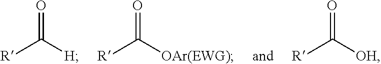

where R′ is as described above and Ar(EWG) represents an aryl group substituted by at least one electron withdrawing group.

In particular embodiments that may be mentioned herein, the acylation agent may be selected from:

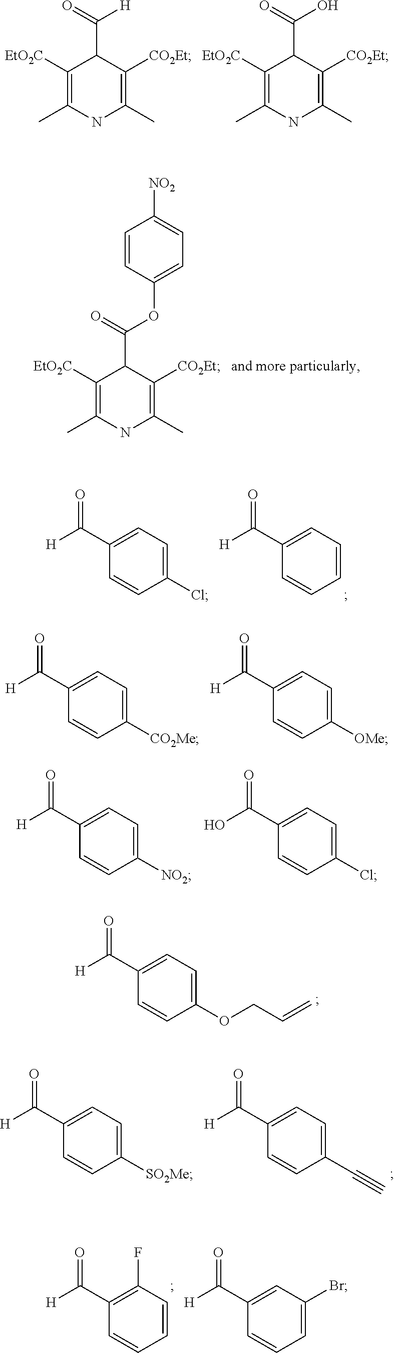

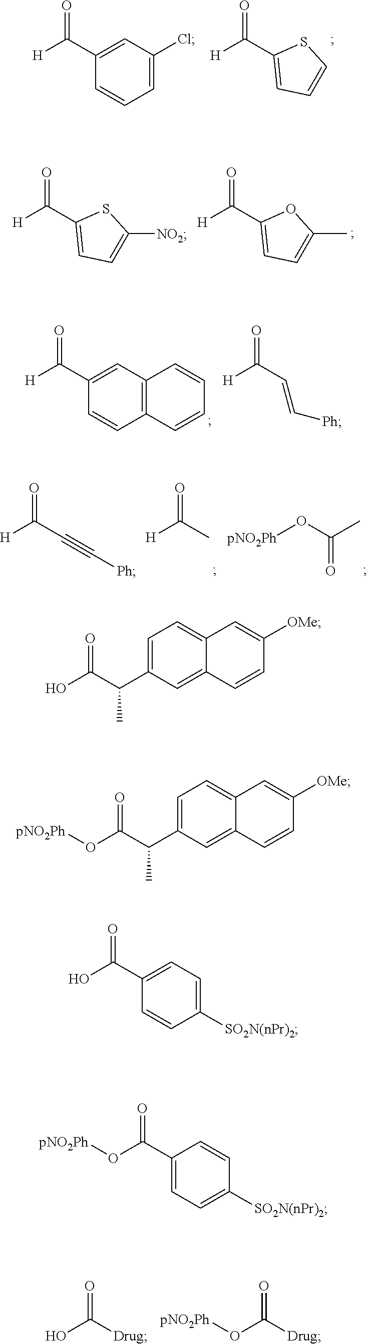

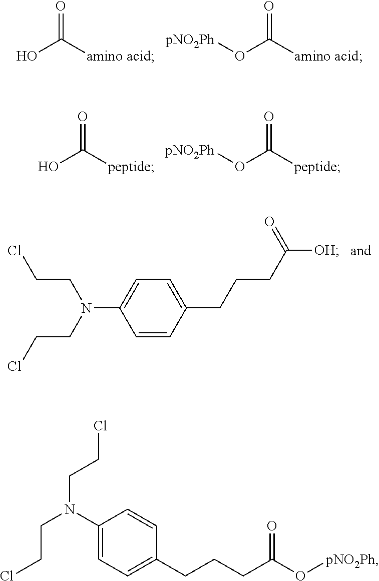

where:

Drug is any drug moiety (e.g. artesunate, dehydrocholic acid, (R)-hydratropic acid, ibuprofen, flurbiprofen, ketoprofen, nateglinide, paclitaxel) that is linked directly to the rest of the molecule or is linked via a suitable linking moiety to the rest of the molecule;
amino acid is any amino acid (e.g. a protected amino acid, such as Cbz-Phe-OH, CBz-Leu-OH, CBz-Met-OH, CBz-Val-OH, Boc-Ser(Bzl)-OH, Boc-Thr(Bzl)-OH, Boc-Trp(Boc)-OH, and Cbz-Lys(Boc)-OH); and
peptide is any peptide (e.g. aspartame).

Any N-heterocyclic carbene precursor may be used herein. Examples of suitable NHC precursors include, but are not limited to, a pyrrolidine-based triazolium salt, a morpholine-based triazolium salt, an aminoindane-based triazolium salt, an acyclic triazolium salt, an imidazole-based heteroazolium salt, an oxazolidine-based heteroazolium salt, an imidazoline-based heteroazolium salt, or a thiazole-based heteroazolium salt. Particular examples that may be mentioned herein include, but are not limited to:

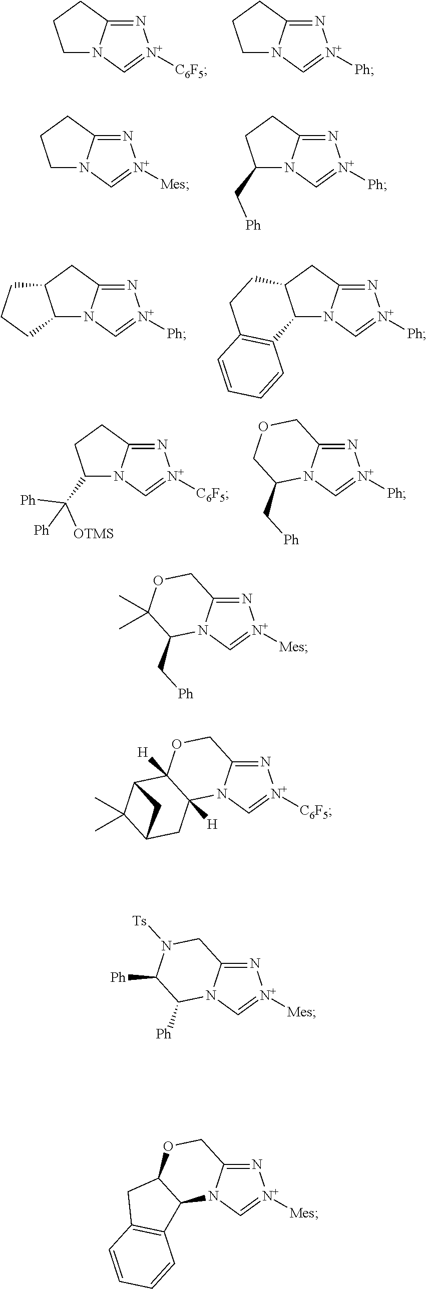

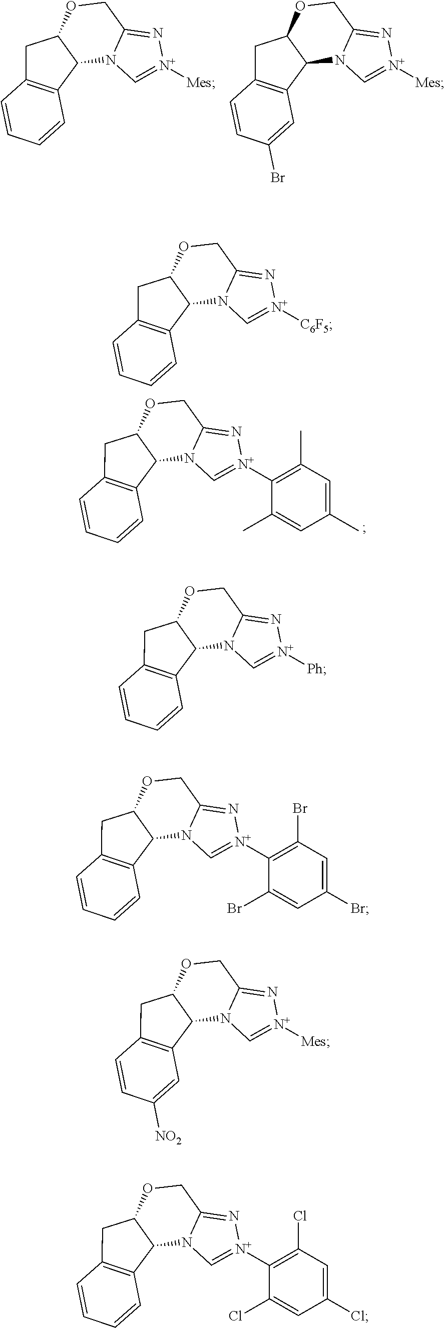

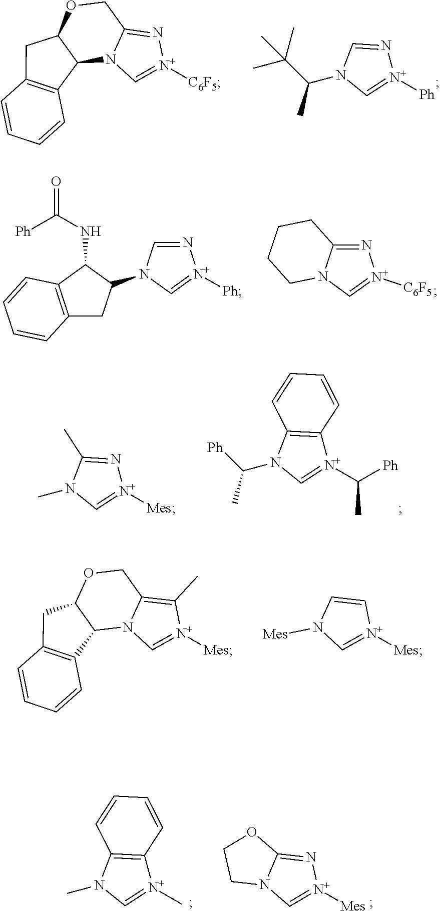

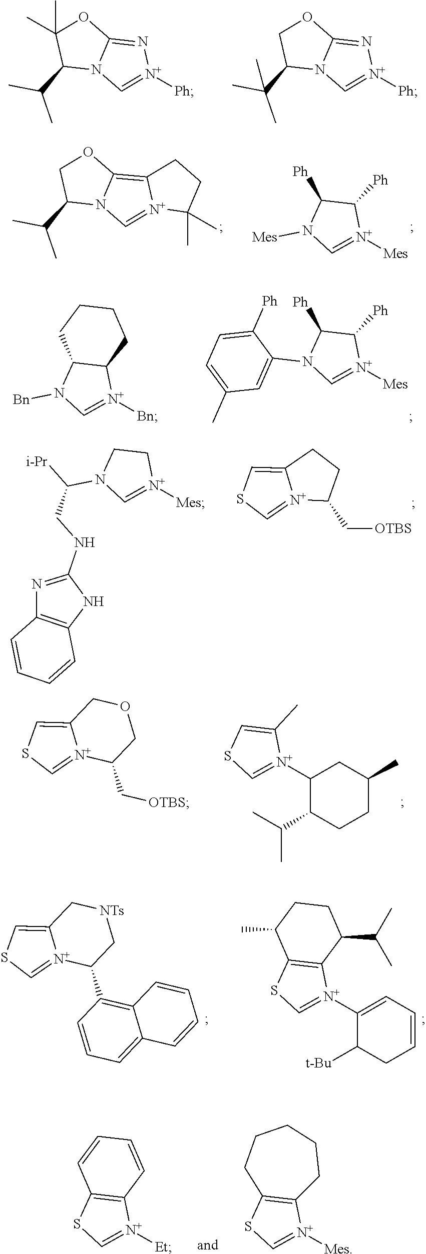

While not essential for the selective acylation to occur, it may be beneficial to make use of a boronic acid to enhance the selectivity in certain cases. Again, any suitable boronic acid may be used in the method disclosed herein when it is present as part of the reaction mixture. In embodiments of the invention that may be mentioned herein, the boronic acid may be selected from:

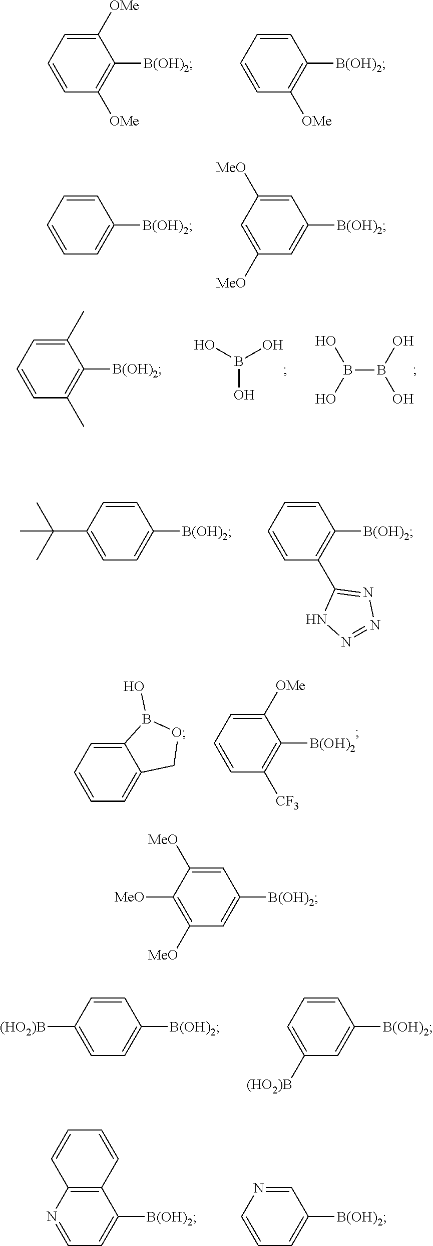

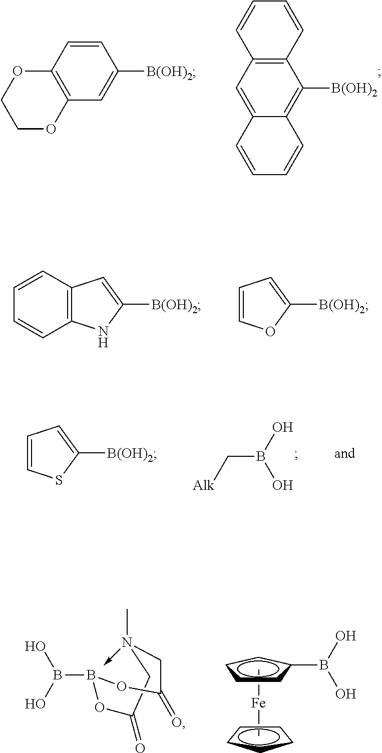

where Alk represents an alkyl group.

Any suitable base may be used in the method. Examples of suitable bases include, but are not limited to 1,4-diazabicyclo[2.2.2]octane (DABCO), K₂CO₃, Li₂CO₃, N,N-diisopropylethylamine (DIPEA), 1,8-diazabicyclo[5.4.0]undec-7-ene (DBU), triethylamine (NEt₃), and NaOAc.

Any suitable solvent may be used herein. Examples of suitable solvents include, but are not limited to tetrahydrofuran (THF), dichloromethane (DCM), acetonitrile (MeCN), toluene, dimethylformamide (DMF), dimethylsulfoxide (DMSO), ethyl acetate (EtOAc), acetone, or 1,4-dioxane.

As noted hereinbefore, the method may use an elevated temperature. That is, a temperature greater than the temperature of the ambient environment that the reaction is conducted in. This elevated temperature may be selected to be below the boiling point of the solvent selected or it may be at (or above) the boiling point of the selected solvent (in which case, the reaction may make use of a refluxing system). Alternatively, the elevated temperature may be significantly above the boiling point of the solvent (e.g. when the reaction is conducted in a sealed vessel). For example, the elevated temperature may be from 30 to 100° C., such as from 40 to 75° C., such as from 45 to 55° C., such as about 50° C.

As will be appreciated, for any given polyol, it will be required to make a selection of an acylation reagent, a NHC precursor, a base and a solvent, and possibly a boronic acid in order to obtain the desired selectivity. As discussed below, taking the tools disclosed herein, it is possible to optimise the desired selective acylation(s) using a few reactions to work out the most promising conditions for the polyol in question. Further details of this optimisation strategy are discussed in the examples section below.

The methods disclosed herein may allow for the selective acylation of a C(2)-, C(3)-, or (C6)-OH group on a monosaccharide or on a polyol, which might not otherwise be achievable without extensive use of protecting groups on the hydroxyl groups that are not desired to be acylated.

With D-glucose (primary alcohol group unprotected) as a model example, the use of a boronic acid additive can selectively shield the two hydroxyl groups at C4- and C6-carbons by forming a six-membered boronic ester with labile boron-oxygen bonds. This dynamic boronic ester formation temporarily protects these two hydroxyl groups from further reactions, providing the first layer of selectivity control. The introduction of boronic acid additives may also simultaneously accelerate reactions of certain hydroxyl groups, offering a second layer of selectivity control. In the same reaction solution, a N-heterocyclic carbene (NHC, or abbreviated as carbene) organic catalyst is introduced to provide a further layer of site selectivity control. Multiple parameters involving stereo electronic effects and covalent/non-covalent interactions brought by the boronic acids and NHC catalysts can be readily modulated. With this approach, through appropriate combined choices of boronic acids and/or NHCs, acyl group can be site-specifically installed on C(2)-OH, C(3)-OH, or C(6)-OH of D-glucose. This strategy can be easily tuned for site-specific acylation of various monosaccharides and their analogs by varying the structures of boronic acids and/or NHC catalysts. Sophisticated molecules (such as natural products) containing saccharide fragments can also undergo selective acylation reactions with different carboxylic acids and derivatives, including those with commercial applications as medicines (such as Artesunate and Dehydrocholic acid). Applications of our selective acylation strategy can allow for concise synthesis of saccharide-derived products such as (R)-Punicafolin and disaccharide laminaribiose with important bioactivities.

With the method outlined herein, the C(2)-, C(3)-, and (C6)-OH groups of various monosaccharides and their analogues can be selectively acylated. Aldehydes, carboxylic acids, and carboxylic esters can all be used as the acylation reagents. As demonstrated in the examples, carboxylic acid/saccharide-containing pharmaceuticals, peptides, natural products and other functional molecules can be site-selectively modified using this methodology. Application of this site-selective reaction can allow for concise and scalable access to complicated molecules such as disaccharides and bioactive natural products.

Without wishing to be bound by theory, it is believed that the selectivity was achieved by NHC organic catalysts alone or in combination with boronic acids. The synergistic activation and deactivation effects brought by the NHC and boronic acid dramatically amplify the reactivity difference of the multiple otherwise similar hydroxyl groups on polyols (e.g. saccharides). Such synergistic effects can also invert the initial reactivity preference of these hydroxyl moieties, offering selectivity patterns that are not available with previous strategies. As such, the C(2)-, C(3)-, and (C6)-OH groups of various monosaccharides and their analogues can be selectively acylated. Aldehydes, carboxylic acids, and carboxylic esters can all be used as the acylation reagents. We have also demonstrated that carboxylic acid/saccharide-containing pharmaceuticals, peptides, natural products and other functional molecules can be site-selectively modified using this methodology. Application of this site-selective reaction can allow for concise and scalable access to complex molecules such as disaccharides and bioactive natural products.

Further aspects and embodiments of the invention will now be discussed by reference to the following non-limiting examples.

### EXAMPLES

### Materials

Monosaccharides and boronic acids were purchased from Sigma-Aldrich, Alfa-Aesar, Titan. 3,3,5,5-Tetra-tert-butyldiphenoquinone (DQ) was used after purification in a pure state. Anhydrous CH₃CN, dichloromethane (DCM), tetrahydrofuran (THF) and dimethyl sulfoxide (DMSO) were purchased from Acros and stored under argon. Commercially available chemicals were obtained from commercial suppliers and used without further purification unless otherwise stated.

Analytical techniques
NMR spectroscopy

Proton (¹H) and carbon (¹³C) NMR were recorded with 400 MHz and 101 MHz NMR spectrometers, respectively. The following abbreviations are used for the multiplicities: s: singlet; d: doublet; t: triplet; q: quartet; m: multiplet; and brs: broad singlet; for proton spectra. Coupling constants (J) are reported in Hertz (Hz).

High-resolution mass spectra (HRMS)

HRMS were recorded on a BRUKER VPEXII spectrometer with ESI mode unless otherwise stated.

Thin layer chromatography (TLC)

Analytical TLC was performed on Polygram SIL G/UV254 plates. Visualization was accomplished with short wave UV light, or KMnO₄ staining solutions followed by heating.

Flash column chromatography

Flash column chromatography was performed using silica gel (200-300 mesh) with solvents distilled prior to use.

General procedure for the preparation of NHC Pre-catalysts

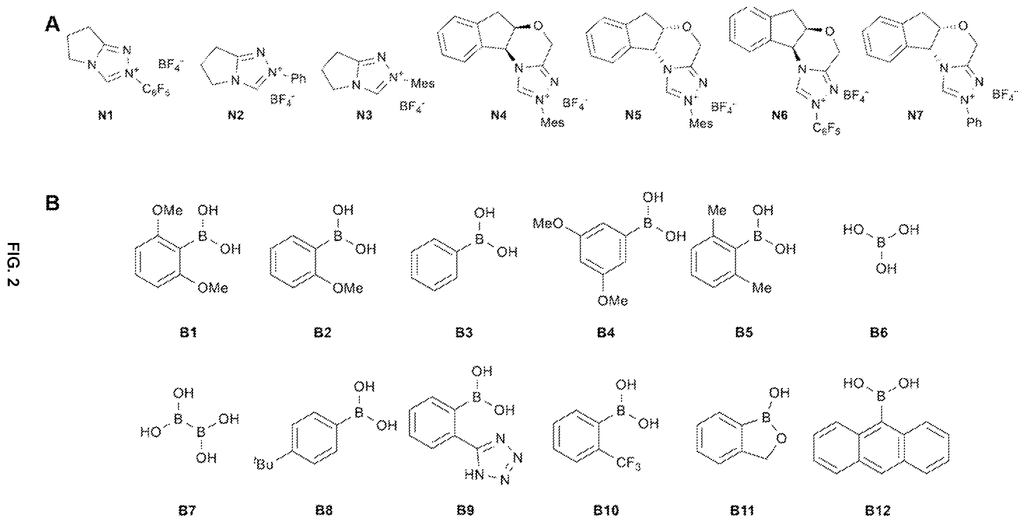

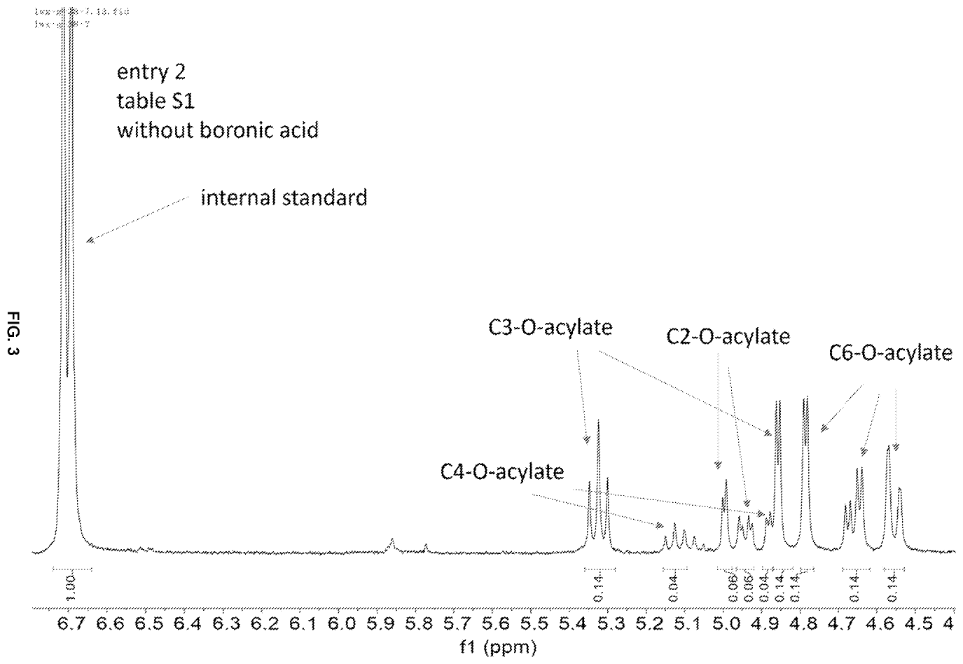

NHC pre-catalysts N1-N7 (FIG. 2A) were prepared according to the reference (Kerr, M. S., de Alaniz, J. R. & Rovis, T. J., Org. Chem. 2005, 70, 5725-5728; and Wu, Z. & Wang, J., ACS Catal. 2017, 7, 7647-7652).

General procedure for determining the ratio and yield of the reaction by ¹H NMR

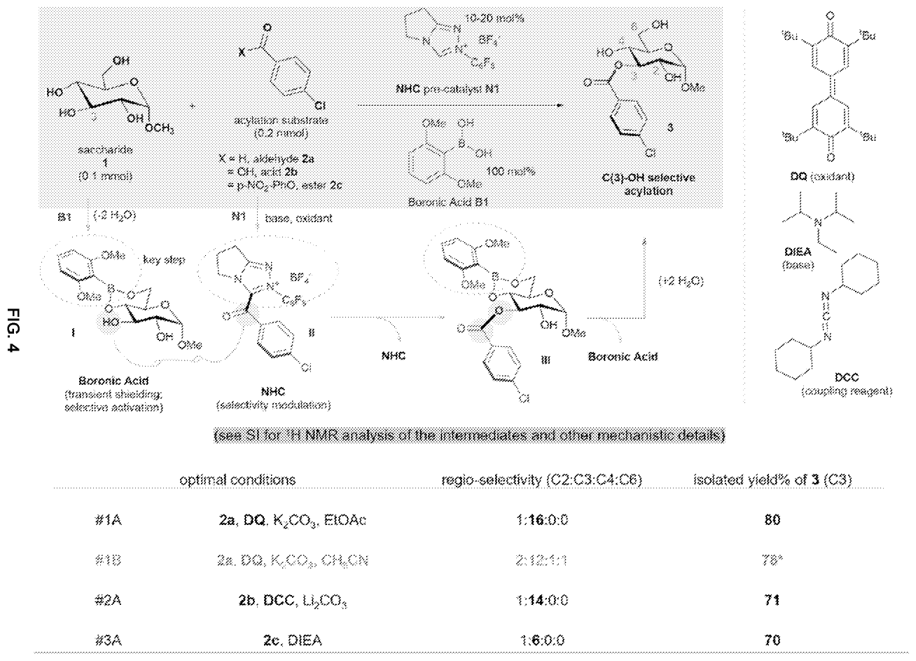

The reaction mixture was purified by flash column chromatography on silica with an appropriate solvent (ethyl acetate/hexane 1:1 to 1:0 v/v) to afford the mixture acylates. Paraiodoanisole (0.05 mmol) was used as the internal standard to measure the NMR yield. An example of measuring the NMR yield is depicted in FIG. 3. ¹H NMR of C4-O-acylate was referred to (Li, Y. & Kluger, R., J. Org. Chem. 2018, 83, 7360-7365).

### Example 1. A programmable strategy mediated by multiple driving forces for site-selective acylation of unprotected monoglycosides, their analogs, and their derivatives

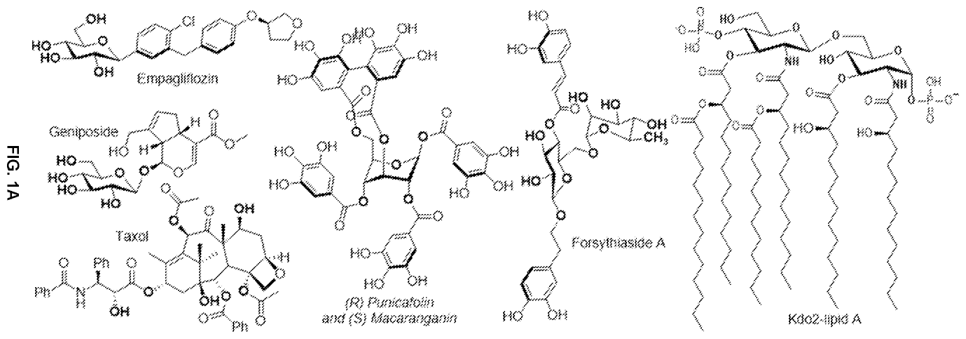

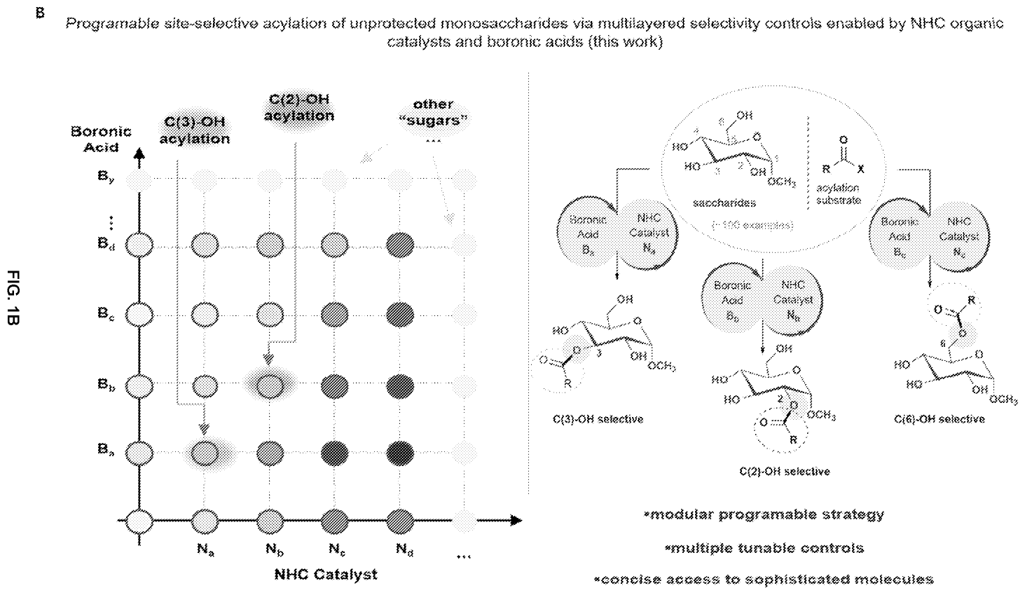

Here, we disclose a programmable strategy mediated by multiple driving forces for site-selective acylation of unprotected monoglycosides, their analogues, and their derivatives (FIG. 1B). We break down the challenging selectivity problem into a few smaller issues, each of which can be addressed by different cooperative catalysts and additives. With D-glucoside (primary alcohol group unprotected) as a model example, the use of boronic acid additive can selectively shield the two OH groups at the C4- and C6-carbons by forming a six-membered boronic ester with labile boron-oxygen bonds. This dynamic boronic ester formation temporarily protects these two OH groups from further reactions, providing the first layer of selectivity control. The introduction of boronic acid additives can also simultaneously accelerate reactions of certain OH groups, offering a second layer of selectivity control. In the same reaction solution, an NHC organic catalyst is introduced to provide a further layer of site-selectivity control. Multiple parameters involving stereo-electronic effects and covalent and/or NCIs brought by the boronic acids and NHC catalysts can be readily modulated. With our approach, through appropriate combined choices of boronic acids and/or NHCs, the acyl group can be site-selectively installed on the C(2)-OH, C(3)-OH, or C(6)-OH of D-glucoside. As demonstrated in the following examples, our strategy can be easily tuned for site-specific acylation of various monosaccharides and their analogs by varying the structures of boronic acids and/or NHC catalysts (as illustrated in the left graph of FIG. 1B). Sophisticated molecules (such as natural products) containing saccharide fragments can also undergo selective acylation reactions with different carboxylic acids and derivatives, including those with commercial applications as medicines (such as artesunate and dehydrocholic acid). Applications of our selective acylation strategy can allow for concise synthesis of saccharide-derived products, such as (R)-punicafolin, (S)-macaranganin, and disaccharide laminaribiose, with important bioactivities.

### Example 2. Optimization of the site-selective acylation of unprotected monoglycosides, their analogs, and their derivatives

Selective acylation with aldehydes as acylation reagents (General Procedure A)

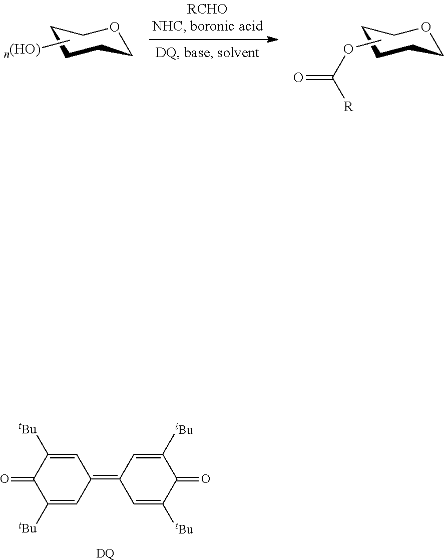

Monosaccharide (0.1 mmol, 1.0 equiv), aldehyde (0.2 mmol, 2.0 equiv), NHC catalyst (10 mol %), boronic acid (1.0-1.5 equiv), DQ (1.0-1.5 equiv), and base (0.02 mmol, 0.2 equiv) were added to a 4 mL screwtop test tube. Then, solvent (2 mL) was added to the mixture. The reaction mixture was allowed to stir vigorously at 50° C. for 1-12 h under a N₂ atmosphere. After cooling to room temperature, the reaction mixture was directly purified by flash column chromatography on silica with an appropriate solvent (EtOAc/hexane 1:5 to 5:1 v/v) to afford the pure product. Extraction with EtOAc/saturated aqueous NaHCO₃ and aqueous NaCl is necessary when boronic acid B5 was used.

TABLE 1
Selected results of reaction conditions optimization for the synthesis of C3-O-acylate
using aldehydes as acylation reagents.

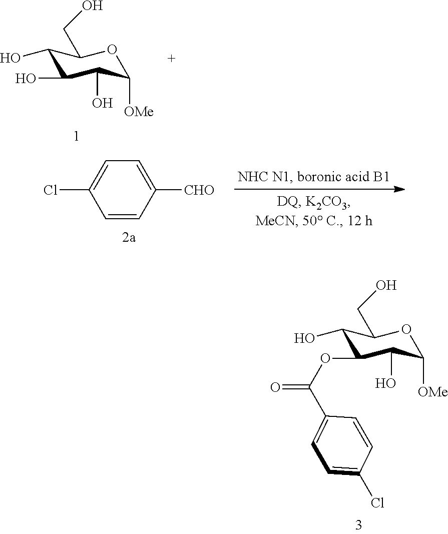

| | | ratio | yield^{c} |
|---|---|---|---|
| entry | variation from standard conditions | (C2:C3:C4:C6) | (%) |
| 1 | none^{a} | 1:5.8:0.5:0.5 | 78 |
| 2 | B1 (0 equiv) instead of 1.0 equiv | 1:2.3:0.6:2.3 | 38 |
| 3 | B1 (0.1 equiv) instead of 1.0 equiv | 1:3:0.8:1.6 | 45 |
| 4 | B1 (0.3 equiv) instead of 1.0 equiv | 1:2.7:0.5:1.5 | 57 |
| 5 | B1 (0.5 equiv) instead of 1.0 equiv | 1:3.4:0.6:1.1 | 56 |
| 6 | B1 (1.5 equiv) instead of 1.0 equiv | 1:7:0:0 | 72 |
| 7 | B1 (2 equiv) instead of 1.0 equiv | 1:7:0:0 | 75 |
| 8 | B1 (3 equiv) instead of 1.0 equiv | 1:8:0:0 | 79 |
| 9 | Trifluoroacetic acid (TFA, 1 equiv) | — | 0 |
| | instead of B1 | | |
| 10 | HOAc (1 equiv) instead of B1 | 1:7.0:0:0 | 20 |
| 11 | rt | 1:10:0:0 | 47 |
| 12 | 70° C. | 1:8:0:0 | 63 |
| 13 | NEt₃ (0.2 equiv) instead of K₂CO₃ | 1:11:0:0 | 61 |
| 14 | 1,8-Diazabicyclo[5.4.0]undec-7-ene | 1:6:0:0 | 60 |
| | (DBU, 0.2 equiv) instead of K₂CO₃ | | |
| 15 | NaOAc (0.2 equiv) instead of K₂CO₃ | 1:10:0:0 | 68 |
| 16 | K₂CO₃ (1 equiv) instead of 0.2 equiv | 1:5:0:0 | 32 |
| 17 | MnO₂ (1 equiv) instead of DQ | 1:4:0:0 | 21 |
| 18 | Phenyliodonium diacetate (PIDA, 1 | 1:3:0:0 | 9 |
| | equiv) instead of DQ | | |
| 19 | 2-lodoxybenzoic acid (IBX, 1 equiv) | 1:9:0:0 | 21 |
| | instead of DQ | | |
| 20 | THF | 1:5:0:0 | 31 |
| 21 | DCM | 1:14:0:0 | 15 |
| 22 | Toluene | 1:20:0:0 | 10 |
| 23 | Dimethylformamide (DMF) | 1:4.6:0:0 | 50 |
| 24 | DMSO | 1:4:0:0 | 33 |
| 25 | Ethyl acetate (EtOAc) | 1:10:0:0 | 74 |
| 26 | Acetone | 1:10:0:0 | 58 |
| 27 | MeOH | — | 0 |
| 28 | 1,4-dioxane | 1:4:0:0 | 34 |
| 29 | B1 (1.5 equiv), DQ (1.5 equiv) | 1:14:0:0 | 62 |
| | instead of 1.0 equiv | | |
| 30^{b} | B1 (1.5 equiv), DQ(1.5 equiv) | 1:16:0:0 | 70 |
| | instead of 1.0 equiv and EtOAc | | |
| | instead of acetonitrile (MeCN) | | |

^{a}Reaction conditions: 1 (0.1 mmol), 2a (0.2 mmol, 2.0 equiv), N1 (10 mol %), B1 (1.0 equiv), DQ (1.0 equiv), K₂CO₃ (0.2 equiv), MeCN (2 mL), 50° C., 12 h.
^{b}Reaction conditions: 1 (0.1 mmol), 2a (0.2 mmol, 2.0 equiv), N1 (10 mol %), B1 (1.5 equiv), DQ (1.5 equiv), K₂CO₃ (0.2 equiv), EtOAc (2 mL), 50° C., 12 h.
^{c}NMR yield of total acylates.

Selective acylation with carboxylic acids as acylation reagents (General Procedure B)

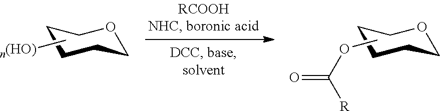

Monosaccharide (0.1 mmol, 1.0 equiv), carboxylic acid (0.2 mmol, 2.0 equiv), NHC catalyst (20 mol %), boronic acid (0.1 mmol, 1.0 equiv), dicyclohexyl carbodiimide (DCC, 0.2 mmol, 2.0 equiv), and base (0.2 mmol, 2.0 equiv) were added to a 4 mL screwtop test tube. Then, solvent (2 mL) was added to the mixture. The reaction mixture was allowed to stir vigorously at 50° C. for 12 h under a N₂ atmosphere. After cooling to room temperature, the reaction mixture was filtered, and then directly purified by silica gel flash column chromatography with an appropriate solvent (EtOAc/hexane 1:5 to 5:1 v/v) to afford the pure product.

TABLE 2
Selected results of reaction conditions optimization for the synthesis
of C3—O-acylate using carboxylic acids as acylation reagents.

| | variation from | ratio | yield^{c} |
|---|---|---|---|
| entry | standard conditions | (C2:C3:C4:C6) | (%) |
| 1 | none^{a} | 1:14:0:0 | 60 |
| 2 | without N1 | 1:0.4:0:0 | 14 |
| 3 | without B1 | 1:12:1:1 | 15 |
| 4 | without N1 and B1 | 1:1:0.3:0.3 | 8 |
| 5 | B3 instead of B1 | 1:1.5:0:0 | 55 |
| 6 | B6 instead of B1 | 0:100:0:0 | 4 |
| 7 | B7 instead of B1 | 1:3.7:0:0 | 14 |
| 8 | B8 instead of B1 | 1:1.5:0:0 | 53 |
| 9 | B10 instead of B1 | 1:3.3:0:0 | 57 |
| 10 | B11 instead of B1 | 1:5.5:1.5:0.5 | 17 |
| 11 | N4 instead of N1 | 1:4.4:0:0 | 27 |
| 12 | N5 instead of N1 | 1:2:0:0 | 40 |
| 13 | N6 instead of N1 | 1:1.5:0:0 | 25 |
| 14 | THF instead of EtOAc | 1:9:0:0 | 39 |
| 15 | acetone instead of EtOAc | 1:10:0.8:1 | 52 |
| 16 | DCM instead of EtOAc | 1:7.7:0.3:0.3 | 28 |
| 17 | MeCN instead of EtOAc | 1:8:0.5:0.3 | 63 |
| 18^{b} | NaOAc instead of Li₂CO₃ | 1:7:1:1 | 63 |
| 19^{b} | K₂CO₃ instead of Li₂CO₃ | — | trace |
| 20^{b} | Cs₂CO₃ instead of Li₂CO₃ | — | trace |
| 21^{b} | N,N-Diisopropylethylamine | — | trace |
| | (DIPEA) instead of Li₂CO₃ | | |
| 22^{b} | DBU instead of Li₂CO₃ | — | trace |
| 23^{b} | K₃PO₄ instead of Li₂CO₃ | — | trace |
| 24^{b} | DABCO instead of Li₂CO₃ | 1:3.5:0.4:0.5 | 60 |

^{a}Reaction conditions: 1 (0.1 mmol), carboxylic acid 2b (0.2 mmol, 2.0 equiv), NHC N1 (20 mol %), boronic acid B1 (1.0 equiv), DCC (2.0 equiv), Li₂CO₃ (2.0 equiv), EtOAc (2 mL), 50° C., 12 h.
^{b}MeCN as solvent.
^{c}NMR yield of total acylates.

Selective acylation with carboxylic esters as acylation reagents (General Procedure C)

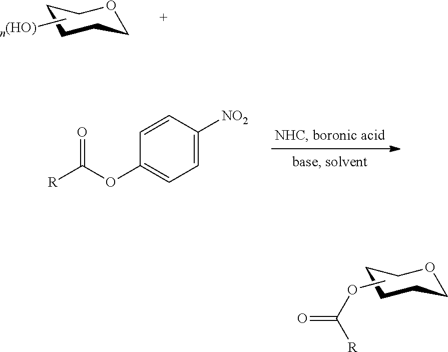

Monosaccharide (0.1 mmol, 1.0 equiv), ester (0.2 mmol, 2.0 equiv), NHC catalyst (10 mol %), boronic acid (1.0-1.5 equiv), and base (0.02 mmol, 0.2 equiv) were added to a 4 mL screwtop test tube. Then, solvent (2 mL) was added to the mixture. The reaction mixture was allowed to stir vigorously at 50° C. for 1-12 h under a N₂ atmosphere. Then, the reaction mixture was directly purified by flash column chromatography on silica with an appropriate solvent (EtOAc/hexane 1:5 to 5:1 v/v) to afford the pure product.

TABLE 3
Selected results of reaction conditions optimization for the synthesis of C3-O-acylate
using carboxylic esters as acylation reagents.

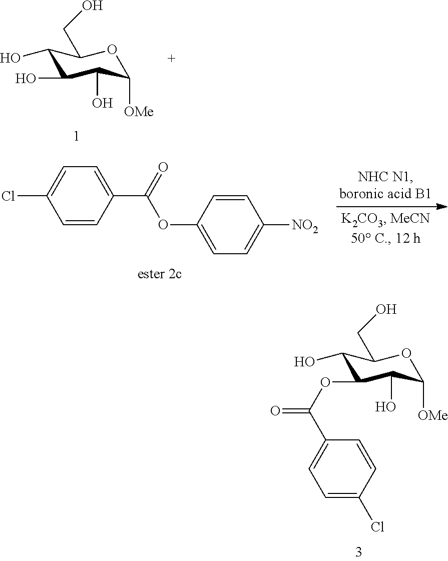

| | | ratio | yield^{c} |
|---|---|---|---|
| entry | variation from standard conditions | (C2:C3:C4:C6) | (%) |
| 1 | none^{a} | 1:4:0:0 | 70 |
| 2 | NHC N4 instead of NHC N1 | 1:1:0.3:0.4 | 45 |
| 3 | NHC N5 instead of NHC N1 | 1:1:0.1:0.1 | 43 |
| 4 | NHC N6 instead of NHC N1 | 1:0.9:0.1:0.2 | 36 |
| 5 | B5 instead of B1 | 1:3.5:0:0 | 18 |
| 6 | B6 instead of B1 | — | 0 |
| 7 | B7 instead of B1 | — | 0 |
| 8 | B8 instead of B1 | 1:0.7:0:0 | 49 |
| 9 | B10 instead of B1 | 1:1.2:0:0 | 55 |
| 10 | B11 instead of B1 | 1:2.1:0.7:1.1 | 49 |
| 11 | THF instead of MeCN | — | 0 |
| 12 | DCM instead of MeCN | 1:5.5:0:0 | 26 |
| 13 | acetone instead of MeCN | 1:3.8:0:0 | 48 |
| 14 | EtOAc instead of MeCN | 1:5:0:0 | 60 |
| 15^{b} | K₃PO₄ instead of K₂CO₃ | 1:5.7:0:0 | 67 |
| 16^{b} | NaOAc instead of K₂CO₃ | 1:5.8:0:0 | 61 |
| 17^{b} | Li₂CO₃ instead of K₂CO₃ | 0:23:0:0 | 23 |
| 18^{b} | DBU instead of K₂CO₃ | 1:5.4:0:0 | 64 |
| 19^{b} | DIPEA instead of K₂CO₃ | 1:6.2:0:0 | 72 |
| 20^{c} | without NHC N1 | — | 0 |
| 21^{c} | without B1 | 1:4:1:1 | 14 |
| 22^{c} | without NHC N1 and B1 | — | 0 |

^{a}Reaction conditions: 1 (0.1 mmol), ester 2c (0.2 mmol, 2.0 equiv), NHC N1 (10 mol %), boronic acid B1 (1.5 equiv), K₂CO₃ (0.2 equiv), MeCN (2 mL), 50° C., 12 h.
^{b}EtOAc as solvent.
^{c}DIPEA as base, EtOAc as solvent.
^{d}NMR yield of total acylates.

### Methyl-3-O-(4-chlorobenzoyl)-α-D-glucopyranoside (3)

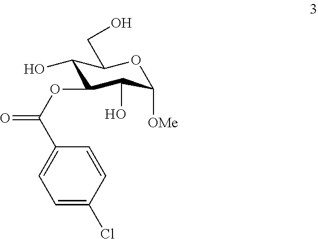

Following General Procedure A, the product 3 (26.5 mg, 80%) was obtained as a white solid. Following General Procedure B, the product 3 (23.6 mg, 71%) was obtained. Following General Procedure C, the product 3 (23.2 mg, 70%) was obtained.

¹H NMR (400 MHz, Chloroform-d) δ 8.04 (d, J=8.6 Hz, 2H), 7.46 (d, J=8.7 Hz, 2H), 5.34 (t, J=9.4 Hz, 1H), 4.88 (d, J=3.8 Hz, 1H), 3.99-3.88 (m, 2H), 3.84 (t, J=9.4 Hz, 1H), 3.78 (dq, J=10.1, 3.7 Hz, 2H), 3.52 (s, 3H). ¹³C NMR (101 MHz, Chloroform-d) δ 167.16, 140.01, 131.34, 128.83, 128.00, 99.45, 77.81, 71.45, 70.94, 69.26, 62.10, 55.56. ESI-MS: calcd for C₁₄H₁₇O₇CINa [M+Na]⁺: 355.0561, found: 355.0556.

### Methyl-2-O-(4-chlorobenzoyl)-α-D-alucopyranoside (39)

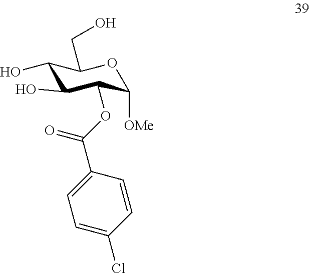

Following General Procedure A, the product 39 (20.9 mg, 63%) was obtained as a white solid. Following General Procedure C, the product 39 (20.0 mg) was obtained (total acylates 23.2 mg, 70%).

¹H NMR (400 MHz, Methanol-d₄) δ 8.13-7.98 (m, 2H), 7.58-7.39 (m, 2H), 5.01 (d, J=3.7 Hz, 1H), 4.86-4.82 (m, 1H), 3.99 (dd, J=10.0, 8.8 Hz, 1H), 3.89 (dd, J=11.9, 2.3 Hz, 1H), 3.75 (dd, J=11.9, 5.6 Hz, 1H), 3.64 (ddd, J=10.0, 5.6, 2.4 Hz, 1H), 3.51-3.45 (m, 1H), 3.41 (s, 3H). ¹³C NMR (101 MHz, Methanol-d₄) δ 165.34, 139.32, 131.03, 128.50, 128.44, 97.02, 74.36, 72.18, 71.10, 70.47, 61.15, 54.12. ESI-MS: calcd for C₁₄H₁₇O₇ CINa [M+Na]⁺: 355.0561, found: 355.0547.

### 6-O-(4-chlorobenzoyl)-D-galactal (57)

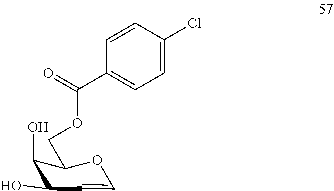

Following General Procedure A, the product 57 (17.0 mg, 60%) was obtained as a white solid. Following General Procedure C, the product 57 (15.9 mg, 56%) was obtained.

¹H NMR (400 MHz, Acetone-d₆) δ 8.11-7.94 (m, 2H), 7.65-7.45 (m, 2H), 6.35 (dd, J=6.3, 1.7 Hz, 1H), 4.73-4.62 (m, 2H), 4.56 (dd, J=11.6, 4.5 Hz, 1H), 4.42 (dt, J=4.4, 2.1 Hz, 1H), 4.33 (ddd, J=8.1, 4.5, 1.6 Hz, 1H), 4.03 (dt, J=4.7, 1.7 Hz, 1H). ¹³C NMR (101 MHz, Acetone-d₆) δ 164.93, 143.28, 138.88, 131.13, 128.97, 128.80, 103.23, 74.51, 65.31, 64.16, 63.10. ESI-MS: calcd for C₁₃H₁₃O₅CINa [M+Na]⁺: 307.0349, found: 307.0340.

### Methyl-3-O-(4-(N,N-diprooylsulfamoyl)benzoyl)-α-D-alucopyranoside (62)

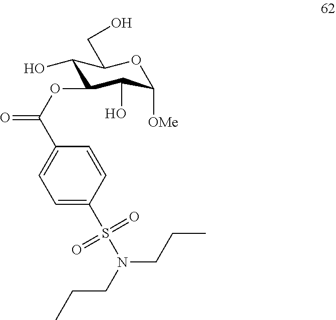

Following General Procedure B, the product 62 (33.2 mg, 72%) was obtained as a white solid. Following General Procedure C, the product 62 (28.6 mg) was obtained (total acrylates, 32.7 mg, 71%).

¹H NMR (500 MHz, Chloroform-d) δ 8.20 (d, J=8.6 Hz, 2H), 7.87 (d, J=8.5 Hz, 2H), 5.37 (t, J=9.5 Hz, 1H), 4.86 (d, J=3.8 Hz, 1H), 3.98-3.80 (m, 3H), 3.76 (tt, J=9.8, 3.8 Hz, 2H), 3.50 (s, 3H), 3.21-2.98 (m, 4H), 3.05 (s, 1H), 2.36 (d, J=11.2 Hz, 1H), 2.21 (s, 1H), 1.63-1.48 (m, 4H), 0.89 (t, J=7.4 Hz, 6H). ¹³C NMR (101 MHz, Chloroform-d) δ 166.39, 144.57, 132.95, 130.57, 127.00, 99.40, 78.00, 71.37, 70.93, 69.14, 62.07, 55.56, 49.92, 21.91, 11.15. ESI-MS: calcd for C20H32O9NS [M+H]⁺: 462.1798, found: 462.1799.

Results and discussion

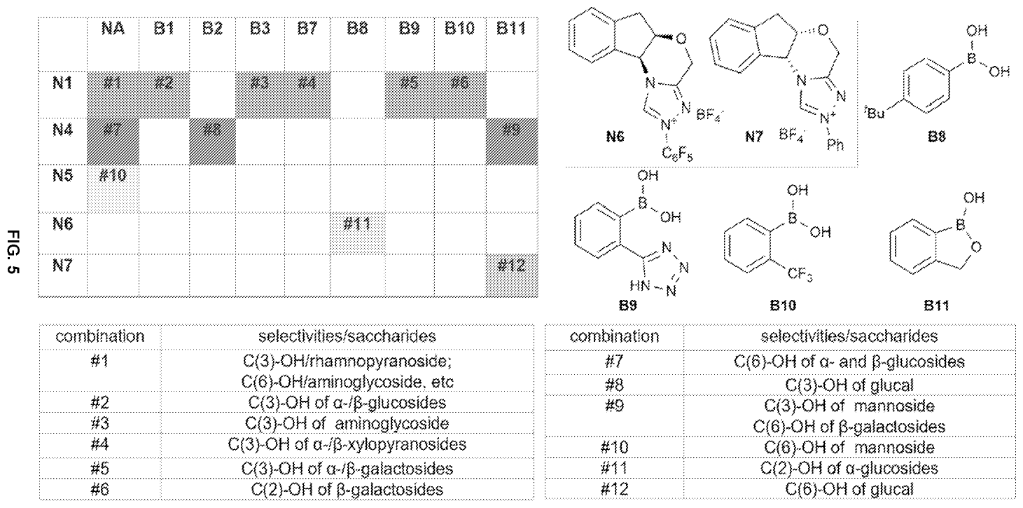

Summarized in FIG. 4 are key results of a model reaction (with D-glucoside (1) as the monosaccharide) from extensive studies on the effects of boronic acids, NHC catalysts, and other parameters such as bases and solvents.

### Example 3. Procedure for a gram scale reaction

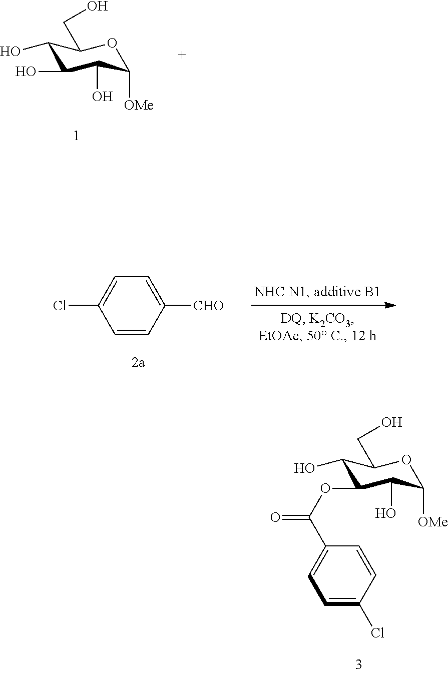

Monosaccharide 1 (5.0 mmol, 1.0 equiv), aldehyde 2a (10 mmol, 2.0 equiv), NHC N1 (10 mol %), boronic acid B1 (7.5 mmol, 1.5 equiv), DQ (7.5 mmol, 1.5 equiv), and K₂CO₃ (1.0 mmol, 0.2 equiv) was added to a 250 mL flask. Then, EtOAc (100 mL) was added to the mixture. The reaction mixture was allowed to stir vigorously at 50° C. for 12 h under a N₂ atmosphere. After cooling to room temperature, the reaction mixture was concentrated to 15 mL, and then directly purified by flash column chromatography on silica with an appropriate solvent (EtOAc/hexane 1:5 to 5:1 v/v) to afford 3 (1.16 g, 70%).

### Example 4. Thermodynamics for the formation of boronic ester from the condensation reaction between boronic acid and sugar

We computed the Gibbs energy of reaction for the condensation between boronic acid and monosaccharide.

Computation of Gibbs energy

For conformational sampling of structures, Grimme's crest program (Grimme, S., J. Chem. Theory Comput. 2019, 15, 2847-2862; and Pracht, P., Bohle, F. & Grimme, S., Phys. Chem. Chem. Phys. 2020, 22, 7169-7192), which used metadynamics (MTD) with genetic z-matrix crossing (GC) performed at the GFN2-xTB (Bannwarth, C., Ehlert, S. & Grimme, S., J. Chem. Theory Comput. 2019, 15,1652-1671; Grimme, S., Bannwarth, C. & Shushkov, P., J. Chem. Theory Comput. 2017, 13, 1989-2009; and Bannwarth, C. et al., WIREs Comput. Mol. Sci. 2021, 11, e1493) extended semiempirical tight-binding level of theory, was used. The resulting lowest energy structures were further optimized using global hybrid DFT functional M06-2X6 with Karlsruhe-family double-ζ valence def2-SVP (Weigend, F. & Ahlrichs, R., Phys. Chem. Chem. Phys. 2005, 7, 3297-3305; and Weigend, F. Phys. Chem. Chem. Phys. 2006, 8,1057-1065) basis set for all atoms as implemented in Gaussian 16 rev. B.01 (Frisch, M. J. et al., Gaussian 16, Revision B.01. 2016). Single point (SP) corrections were performed using M06-2X functional and def2-TZVP12 basis set for all atoms. Minima and transition structures on the potential energy surface (PES) were confirmed as such by harmonic frequency analysis, showing respectively zero and one imaginary frequency. The implicit SMD continuum solvation model (Marenich, A. V., Cramer, C. J. & Truhlar, D. G., J. Phys. Chem. B 2009, 113, 6378-6396) for acetonitrile solvent was used to account for the effect of solvent on the potential energy surface. Gibbs energies were evaluated at 50° C., which was used in the experiments, using a quasi-RRHO treatment of vibrational entropies (Luchini, G. et al., F1000Research 2020, 9, 291). Vibrational entropies of frequencies below 100 cm-1 were obtained according to a free rotor description, using a smooth damping function to interpolate between the two limiting descriptions (Grimme, S., Chem. Eur. J. 2012, 18, 9955-9964). The free energies were further corrected using standard concentration of 1 mol/L for gas-phase-to-solvent correction.

Results and discussion

The results are shown in Table 4. A general feature of our type of reaction, from the three reactions considered (where different monosaccharides, glucoside and galactoside, were used), is that the formation of boronic ester between the boronic acid and 4,6-diol of the sugar is exergonic (thermodynamically downhill), while that with 3,4-diol or 2,3-diol of the sugar are endergonic (thermodynamically uphill). This suggests that the formation with 4,6-diol of the sugar is favorable whereas the formations with 3,4-diol or 2,3-diol of the sugar are unfavorable. This means that under our reaction conditions where boronic acids can form boronic esters with monosaccharides, the hydroxyl groups at C4 and C6 will be involved in boronic ester formation, leaving hydroxyl groups at C2 and C3 exposed for subsequent acylation. We note that the hydroxyl groups on C4 and C6 can be of either cis-(as in galactoside) or trans-relationship (as in glucoside), without affecting this observation, as the C6 methylene group is flexible enough to ensure the formation of [6,6]-bicyclic rings in both cases. In addition, this observation is valid for all 3 boronic acids tested (B1, B9, B10, Table 4) and is likely to be valid for other boronic acids as well. The formation of [6,6]-bicyclic boronic ester is more stable than that of [5,6]-bicyclic boronic ester. From Table 4, we can see that for the reaction involving galactoside and boronic acid B9, the formation of boronic ester galactoside_B9_46diol is 2.9 kcal mol⁻¹ and 8.8 kcal mol⁻¹ more stable than boronic esters galactoside_B9_34diol and galactoside_B9_23diol, respectively. Similarly, for the reaction between galactoside and boronic acid B10, the formation of boronic ester galactoside_B10_46diol is 6.0 kcal mol⁻¹ and 13.1 kcal mol⁻¹ more stable than boronic esters galactoside_B10_34diol and galactoside_B10_23diol, respectively. For the reaction between glucoside and boronic ester B1, the formation of boronic ester glucoside_B1_46diol is 9.6 kcal mol⁻¹ and 9.7 kcal mol⁻¹ more stable than boronic esters glucoside_B1_34diol and glucoside_B1_23diol, respectively. The boronic ester formed with 4,6-diol of the sugar is expected to be the dominant species present and subsequently takes part in the reaction. This is consistent with the experimental verification of the involvement of boronic ester formed with 4,6-diol of the sugar (intermediates I and III) in the reaction between glucoside 1 with NHC N1 and boronic acid B1 as shown in Example 6 below. We conclude that for our reaction protocols, where boronic acids employed can form boronic ester with the monosaccharide, the most stable adduct that reacts further in the reaction will be the boronic acid-4,6-diol adduct, leaving only exposed OH groups at C2 and C3 for selective acylation.

TABLE 4
Computed Gibbs energy of reaction for the condensation between monosaccharides
and the boronic acids.

| | | | ΔGᵣ/kcal |
|---|---|---|---|
| Sugar | boronic acid | boronic ester, X | mol⁻¹ |
| 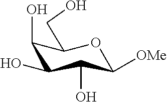 | 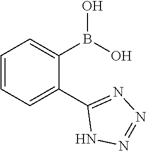 [SMILES: OB(O)c1ccccc1-c1nnn[nH]1] | 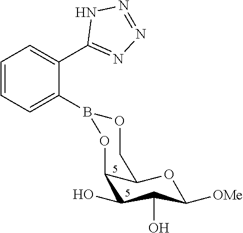 | −1.0 |
| galactoside | B9 | galactoside_B9_46diol | |
| | | 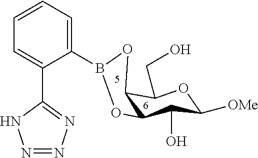 | 1.9 |
| | | galactoside_B9_34diol | |
| | | 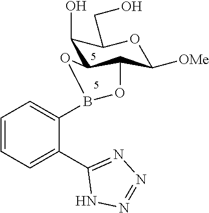 | 7.8 |
| | | galactoside_B9_23diol | |
| 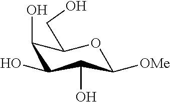 | 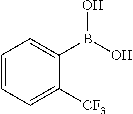 [SMILES: OB(O)c1ccccc1C(F)(F)F] | 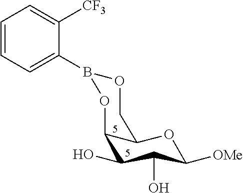 | −5.0 |
| galactoside | B10 | galactoside_B10_46diol | |
| | | 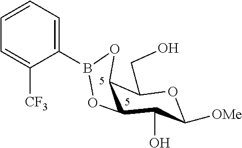 | 1.0 |
| | | galactoside_B10_34diol | |
| | | 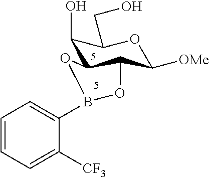 | 8.1 |
| | | galactoside_B10_23diol | |
| 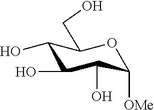 | 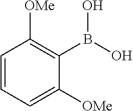 [SMILES: COc1cccc(OC)c1B(O)O] | 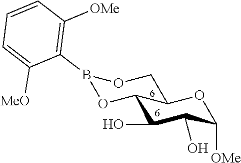 | −5.1 |
| glucoside | B1 | glucoside_B1_46diol | |
| | | 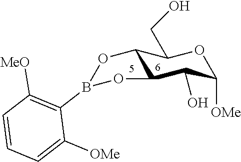 | 4.5 |
| | | glucoside_B1_34diol | |
| | | 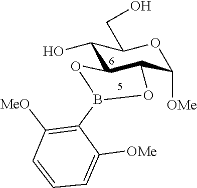 | 4.6 |
| | | glucoside_B1_23diol | |

### Example 5. Reaction mechanistic pathway of C2-OH selective acylation

To gain insight into the reaction mechanism, preliminary mechanistic studies on 02-OH selective acylation were conducted.

Identification of the intermediates in C2-OH selective acylation

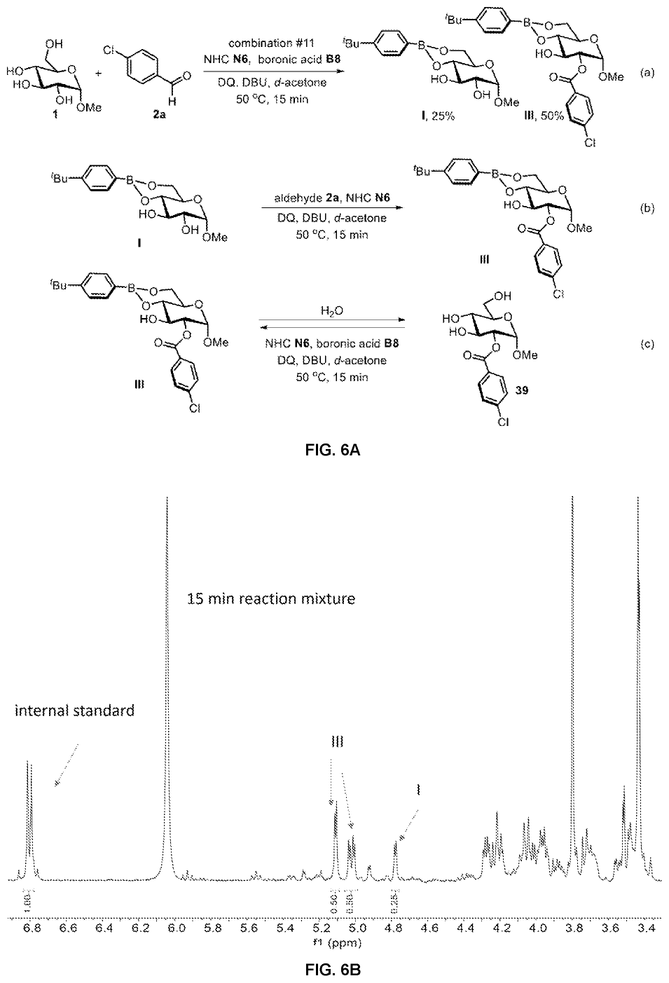

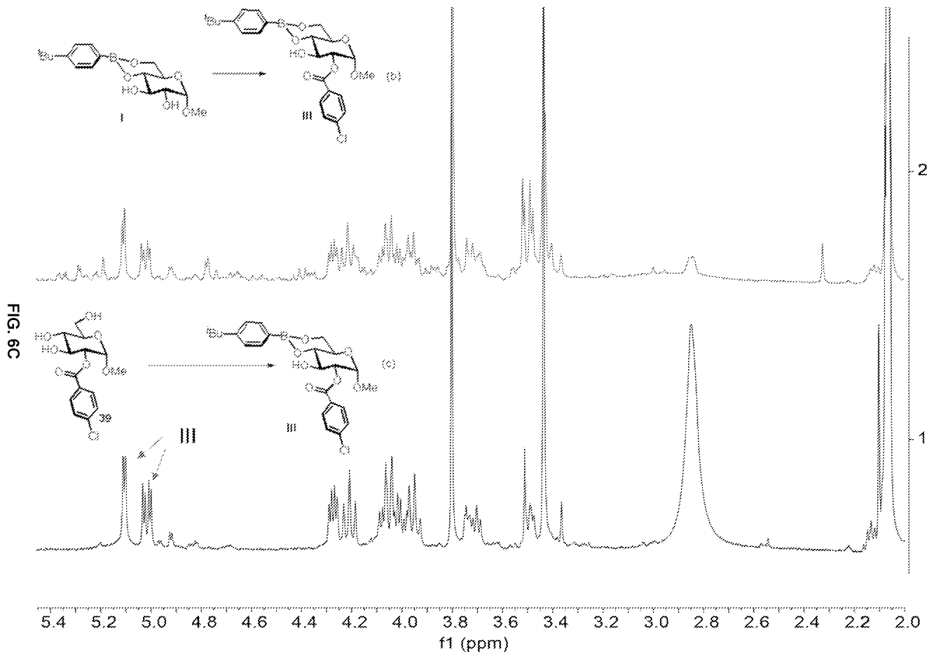

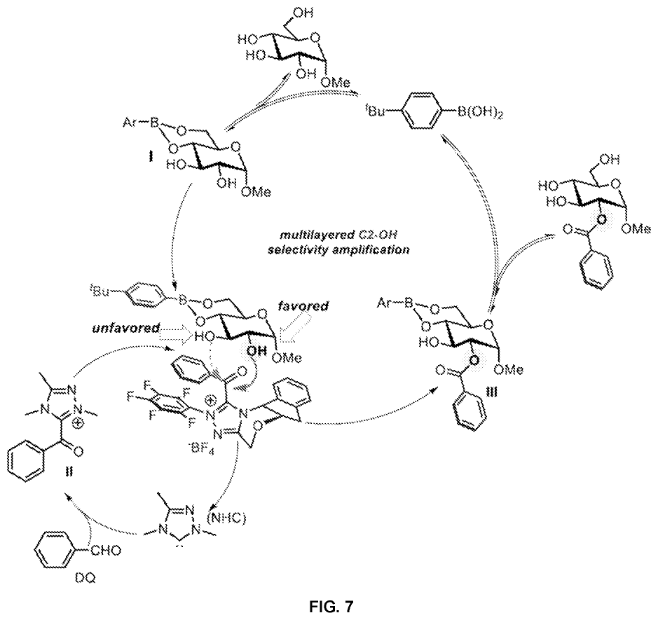

The reaction employing glucoside 1 and aldehyde 2a as substrates was conducted in d-acetone for 15 min by using N6 and B8 (combination #11, FIG. 5). Intermediate I and adduct Ill were efficiently afforded respectively in 25% and 50% NMR yield, clearly demonstrating that boronic esters were generated in the reaction (FIG. 6A, eq a). Intermediate I (Rocheleau, S. et al., Eur. J. Org. Chem. 2017, 2017, 646-656) previously prepared by heating boronic acid B8 with glucoside 1 in toluene with removal of water proposed in the 0(2)-OH selective acylation with NHC N6 as pre-catalyst to gain adduct Ill (FIG. 6A, eq b), which is unstable and could be instantaneously converted into the final product 39 in the presence of water (FIG. 6A, eq c). 39 as the substrate could also give adduct III under otherwise identical condition (FIG. 6A, eq c), suggesting that this process is reversible in the reaction.

### Preparation of the intermediate I in C2-OH selective acylation (Rocheleau, S. et al., Eur. J. Org. Chem. 2017, 2017, 646-656)

A suspension of glucoside 1 (1 mmol) in toluene (10 mL) was heated at reflux for 1 h using a Dean-Stark apparatus. Then, arylboronic acid B8 (1.2 equiv) was added, and the reaction mixture was heated at reflux for 1 h using a Dean-Stark apparatus. Then, the solution was cooled to room temperature and the solvent was evaporated under vacuum. The crude material was dissolved in CH₂C₁₂, and the solution was filtered and concentrated under vacuum. The solid residue was dissolved in a minimum amount of boiling toluene. The resulting solution was cooled to room temperature to give methyl 4,6-boronato-α-D-glucopyranoside, I.

¹H NMR (400 MHz, Acetone-d₆) δ 7.74 (d, J=8.3 Hz, 2H), 7.40 (d, J=8.4 Hz, 2H), 4.78 (d, J=3.7 Hz, 1H), 4.54 (d, J=3.7 Hz, 1H), 4.20 (dd, J=9.7, 4.9 Hz, 1H), 3.96 (t, J=10.1 Hz, 1H), 3.92-3.76 (m, 3H), 3.72 (t, J=9.2 Hz, 1H), 3.59-3.50 (m, 1H), 3.43 (s, 3H), 1.32 (s, 9H).

### Preparation of the adduct III in C2-OH selective acylation (Rocheleau, S. et al., Eur. J. Org. Chem. 2017, 2017, 646-656)

A suspension of 39 (1 mmol) in toluene (10 mL) was added arylboronic acid B8 (1.2 equiv). The reaction mixture was heated at reflux for 3 h using a Dean-Stark apparatus. Then, the solution was cooled to room temperature and evaporated under vacuum to give III as a crude material.

¹H NMR (400 MHz, Acetone-d₆) δ 8.10 (d, J=8.6 Hz, 2H), 7.75 (d, J=8.3 Hz, 2H), 7.61 (d, J=8.6 Hz, 2H), 7.47-7.33 (m, 2H), 5.11 (d, J=3.8 Hz, 1H), 5.02 (dd, J=9.7, 3.8 Hz, 1H), 4.31-4.25 (m, 1H), 4.21 (t, J=9.2 Hz, 1H), 4.10-3.91 (m, 3H), 3.44 (s, 3H), 1.33 (s, 9H).

Identification of the intermediate I and adduct III in C2-OH selective acylation (FIG. 6A, eq a): Glucoside 1 (0.1 mmol, 1.0 equiv), aldehyde 2a (0.2 mmol, 2.0 equiv), NHC catalyst N6 (10 mol %), boronic acid B8 (1.5 equiv), DQ (1.5 equiv), and DBU (0.02 mmol, 0.2 equiv) were added to a 4 mL screwtop test tube. Then, d-acetone (2 mL) was added to the mixture. The reaction mixture was allowed to stir vigorously at 50° C. for 15 min under a N₂ atmosphere. After the reaction mixture was cooled to room temperature, paraiodoanisole (0.05 mmol) as the internal standard was added to measure the NMR yield of the intermediate.

Possible transformation in the C2-OH selective acylation reaction (FIG. 6A, eq b)

Intermediate 1 (0.1 mmol, 1.0 equiv), aldehyde 2a (0.2 mmol, 2.0 equiv), NHC catalyst N6 (10 mol %), DQ (1.5 equiv), and DBU (0.02 mmol, 0.2 equiv) were added to a 4 mL screwtop test tube. Then, d-acetone (2 mL) was added to the mixture. The reaction mixture was allowed to stir vigorously at 50° C. for 15 min under a N₂ atmosphere. Then, the reaction mixture was cooled to room temperature to measure the ¹H NMR spectrum (FIG. 6A, eq b).

Possible transformation in the C2-OH selective acylation reaction (FIG. 6A, eq c)

39 (0.1 mmol, 1.0 equiv), NHC catalyst N6 (10 mol %), boronic acid B8 (1.5 equiv), DQ (1.5 equiv), and DBU (0.02 mmol, 0.2 equiv) were added to a 4 mL screwtop test tube. Then, d-acetone (2 mL) was added to the mixture. The reaction mixture was allowed to stir vigorously at 50° C. for 15 min under a N₂ atmosphere. Then, the reaction mixture was cooled to room temperature to measure the ¹H NMR spectrum (FIG. 6A, eq c).

Results and discussion

Based on the literature precedent and these experiments on the identification of the intermediates in C2-OH selective acylation, a plausible catalytic cycle is proposed in FIG. 7. Boronic ester I, generating via reversible reactions between C4 and C6 hydroxyl groups of glucoside with boronic acid, could react with the intermediate II generated from NHC catalyst and aldehyde 2a under the oxidation of DQ to give the adduct III and regenerate the NHC catalyst. The proper match of monosaccharide, NHC and boronic acid provides providential stereo electronic effects and colvalent/noncovalent interactions to site-specifically amplify C2-OH and attenuate other OHs selective acylation. Eventually, the adduct III undergoes hydrolysis in the same reaction mixture or in the presence of water to eventually form the site-selective acylated saccharide product and regenerate boronic acid.

### Example 6. Reaction mechanistic pathway of C3-OH selective acylation

To gain insight into the reaction mechanism, preliminary mechanistic studies on C3-OH selective acylation were also conducted.

Identification of the intermediates in C3-OH selective acylation

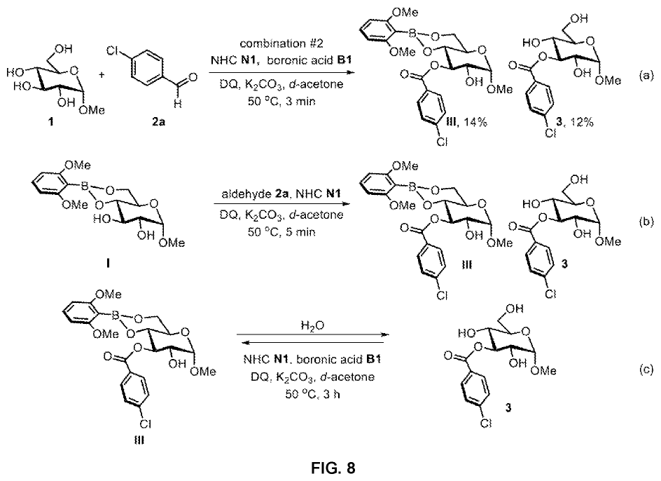

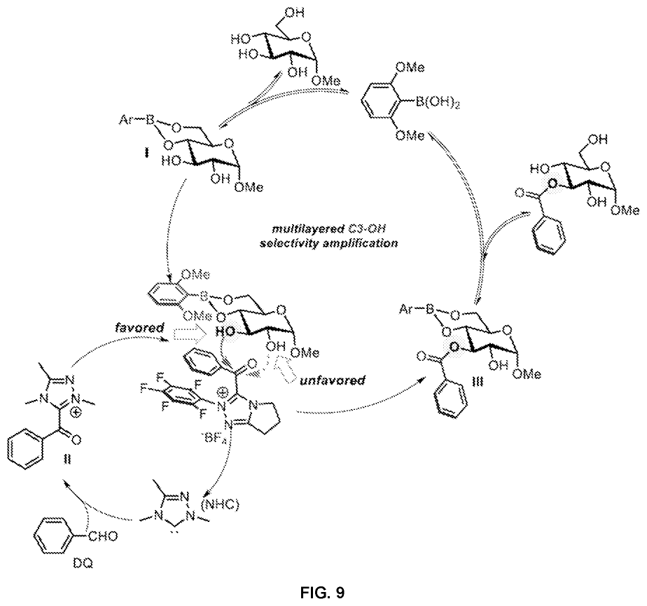

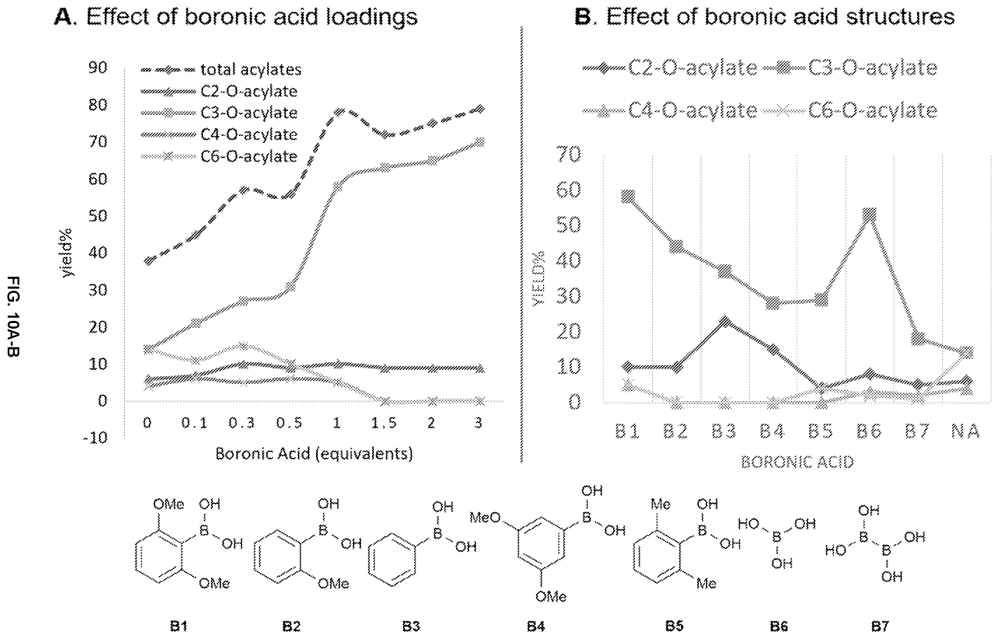

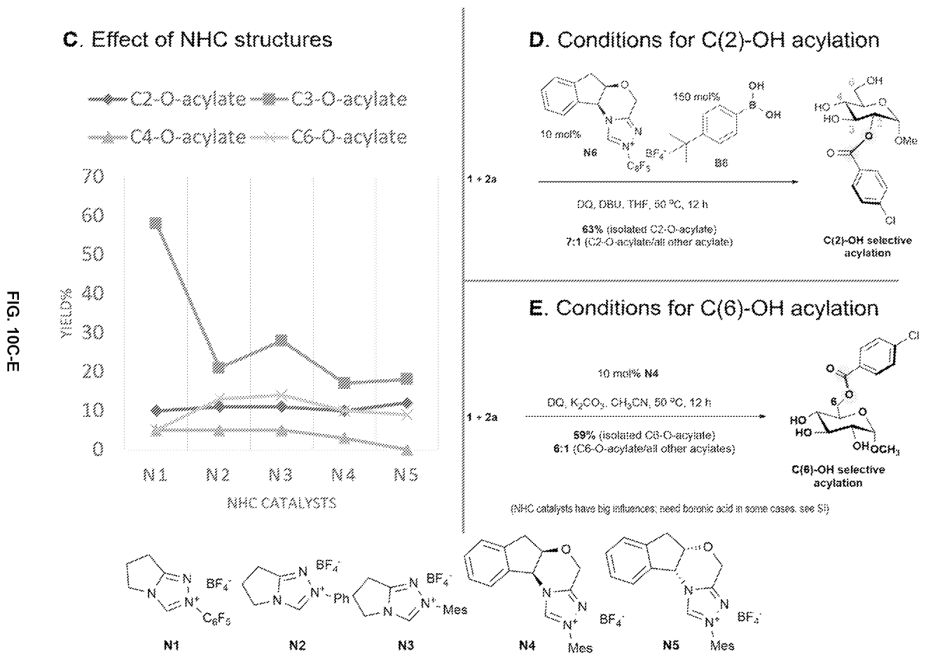

The reaction employing glucoside 1 and aldehyde 2a as substrates was conducted in d-acetone for 3 minutes by using N1 and B1 (combination #2, FIG. 5). Adduct III and product 3 were efficiently afforded respectively in 14% and 12% NMR yield, clearly demonstrating that boronic ester was generated in the reaction (FIG. 8, eq a). Intermediate I (FIG. 4) previously prepared by heating boronic acid B1 with glucoside 1 in toluene with removal of water proposed in the C(3)-OH selective acylation with NHC N1 as the pre-catalyst to gain adduct III (FIG. 8, eq b), which is unstable and could be instantaneously converted into the final product 3 in the same reaction mixture or in the presence of water (FIG. 8, eq b, c). 3 as the substrate could also give adduct III under otherwise identical condition (FIG. 8, eq c), suggesting that this process is reversible in the reaction.

### Preparation of the intermediate I in C3-OH selective acylation (Rocheleau, S. et al., Eur. J. Org. Chem. 2017, 2017, 646-656)

A suspension of glucoside 1 (1 mmol) in toluene (10 mL) was heated at reflux for 1 h using a Dean-Stark apparatus. Then, arylboronic acid B1 (1.2 equiv) was added, and the reaction mixture was heated at reflux for 1 h using a Dean-Stark apparatus. Then, the solution was cooled to room temperature and the solvent was evaporated under vacuum to give I as a crude material, which is very unstable and undergoes hydrolysis quickly in the NMR test tube.

¹H NMR (400 MHz, Acetone-d₆) δ 4.77 (d, J=3.8 Hz, 1H) (C1-H). As this intermediate is unstable, only the characteristic NMR signal is provided here.

### Preparation of the adduct III in C3-OH selective acylation (Rocheleau, S. et al., Eur. J. Org. Chem. 2017, 2017, 646-656)

A suspension of 3 (1 mmol) in toluene (10 mL) was added arylboronic acid B1 (1.2 equiv). The reaction mixture was heated at reflux for 3 h using a Dean-Stark apparatus. The solution was cooled to room temperature and evaporated under vacuum to give III as a crude material, which is very unstable and undergoes hydrolysis quickly in the NMR test tube.

¹H NMR (400 MHz, Acetone-d₆) δ 5.52 (t, J=9.4 Hz, 1H) (C3-H), 4.91 (d, J=3.6 Hz, 1H) (C1-H). As this intermediate is unstable, only the characteristic NMR signals are provided here.

Identification of the adduct III in C3-OH selective acylation (FIG. 8, eq a)

Glucoside 1 (0.1 mmol, 1.0 equiv), aldehyde 2a (0.2 mmol, 2.0 equiv), NHC catalyst N1 (10 mol %), boronic acid B1 (1 equiv), DQ (1 equiv), and K₂CO₃ (0.02 mmol, 0.2 equiv) were added to a 4 mL screwtop test tube. Then, d-acetone (2 mL) was added to the mixture. The reaction mixture was allowed to stir vigorously at 50° C. for 3 minutes under a N₂ atmosphere. After the reaction mixture was cooled to room temperature, paraiodoanisole (0.05 mmol) as the internal standard was added to measure the NMR yield of the intermediate.

Possible transformation in the C3-OH selective acylation reaction (FIG. 8, eq b)

Intermediate 1 (0.1 mmol, 1.0 equiv), aldehyde 2a (0.2 mmol, 2.0 equiv), NHC catalyst N1 (10 mol %), DQ (1 equiv), and K₂CO₃ (0.02 mmol, 0.2 equiv) were added to a 4 mL screwtop test tube. Then, d-acetone (2 mL) was added to the mixture. The reaction mixture was allowed to stir vigorously at 50° C. for 5 min under a N₂ atmosphere. Then, the reaction mixture was cooled to room temperature to measure the ¹H NMR spectrum (FIG. 8, eq b).

Possible transformation in the C3-OH selective acylation reaction (FIG. 8, eq c)

3 (0.1 mmol, 1.0 equiv), NHC catalyst N1 (10 mol %), boronic acid B1 (1 equiv), DQ (1 equiv), and K₂CO₃ (0.02 mmol, 0.2 equiv) were added to a 4 mL screwtop test tube. Then, d-acetone (2 mL) was added to the mixture. The reaction mixture was allowed to stir vigorously at 50° C. for 3 h under a N₂ atmosphere. Then, the reaction mixture was cooled to room temperature to measure the ¹H NMR spectrum (FIG. 8, eq c).

Results and discussion

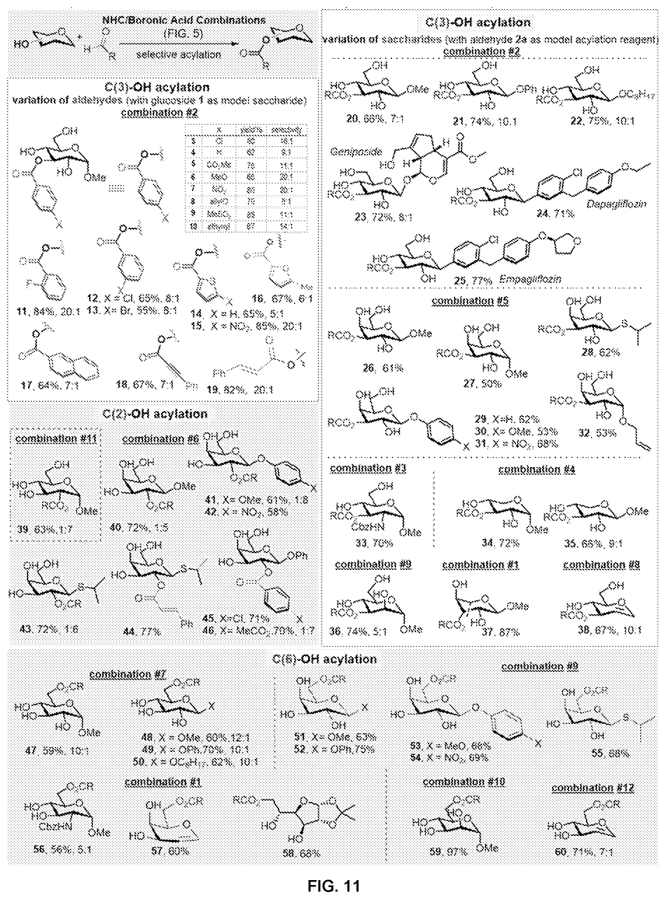

Based on these experiments on the identification of the intermediates in C3-OH selective acylation, a plausible catalytic cycle is proposed in FIG. 9. Boronic ester I, generating via reversible reactions between C4 and C6 hydroxyl groups of glucoside with boronic acid, could react with the intermediate II generated from the NHC catalyst and the aldehyde 2a under the oxidation of DQ to give the adduct III and regenerate the NHC catalyst. The proper match of monosaccharide, NHC and boronic acid provides providential stereo electronic effects and covalent/NCIs to site-specifically amplify C3-OH and attenuate other OHs selective acylation. Eventually, the adduct III undergoes hydrolysis in the same reaction mixture or in the presence of water to eventually form the site-selective acylated saccharide product and regenerate boronic acid.

The C3-OH selective acylation reaction and its simplified mechanistic pathway are briefly illustrated in FIG. 4. A glucoside (1), acylation substrate (2a, 2b or 2c), NHC pre-catalyst (N1, 10-20 mol %), boronic acid (B1, 100 mol %), and base (20-200 mol %) were dissolved in an organic solvent (e.g., CH₃CN or EtOAc). The reaction involves reversible reactions between two OH groups (C4- and C6-OH groups) of glucoside with boronic acid to form boronic ester I as an intermediate (detectable via ¹H NMR of the crude reaction mixture or isolable depending on the specific substrates).

The dynamic boronic ester formation not only provides a transient protection of the two OH groups from subsequent acylation reactions but also assists in regulating the acylation tendency of other OH groups by varying the substituents of boronic acids. In the same reaction solution, the NHC catalyst reacts with the acylation substrate to form acyl azolium intermediate II. The acylation substrates in our studies (as precursors of acyl azolium intermediates) can be aldehydes (2a; in the presence of an oxidant, such as DQ), carboxylic acids (2b; in the presence of a coupling reagent such as DCC), or carboxylic esters (2c) (FIG. 4 and see Example 2). The acylation reaction between intermediates I and II first forms an acylated boronic ester of the glucoside as adduct III (as observed via ¹H NMR analysis of the crude reaction mixture). In this step (from intermediates I and II to adduct III), the regioselectivity between the C(2)-OH and C(3)-OH moieties is controlled by the structures of both the NHC catalyst and the boronic acid. The boronic ester moiety of this adduct (III) then undergoes hydrolysis in the same reaction mixture or upon silica-gel column chromatography to eventually form the site-selective acylated saccharide product 3. It is worth noting that the boronic ester formation (thermodynamically favorable under the reaction condition) and hydrolysis are a facile and reversible process for both intermediates I and III (see Examples 4-6 for details). It is technically necessary to use a stoichiometric amount of the boronic acid to achieve optimal regioselectivity and avoid over-acylation on more than one OH group. Four sets of conditions (1A, 1B, 2A, and 3A; FIG. 4) were identified to give acceptable results. We chose condition 1B to study the effects of NHC catalysts and boronic acids given that four possible mono-acylated saccharide adducts could be observable under this type of condition (CH₃CN as solvent; 1 equiv of DQ and boronic acid).

### Example 7. Effects of NHCs and boronic acids on reaction yields and selectivity

Experimental procedure for FIG. 10A

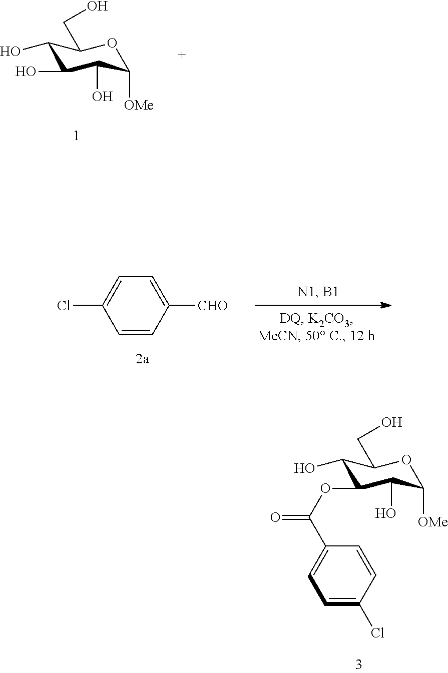

Monosaccharide 1 (0.1 mmol, 1.0 equiv), aldehyde 2a (0.2 mmol, 2.0 equiv), NHC N1 (10 mol %), boronic acid B1 (0-3.0 equiv), DQ (1.0 equiv), and K₂CO₃ (0.02 mmol, 0.2 equiv) were added to a 4 mL screwtop test tube. Then, MeCN (2 mL) was added to the mixture. The reaction mixture was allowed to stir vigorously at 50° C. for 12 h under a N₂ atmosphere. After cooling to room temperature, the reaction mixture was directly purified by flash column chromatography on silica with an appropriate solvent (EtOAc/hexane 1:5 to 1:0 v/v) to afford the mixture product. Paraiodoanisole (0.05 mmol) was used as the internal standard to measure the NMR yield (C2:C3:C4:C6).

Experimental procedure for FIG. 10B

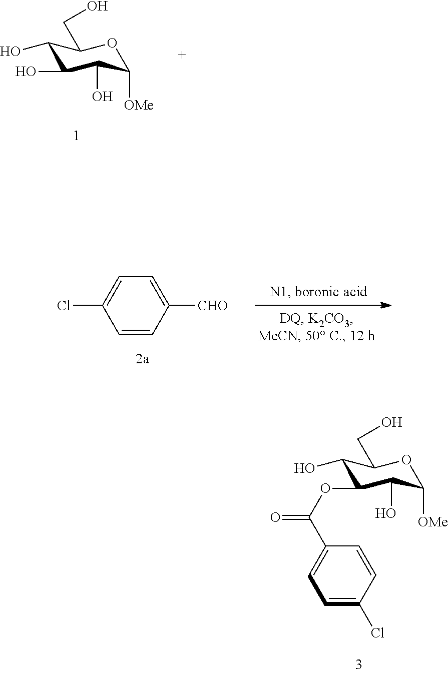

Monosaccharide 1 (0.1 mmol, 1.0 equiv), aldehyde 2a (0.2 mmol, 2.0 equiv), NHC N1 (10 mol %), boronic acid (1.0 equiv), DQ (1.0 equiv), and K₂CO₃ (0.02 mmol, 0.2 equiv) were added to a 4 mL screwtop test tube. Then, MeCN (2 mL) was added to the mixture. The reaction mixture was allowed to stir vigorously at 50° C. for 12 h under a N₂ atmosphere. After cooling to room temperature, the reaction mixture was directly purified by flash column chromatography on silica with an appropriate solvent (EtOAc/hexane 1:5 to 1:0 v/v) to afford the mixture product. Paraiodoanisole (0.05 mmol) was used as the internal standard to measure the NMR yield (C2:C3:C4:C6).

Experimental procedure for FIG. 10C

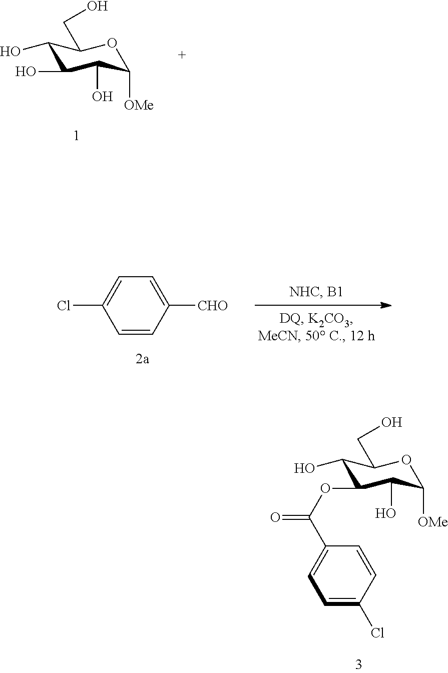

Monosaccharide 1 (0.1 mmol, 1.0 equiv), aldehyde 2a (0.2 mmol, 2.0 equiv), NHC catalyst (10 mol %), boronic acid B1 (1.0 equiv), DQ (1.0 equiv), and K₂CO₃ (0.02 mmol, 0.2 equiv) were added to a 4 mL screwtop test tube. Then, MeCN (2 mL) was added to the mixture. The reaction mixture was allowed to stir vigorously at 50° C. for 12 h under a N₂ atmosphere. After cooling to room temperature, the reaction mixture was directly purified by flash column chromatography on silica with an appropriate solvent (EtOAc/hexane 1:5 to 1:0 v/v) to afford the mixture product. Paraiodoanisole (0.05 mmol) was used as the internal standard to measure the NMR yield (C2:C3:C4:C6).

Results and discussion

The loadings of boronic acid (B1) had a clear influence on the reaction yields and selectivity (FIG. 10A). Increasing the loadings of boronic acid significantly increased the yield of C3-O-acylate but decreased those of C6-O-acylate and C4-O-acylate. The yield of C2-O-acylate remained largely unchanged as the boronic acid loadings were varied.

TABLE 5A
Experimental results for FIG. 10A.

| | | | | |
|---|---|---|---|---|
| 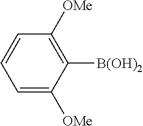 [SMILES: COc1cccc(OC)c1B(O)O] | 0 38% 6:14:4:14 | 0.1 45% 7:21:6:11 | 0.3 57% 10:27:5:15 | 0.5 56% 9:31:6:10 |
| dosage | | | | |
| (equiv) | | | | |

TABLE 5B
Experimental results for FIG. 10A.

| | | | | |
|---|---|---|---|---|
| 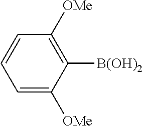 [SMILES: COc1cccc(OC)c1B(O)O] | 1.0 78% 10:58:5:5 | 1.5 72% 9:63:0:0 | 2 75% 9:65:0:0 | 3 79% 9:70:0:0 |
| dosage | | | | |
| (equiv) | | | | |

The structures of the boronic acids (as exemplified by selected examples B1-B7) also dramatically affected the yields and selectivity of the reactions (FIG. 10B). For example, removing the methoxy substituents on the phenyl ring of B1 (to give boronic acid B3) led to a big drop in the yields of C3-O-acylate and the ratios (selectivity) between C3-O-acylate and C2-O-acylate. The presence of boronic acid generally increased the overall acylation yields (e.g. 68% overall acylation yield with the presence of 1 equiv of B1 versus 38% overall yield without B1; FIG. 10A-B). These results suggest that the formation of boronic ester intermediate (I; FIG. 4) also simultaneously activates C(3)-OH toward acylation reactions. Such activation effects can be designed to occur on other OH groups, such as the C(2)-OH moiety, as observed in other examples of this study.

TABLE 6A
Experimental results for FIG. 10B.

| | 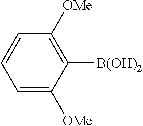 [SMILES: COc1cccc(OC)c1B(O)O] | 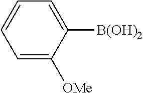 [SMILES: COc1ccccc1B(O)O] | 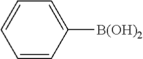 | 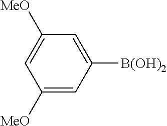 [SMILES: COc1cc(OC)cc(B(O)O)c1] |
|---|---|---|---|---|
| NHC\Boronic acid | B1 | B2 | B3 | B4 |
| 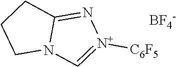 | 68% (10:58:5:5) | 54% (10:44:0:0) | 60% (23:37:0:0) | 43% (15:28:0:0) |
| N1 | | | | |

TABLE 6B
Experimental results for FIG. 10B.

| | 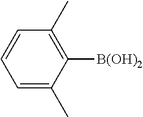 | | | |
|---|---|---|---|---|
| NHC\Boronic acid | B5 | B(OH)₃ B6 | B₂(OH)₄ B7 | NA |
| 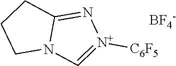 | 37% (4:29:0:4) | 66% (8:53:3:2) | 26% (5:18:2:1) | 38% (6:14:4:14) |
| N1 | | | | |

The structures of NHC catalysts also showed profound effects on both reaction yields and selectivity values (FIG. 100).

TABLE 7A
Experimental results for FIG. 10C.

| | 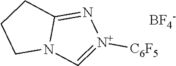 | 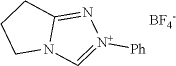 | 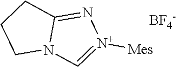 |
|---|---|---|---|
| NHC\Boronic acid | N1 | N2 | N3 |
| 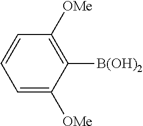 [SMILES: COc1cccc(OC)c1B(O)O] | 68% (10:58:5:5) | 50% (11:21:5:13) | 58% (11:28:5:14) |
| B1 | | | |

TABLE 7B
Experimental results for FIG. 10C.

| | 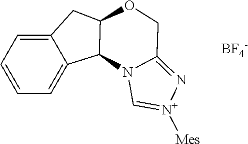 | 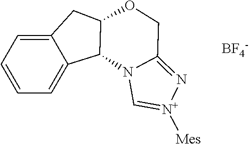 |
|---|---|---|
| NHC\Boronic acid | N4 | N5 |
| 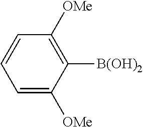 [SMILES: COc1cccc(OC)c1B(O)O] | 40% (10:17:3:10) | 39% (12:18:0:9) |
| B1 | | |

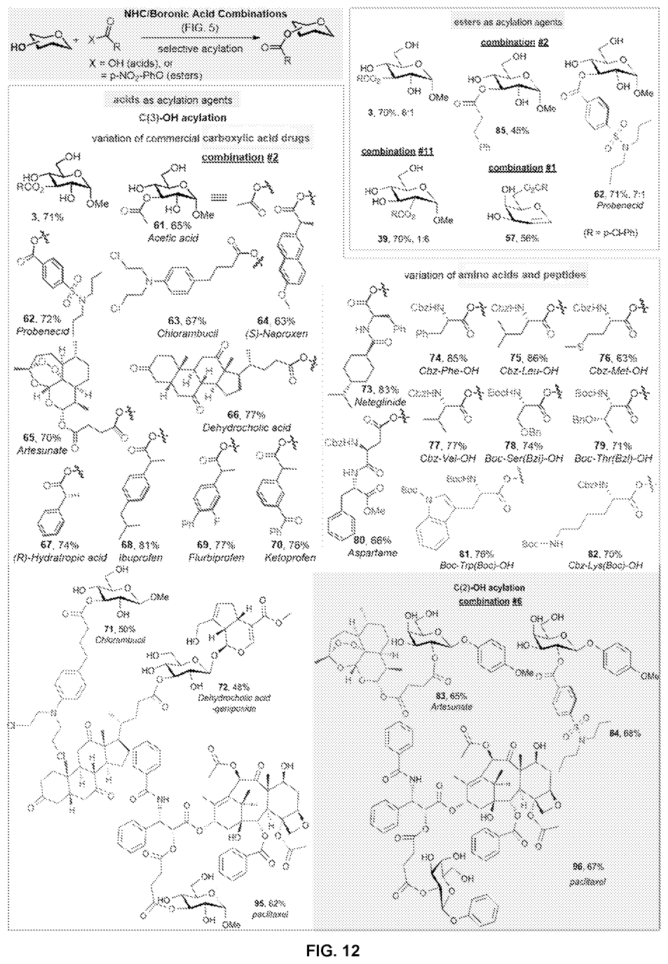

Our results (FIG. 10A-C) clearly show that both NHCs and boronic acids have distinct effects on each of the different OH groups present in saccharides and their analogs. These effects can be deactivation (temporary shielding) or activation on different OH groups, providing amplified reactivity differentiations of these moieties. It is therefore feasible to engineer these effects in a combinatorial and programmable manner (FIGS. 1B, 1I and 12) to achieve site-selective acylation on different OH groups of various types of saccharides (and polyols) with diverse acylation partners. For example, selective C(2)-OH acylation of glucoside (1) could be achieved by the combined use of chiral NHC pre-catalyst N6 and boronic acid B8 under a slightly modified condition to give C2(OH)-acylated product in 63% yield and 7:1 regioselectivity (FIGS. 5 and 10D). Acylation of C(6)-OH of glucoside (1) selectively was achieved with the NHC pre-catalyst (N4) alone (FIGS. 5 and 10E). In other examples of this study, selective C(6)-OH acylation was obtained via the combined use of an NHC pre-catalyst and a boronic acid.

### Example 8. Scope and applications of our strategy

We next evaluated the scope and applications of our strategy.

Conditions in using the different NHC/boronic acid combinations (FIG. 5) for the various acylation reactions

TABLE 8
Conditions in using the different NHC/boronic acid combinations (FIG. 5)
for the various acylation reactions to prepare products 3 to 84, 95, 96.

| | |
|---|---|
| NHC/boronic | products made |
| acid combination | under this |

| entry | (FIG. 5) | conditions | condition |
|---|---|---|---|
| 1 | #1 (N1) | aldehyde (0.2 mmol, 2.0 equiv), NHC | 37 |
| | | N1 (10 mol %), DQ (1.0 equiv), K₂CO₃ | |
| | | (0.2 equiv), MeCN (2 mL), room | |
| | | temperature (rt), 24 h | |
| | | ester 2c (0.2 mmol, 2.0 equiv), NHC N1 | 57 |
| | | (10 mol %), DBU (0.2 equiv), MeCN (2 | |
| | | mL), rt, 24 h | |
| | | aldehyde (0.2 mmol, 2.0 equiv), NHC | 56, 57, 58 |
| | | N1 (10 mol %), DQ (1.0 equiv), K₂CO₃ | |
| | | (0.2 equiv), MeCN (2 mL) | |
| 2 | #2 (N1, B1) | aldehyde (0.2 mmol, 2.0 equiv), NHC | 3-22, 24, 25 |
| | | N1 (10 mol %), boronic acid B1 (1.5 | |
| | | equiv), DQ (1.5 equiv), K₂CO₃ | |
| | | (0.2 equiv), EtOAc (2 mL) | |
| | | aldehyde (0.3 mmol, 3.0 equiv), NHC | 23 |
| | | N1 (10 mol %), boronic acid B1 (1.0 | |
| | | equiv), DQ (1.0 equiv), DBU (0.2 equiv), | |
| | | MeCN (2 mL) | |
| | | carboxylic ester (0.2 mmol, 2.0 equiv), | 3, 62, 85 |
| | | NHC N1 (10 mol %), boronic acid B1 | |
| | | (1.5 equiv), DIPEA (0.2 equiv), | |
| | | EtOAc (2 mL) | |

| | | |
|---|---|---|
| carboxylic acid | Li₂CO₃ (2.0 equiv), | 3, 61, 62 |
| (0.2 mmol, | EtOAc (2 mL) | |
| 2.0 equiv), | NaOAc (2.0 | 63-71, 73-82, |
| NHC N1 (20 | equiv), | 95 |
| mol %), boronic | MeCN (2 mL) | |
| acid B1 (1.0 | Li₂CO₃ (2.0 equiv), | 72 |
| equiv), DCC | MeCN (2 mL) | |
| (2.0 equiv) | | |

| | | | |
|---|---|---|---|
| 3 | #3 (N1, B3) | aldehyde (0.2 mmol, 2.0 equiv), NHC | 33 |
| | | N1 (10 mol %), boronic acid B3 (1.0 | |
| | | equiv), DQ (1.0 equiv), K₂CO₃ | |
| | | (0.2 equiv), MeCN (2 mL) | |
| 4 | #4 (N1, B7) | aldehyde (0.2 mmol, 2.0 equiv), NHC | 34, 35 |
| | | N1 (10 mol %), boronic acid B7 (1.0 | |
| | | equiv), DQ (1.0 equiv), K₂CO₃ | |
| | | (0.2 equiv), MeCN (2 mL) | |
| 5 | #5 (N1, B9) | aldehyde (0.2 mmol, 2.0 equiv), NHC | 26-32 |
| | | N1 (10 mol %), boronic acid B9 (1.0 | |
| | | equiv), DQ (1.0 equiv), DBU (0.2 equiv), | |
| | | THF (2 mL) | |
| 6 | #6 (N1, B10) | aldehyde (0.2 mmol, 2.0 equiv), NHC | 41-46 |
| | | N1 (10 mol %), boronic acid B10 (1.0 | |
| | | equiv), DQ (1.0 equiv), K₂CO₃ | |
| | | (0.2 equiv), MeCN (2 mL) | |
| | | aldehyde (0.2 mmol, 2.0 equiv), NHC | 40 |
| | | N1 (10 mol %), boronic acid B5 or | |
| | | B10(1.0 equiv), DQ (1.0 equiv), K₂CO₃ | |
| | | (0.2 equiv), acetone (2 mL) | |
| | | carboxylic acid (0.2 mmol, 2.0 equiv), | 83, 84, 96 |
| | | NHC N1 (20 mol %), boronic acid B10 | |
| | | (1.0 equiv), DCC (2.0 equiv), NaOAc | |
| | | (2.0 equiv), MeCN (2 mL) | |
| 7 | #7 (N4) | aldehyde (0.2 mmol, 2.0 equiv), NHC | 47-50 |
| | | N4 (10 mol %), DQ (1.0 equiv), K₂CO₃ | |
| | | (0.2 equiv), MeCN (2 mL) | |
| 8 | #8 (N4, B2) | aldehyde (0.2 mmol, 2.0 equiv), NHC | 38 |
| | | N4 (10 mol %), boronic acid B2 (1.0 | |
| | | equiv), DQ (1.0 equiv), DBU (0.2 equiv), | |
| | | THF (2 mL) | |
| 9 | #9 (N4, B11) | aldehyde (0.2 mmol, 2.0 equiv), NHC | 36 |
| | | N4 (10 mol %), boronic acid B11 (1.0 | |
| | | equiv), DQ (1.0 equiv), DBU (0.2 equiv), | |
| | | MeCN (2 mL) | |
| | | aldehyde (0.3 mmol, 3.0 equiv), NHC | 51-55 |
| | | N4 (20 mol %), boronic acid B11 (1.0 | |
| | | equiv), DQ (1.5 equiv), DBU (0.2 equiv), | |
| | | THF (2 mL) | |
| 10 | #10 (N5) | aldehyde (0.3 mmol, 3.0 equiv), NHC | 59 |
| | | N5 (20 mol %), DQ (1.5 equiv), DBU | |
| | | (0.2 equiv), THF (2 mL) | |
| 11 | #11 (N6, B8) | aldehyde (0.2 mmol, 2.0 equiv), NHC | 39 |
| | | N6 (10 mol %), boronic acid B8 (1.5 | |
| | | equiv), DQ (1.5 equiv), DBU (0.2 equiv), | |
| | | THF (2 mL) | |
| | | ester 2c (0.2 mmol, 2.0 equiv), NHC N6 | |
| | | (10 mol %), boronic acid B8 (1.5 equiv), | |
| | | DBU (0.2 equiv), THF (2 mL), rt, 24 h | |
| 12 | #12 (N7, B11) | aldehyde (0.2 mmol, 2.0 equiv), NHC | 60 |
| | | N7 (10 mol %), boronic acid B11 (1.0 | |
| | | equiv), DQ (1.0 equiv), DBU (0.2 equiv), | |
| | | THF (2 mL) | |

Common conditions (saccharide (0.1 mmol), 50° C., 12 h) are not given.

### Methyl-3-O-benzoyl-α-D-glucopyranoside (4)

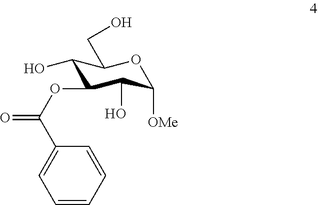

The product 4 (18.5 mg, 62%) was obtained as a white solid.

¹H NMR (500 MHz, Chloroform-d) δ 8.09 (dd, J=8.3, 1.4 Hz, 2H), 7.68-7.55 (m, 1H), 7.46 (t, J=7.7 Hz, 2H), 5.34 (t, J=9.4 Hz, 1H), 4.85 (d, J=3.8 Hz, 1H), 3.95-3.85 (m, 2H), 3.85-3.72 (m, 3H), 3.49 (s, 3H), 3.16 (s, 1H), 2.42 (d, J=10.5 Hz, 1H), 2.25 (s, 1H). ¹³C NMR (126 MHz, Chloroform-d) δ 168.10, 133.48, 129.96, 129.51, 128.45, 99.46, 77.59, 71.45, 70.95, 69.31, 62.10, 55.50. ESI-MS: calcd for C₁₄H₁₈O₇Na [M+Na]⁺: 321.0950, found: 321.0955.

### Methyl-3-O-(4-methoxycarbonyl benzoyl)-α-D-glucopyranoside (5)

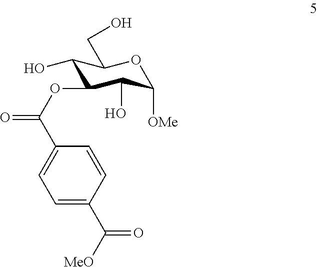

The product 5 (27.0 mg, 76%) was obtained as a white solid.

¹H NMR (500 MHz, Chloroform-d) δ 8.13 (q, J=8.5 Hz, 4H), 5.36 (t, J=9.5 Hz, 1H), 4.87 (d, J=3.8 Hz, 1H), 3.98 (s, 3H), 3.95-3.71 (m, 5H), 3.51 (s, 3H), 2.89 (d, J=4.8 Hz, 1H), 2.30 (d, J=11.0 Hz, 1H), 2.08 (s, 1H). ¹³C NMR (126 MHz, Chloroform-d) δ 167.08, 166.22, 134.31, 133.33, 129.90, 129.59, 99.42, 77.95, 71.41, 70.93, 69.25, 62.11, 55.56, 52.52. ESI-MS: calcd for C₁₆H₂₁O₉[M+H]⁺: 357.1186, found: 357.1190.

### Methyl-3-O-(4-methoxybenzoyl)-α-D-glucopyranoside (6)

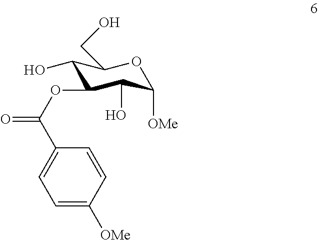

The product 6 (21.6 mg, 66%) was obtained as a white solid.

¹H NMR (400 MHz, Chloroform-d) δ 8.15-7.92 (m, 2H), 7.09-6.74 (m, 2H), 5.30 (t, J=9.1 Hz, 1H), 4.89 (d, J=3.8 Hz, 1H), 3.92 (s, 5H), 3.88-3.74 (m, 3H), 3.53 (s, 3H), 3.03 (s, 1H), 2.31 (d, J=10.7 Hz, 1H), 2.07 (s, 1H). ¹³C NMR (101 MHz, Chloroform-d) δ 168.10, 163.91, 132.13, 121.73, 113.77, 99.44, 77.62, 77.24, 71.50, 70.93, 69.66, 62.29, 55.52. ESI-MS: calcd for C₁₅H₂₁O₈[M+H]⁺: 329.1237, found: 329.1239.

### Methyl-3-O-(4-nitrobenzoyl)-α-D-glucopyranoside (7)

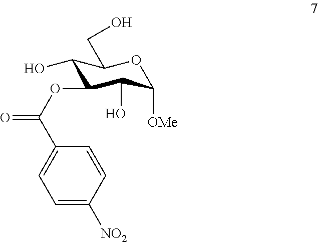

The product 7 (27.4 mg, 80%) was obtained as a white solid.

¹H NMR (400 MHz, Acetonitrile-d₃) δ 8.46-8.08 (m, 4H), 5.30 (dd, J=9.9, 8.8 Hz, 1H), 4.78 (d, J=3.7 Hz, 1H), 3.88-3.54 (m, 6H), 3.45 (s, 3H), 3.13 (d, J=8.5 Hz, 1H), 2.83 (s, 1H). ¹³C NMR (101 MHz, Acetonitrile-d₃) δ 164.70, 150.77, 135.85, 130.76, 123.67, 99.63, 77.81, 71.91, 70.33, 68.23, 61.23, 54.66. ESI-MS: calcd for C₁₄H₁₇O₉NNa [M+Na]⁺: 366.0801, found: 366.0782.

### Methyl-3-O-(4-(allyloxy)benzoyl)-α-D-alucopyranoside (8)

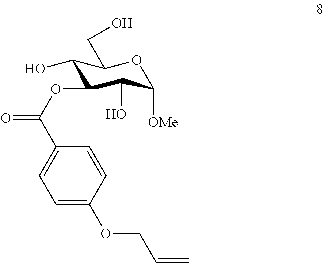

The product 8 (24.8 mg, 70%) was obtained as a white solid.

¹H NMR (400 MHz, Chloroform-d) δ 8.15-7.80 (m, 2H), 7.10-6.76 (m, 2H), 6.06 (ddt, J=17.3, 10.5, 5.3 Hz, 1H), 5.44 (dd, J=17.3, 1.5 Hz, 1H), 5.38-5.21 (m, 2H), 4.85 (d, J=3.8 Hz, 1H), 4.61 (dt, J=5.3, 1.6 Hz, 2H), 3.93 (d, J=12.4 Hz, 2H), 3.84-3.68 (m, 3H), 3.49 (s, 3H), 3.16 (s, 1H), 2.38 (d, J=10.5 Hz, 1H), 2.17 (s, 1H). ¹³C NMR (101 MHz, Chloroform-d) δ 167.99, 162.84, 132.44, 132.08, 121.84, 118.22, 114.43, 99.44, 77.50, 71.47, 70.93, 69.51, 68.90, 62.20, 55.48. ESI-MS: calcd for C₁₇H₂₂O₈Na [M+Na]⁺: 377.1212, found: 377.1194.

### Methyl-3-O-(4-(methylsulfonyl)benzoyl)-α-D-glucopyranoside (9)

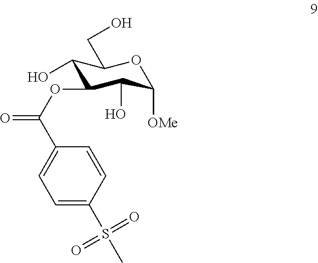

The product 9 (32.0 mg, 85%) was obtained as a white solid.

¹H NMR (400 MHz, Acetone-d₆) δ 8.32-8.27 (m, 2H), 8.13-8.07 (m, 2H), 5.46 (t, J=9.4 Hz, 1H), 4.78 (d, J=3.6 Hz, 1H), 3.98-3.61 (m, 5H), 3.45 (s, 3H), 3.20 (s, 3H). ¹³C NMR (101 MHz, Acetone-d₆) δ 164.74, 145.01, 135.25, 130.38, 127.41, 99.98, 77.90, 72.42, 70.71, 68.57, 61.42, 54.52, 43.22. ESI-MS: calcd for C₁₅H₂₀O₉SNa [M+Na]⁺: 399.0726, found: 399.0712.

### Methyl-3-O-(4-ethynylbenzoyl)-α-D-alucopyranoside (10)

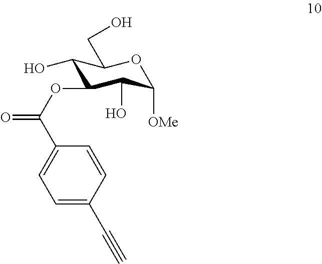

The product 10 (21.6 mg, 67%) was obtained as a white solid.

¹H NMR (400 MHz, Acetone-d₆) δ 8.07 (d, J=8.4 Hz, 2H), 7.63 (d, J=8.4 Hz, 2H), 5.43 (t, J=9.4 Hz, 1H), 4.77 (d, J=3.6 Hz, 1H), 3.91 (s, 1H), 3.85 (dd, J=11.6, 2.6 Hz, 1H), 3.76 (dd, J=10.2, 7.4 Hz, 2H), 3.69 (ddd, J=7.2, 4.7, 2.4 Hz, 2H), 3.44 (s, 3H). ¹³C NMR (101 MHz, Acetone-d₆) δ 165.30, 131.83, 130.97, 129.62, 126.64, 100.02, 82.52, 81.15, 77.36, 72.47, 70.82, 68.67, 61.50, 54.48. ESI-MS: calcd for C₁₆H₁₈O₇Na [M+Na]⁺: 345.0950, found: 345.0941.

### Methyl-3-O-(2-fluorobenzoyl)-α-D-glucopyranoside (11)

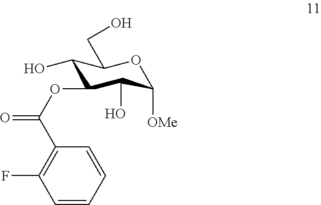

The product 11 (26.5 mg, 84%) was obtained as a white solid.

¹H NMR (400 MHz, Chloroform-d) δ 8.03 (td, J=7.5, 1.9 Hz, 1H), 7.67-7.51 (m, 1H), 7.27 (td, J=7.6, 1.1 Hz, 1H), 7.20 (ddd, J=10.9, 8.3, 1.1 Hz, 1H), 5.38 (t, J=9.4 Hz, 1H), 4.89 (d, J=3.8 Hz, 1H), 4.04-3.70 (m, 5H), 3.53 (s, 3H), 2.85 (s, 1H), 2.37 (d, J=10.6 Hz, 1H), 2.06 (s, 1H). ¹³C NMR (101 MHz, Chloroform-d) δ 165.89 (d, J=3.8 Hz), 162.10 (d, J=260.2 Hz), 135.04 (d, J=9.2 Hz), 132.45, 124.13 (d, J=3.8 Hz), 118.25 (d, J=9.6 Hz), 117.08 (d, J=22.4 Hz), 99.48, 78.14, 71.41, 70.99, 69.28, 62.19, 55.55. ESI-MS: calcd for C₁₄H₁₇O₇FNa [M+Na]⁺: 339.0856, found: 339.0843.

### Methyl-3-O-(3-chlorobenzoyl)-α-D-alucopyranoside (12)

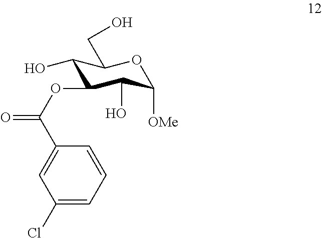

The product 12 (21.6 mg, 65%) was obtained as a white solid.

¹H NMR (400 MHz, Chloroform-d) δ 8.09 (t, J=1.9 Hz, 1H), 8.00 (dt, J=7.7, 1.4 Hz, 1H), 7.60 (ddd, J=7.9, 2.2, 1.1 Hz, 1H), 7.44 (t, J=7.9 Hz, 1H), 5.36 (t, J=9.4 Hz, 1H), 4.88 (d, J=3.8 Hz, 1H), 4.00-3.72 (m, 5H), 3.52 (s, 3H), 2.99 (s, 1H), 2.36 (d, J=11.0 Hz, 1H), 2.18 (s, 1H). ¹³C NMR (101 MHz, Chloroform-d) δ 166.71, 134.63, 133.47, 131.34, 129.97, 129.80, 128.11, 99.45, 77.90, 71.44, 70.94, 69.23, 62.10, 55.57. ESI-MS: calcd for C₁₄H₁₇O₇CINa [M+Na]⁺: 355.0561, found: 355.0554.

### Methyl-3-O-(3-bromobenzoyl)-α-D-glucopyranoside (13)

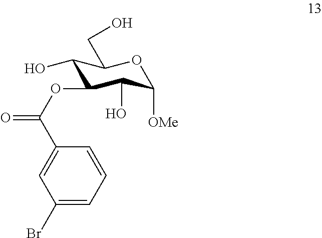

The product 13 (20.7 mg, 55%) was obtained as a white solid.

¹H NMR (400 MHz, Chloroform-d) δ 8.21 (s, 1H), 8.01 (d, J=7.8 Hz, 1H), 7.82-7.64 (m, 1H), 7.34 (t, J=7.9 Hz, 1H), 5.33 (t, J=9.4 Hz, 1H), 4.85 (d, J=3.8 Hz, 1H), 3.95-3.68 (m, 5H), 3.49 (s, 3H), 3.09 (s, 1H), 2.40 (d, J=11.0 Hz, 1H), 2.24 (s, 1H). ¹³C NMR (126 MHz, Chloroform-d) δ 166.53, 136.35, 132.83, 131.52, 130.03, 128.54, 122.50, 99.44, 77.82, 71.41, 70.91, 69.11, 62.03, 55.54. ESI-MS: calcd for C₁₄H₁₇O₇BrNa [M+Na]⁺: 399.0055, found: 399.0033.

### Methyl-3-O-(thiophene-2-carbonyl)-α-D-alucopyranoside (14)

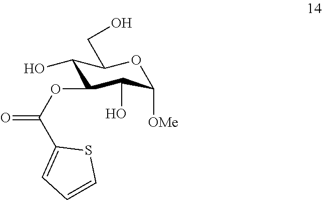

The product 14 (19.8 mg, 65%) was obtained as a white solid.

¹H NMR (400 MHz, Chloroform-d) δ 7.91 (dd, J=3.8, 1.3 Hz, 1H), 7.65 (dd, J=5.0, 1.3 Hz, 1H), 7.16 (dd, J=5.0, 3.8 Hz, 1H), 5.30 (t, J=9.3 Hz, 1H), 4.87 (d, J=3.8 Hz, 1H), 3.98-3.89 (m, 2H), 3.84 (t, J=9.4 Hz, 1H), 3.77 (dt, J=9.6, 3.8 Hz, 2H), 3.52 (s, 3H). ¹³C NMR (101 MHz, Chloroform-d) δ 163.50, 134.39, 133.30, 132.93, 127.96, 99.47, 77.86, 71.41, 70.90, 69.18, 62.13, 55.53. ESI-MS: calcd for C₁₂H₁₆O₇SNa [M+Na]⁺: 327.0514, found: 327.0511.

### Methyl-3-O-(5-nitrothiophene-2-formyl)-α-D-glucopyranoside (15)

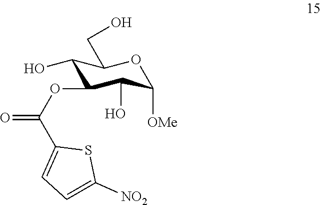

The product 15 (30.0 mg, 85%) was obtained as a yellow solid.

¹H NMR (400 MHz, Acetone-d₆) δ 8.08 (d, J=4.3 Hz, 1H), 7.85 (d, J=4.3 Hz, 1H), 5.38 (t, J=9.5 Hz, 1H), 4.78 (d, J=3.7 Hz, 1H), 4.74 (d, J=5.6 Hz, 1H), 4.02 (d, J=8.8 Hz, 1H), 3.85 (ddd, J=11.6, 5.9, 2.6 Hz, 1H), 3.81-3.61 (m, 5H), 3.44 (s, 3H). ¹³C NMR (101 MHz, Acetone-d₆) δ 160.27, 155.00, 139.15, 132.07, 128.79, 99.91, 78.91, 72.37, 70.51, 68.37, 61.36, 54.51. ESI-MS: calcd for C₁₂H₁₅O₉NSNa [M+Na]⁺: 372.0365, found: 372.0368.

### Methyl-3-O-(5-methylfuran-2-carbonyl)-α-D-glucopyranoside (16)

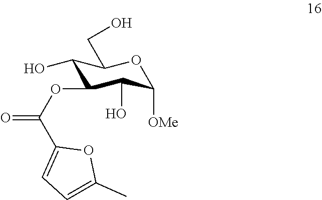

The product 16 (20.2 mg, 67%) was obtained as a white solid.

¹H NMR (500 MHz, Chloroform-d) δ 7.20 (d, J=3.5 Hz, 1H), 6.17 (dd, J=3.4, 1.0 Hz, 1H), 5.26 (t, J=9.4 Hz, 1H), 4.85 (d, J=3.8 Hz, 1H), 3.91 (qd, J=11.8, 3.8 Hz, 2H), 3.83-3.71 (m, 3H), 3.50 (s, 3H), 2.93 (s, 1H), 2.41 (s, 3H), 2.38-2.29 (m, 1H), 2.11 (s, 1H). ¹³C NMR (126 MHz, Chloroform-d) δ 159.97, 158.07, 142.29, 120.83, 108.77, 99.42, 77.36, 71.40, 70.85, 69.25, 62.17, 55.49, 14.05. ESI-MS: calcd for C₁₃H₁₃O₈Na [M+Na]⁺: 325.0899, found: 325.0882.

### Methyl-3-O-(2-naphthoyl)-α-D-glucopyranoside (17)

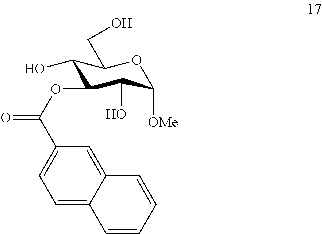

The product 17 (22.3 mg, 64%) was obtained as a white solid.

¹H NMR (400 MHz, Chloroform-d) δ 8.80-8.64 (m, 1H), 8.11 (dd, J=8.6, 1.7 Hz, 1H), 8.03-7.95 (m, 1H), 7.91 (d, J=8.3 Hz, 2H), 7.62 (dddd, J=22.7, 8.1, 6.8, 1.3 Hz, 2H), 5.42 (t, J=9.5 Hz, 1H), 4.91 (d, J=3.8 Hz, 1H), 4.09-3.73 (m, 5H), 3.53 (s, 3H), 3.09 (s, 1H), 2.41 (s, 1H), 2.15 (s, 1H). ¹³C NMR (101 MHz, Chloroform-d) δ 168.34, 135.79, 132.44, 131.68, 129.47, 128.56, 128.29, 127.81, 126.79, 126.69, 125.32, 99.50, 77.86, 71.50, 71.01, 69.51, 62.22, 55.55. ESI-MS: calcd for C₁₃H₂₁O₇[M+H]⁺: 349.1287, found: 349.1281.

### Methyl-3-O-(3-phenylpropioloyl)-α-D-glucopyranoside (18)

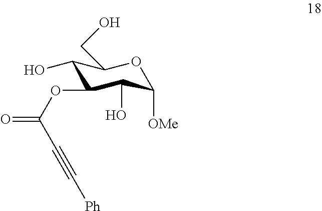

The product 18 (21.6 mg, 67%) was obtained as a white solid.

¹H NMR (400 MHz, Chloroform-d) δ 7.71-7.56 (m, 2H), 7.50-7.43 (m, 1H), 7.38 (dd, J=8.2, 6.7 Hz, 2H), 5.24 (t, J=9.4 Hz, 1H), 4.82 (d, J=3.8 Hz, 1H), 3.89 (t, J=3.4 Hz, 2H), 3.78 (t, J=9.4 Hz, 1H), 3.73-3.65 (m, 2H), 3.47 (s, 3H). ¹³C NMR (101 MHz, Chloroform-d) δ 153.74, 131.98, 129.79, 127.53, 118.29, 98.31, 87.23, 79.11, 77.19, 70.21, 69.72, 67.80, 60.98, 54.46. ESI-MS: calcd for C₁₆H₁₃O₇Na [M+Na]⁺: 345.0950, found: 345.0945.

### Methyl-3-O-cinnamoyl-α-D-glucopyranoside (19)

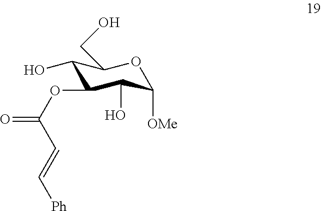

The product 19 (26.6 mg, 82%) was obtained as a white solid.

¹H NMR (400 MHz, Chloroform-d) δ 7.80 (d, J=16.0 Hz, 1H), 7.67-7.53 (m, 2H), 7.43 (dd, J=5.1, 1.9 Hz, 3H), 6.56 (d, J=16.0 Hz, 1H), 5.24 (t, J=9.4 Hz, 1H), 4.87 (d, J=3.9 Hz, 1H), 3.91 (d, J=19.1 Hz, 2H), 3.76 (dq, J=9.8, 6.2, 4.8 Hz, 3H), 3.52 (s, 3H), 3.13 (s, 1H), 2.41 (d, J=10.7 Hz, 1H), 2.21 (s, 1H). ¹³C NMR (101 MHz, Chloroform-d) δ 168.63, 146.42, 134.16, 130.66, 128.96, 128.30, 117.28, 99.44, 77.24, 71.48, 70.92, 69.49, 62.22, 55.51. ESI-MS: calcd for C₁₆H₂₀O₇Na [M+Na]⁺: 347.1107, found: 347.1101.

### Methyl-3-O-(4-chlorobenzoyl)-β-D-glucopyranoside (20)

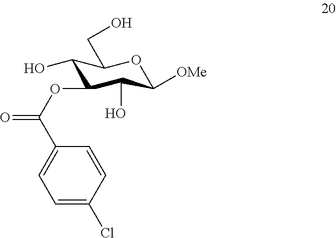

The product 20 (21.9 mg, 66%) was obtained as a white solid.

¹H NMR (400 MHz, Acetone-d₆) δ 8.14-7.97 (m, 2H), 7.69-7.49 (m, 2H), 5.24 (t, J=9.4 Hz, 1H), 4.64 (d, J=5.7 Hz, 1H), 4.52 (d, J=4.6 Hz, 1H), 4.38 (d, J=7.8 Hz, 1H), 3.98-3.82 (m, 1H), 3.79-3.67 (m, 3H), 3.52-3.47 (m, 5H). ¹³C NMR (101 MHz, Acetone-d₆) δ 164.79, 138.51, 131.26, 129.64, 128.57, 104.10, 79.09, 76.45, 72.17, 68.87, 61.67, 56.01. ESI-MS: calcd for C₁₄H₁₇O₇CINa [M+Na]⁺: 355.0561, found: 355.0543.

### Phenyl-3-O-(4-chlorobenzoyl)-α-D-glucopyranoside (21)

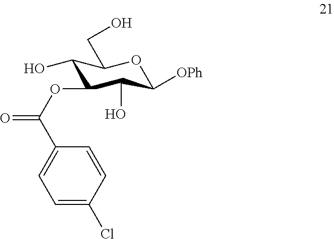

The product 21 (29.1 mg, 74%) was obtained as a white solid.

Retention factor (R_{F}, hexane:ethyl acetate=1:1): 0.30. ¹H NMR (400 MHz, Acetone-d₆) δ 8.18-7.97 (m, 2H), 7.69-7.51 (m, 2H), 7.42-7.26 (m, 2H), 7.19-7.08 (m, 2H), 7.04 (tt, J=7.3, 1.1 Hz, 1H), 5.39 (t, J=9.4 Hz, 1H), 5.20 (d, J=7.7 Hz, 1H), 4.02-3.67 (m, 5H). ¹³C NMR (101 MHz, Acetone-d₆) δ 164.79, 157.84, 138.59, 131.31, 129.61, 129.34, 128.61, 122.09, 116.52, 100.83, 78.92, 76.70, 72.05, 68.48, 61.33. ESI-MS: calcd for C₁₉H₁₉O₇CINa [M+Na]⁺: 417.0717, found: 417.0695.

### Octyl-3-O-(4-chlorobenzoyl)-α-D-glucopyranoside (22)

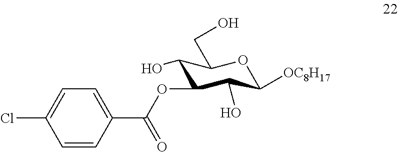

The product 22 (32.2 mg, 75%) was obtained as a white solid.

¹H NMR (400 MHz, Chloroform-d) δ 8.04 (d, J=8.6 Hz, 2H), 7.45 (d, J=8.6 Hz, 2H), 5.19 (t, J=9.4 Hz, 1H), 4.46 (d, J=7.8 Hz, 1H), 4.15-3.77 (m, 4H), 3.73-3.55 (m, 2H), 3.51 (dt, J=9.6, 4.0 Hz, 1H), 1.65 (q, J=6.9 Hz, 2H), 1.41-1.15 (m, 10H), 0.90 (t, J=6.6 Hz, 3H). ¹³C NMR (101 MHz, Chloroform-d) δ 166.98, 140.11, 131.37, 128.87, 127.83, 102.79, 78.93, 75.67, 72.26, 70.61, 69.74, 62.32, 31.79, 29.60, 29.35, 29.22, 25.93, 22.64, 14.09. ESI-MS: calcd for C₂₁H₃₁O₇ CINa [M+Na]⁺: 453.1656, found: 453.1650.

### 3-O-(4-chlorobenzoyl)-geniposide (23)

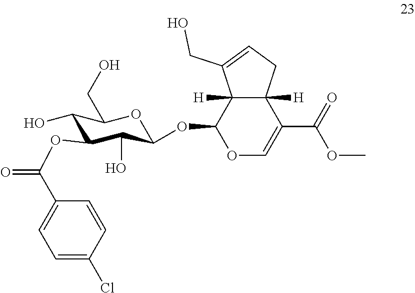

The product 23 (37.9 mg, 72%) was obtained as a white solid.

¹H NMR (400 MHz, Acetone-d₆) δ 8.22-7.96 (m, 2H), 7.65-7.50 (m, 2H), 5.80 (s, 1H), 5.30 (t, J=9.4 Hz, 1H), 5.22 (d, J=7.3 Hz, 1H), 4.94 (d, J=7.8 Hz, 1H), 4.71 (dd, J=10.4, 5.3 Hz, 2H), 4.37 (d, J=14.7 Hz, 1H), 4.18 (d, J=14.3 Hz, 1H), 3.99-3.72 (m, 5H), 3.68 (s, 3H), 3.62 (ddd, J=9.5, 7.9, 4.8 Hz, 1H), 3.55 (ddd, J=9.8, 4.9, 2.5 Hz, 1H), 3.17 (qd, J=7.9, 1.3 Hz, 1H), 2.79 (dd, J=16.4, 8.4 Hz, 2H), 2.70 (t, J=7.6 Hz, 1H), 2.12 (dt, J=7.7, 2.4 Hz, 1H). ¹³C NMR (101 MHz, Acetone-d₆) δ 166.93, 164.75, 151.38, 144.51, 138.57, 131.29, 129.58, 128.59, 126.09, 111.50, 99.51, 97.21, 78.77, 76.75, 72.03, 68.49, 61.27, 60.21, 50.39, 46.07, 38.42, 35.21. ESIMS: calcd for C₂₄H₂₇O₁₁CINa [M+Na]⁺: 549.1140, found: 549.1141.

### 3-O-(4-chlorobenzoyl)-dapagPifGozin (24)

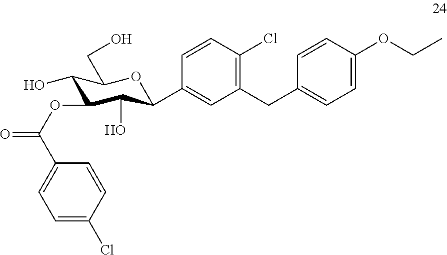

The product 24 (38.8 mg, 71%) was obtained as a white solid.

¹H NMR (400 MHz, Acetone-d₆) δ 8.16-7.92 (m, 2H), 7.62-7.51 (m, 2H), 7.46 (s, 1H), 7.38 (s, 2H), 7.15 (d, J=8.6 Hz, 2H), 6.83 (d, J=8.6 Hz, 2H), 5.36 (t, J=9.2 Hz, 1H), 4.37 (d, J=9.4 Hz, 1H), 4.12-3.96 (m, 4H), 3.94-3.85 (m, 2H), 3.78 (dd, J=11.9, 5.0 Hz, 1H), 3.72-3.64 (m, 1H), 3.61 (ddd, J=9.5, 4.9, 2.6 Hz, 1H), 1.34 (t, J=7.0 Hz, 3H). ¹³C NMR (101 MHz, Acetone-d₆) δ 165.03, 157.57, 139.01, 138.49, 138.36, 132.85, 131.40, 131.28, 130.91, 129.71, 129.68, 128.89, 128.53, 127.08, 114.27, 81.21, 80.89, 80.80, 73.52, 68.91, 62.98, 61.81, 38.01, 14.25. ESI-MS: calcd for C₂₈H₂₉O₇C₁₂ [M+H]⁺: 547.1290, found: 547.1295.

### 3-O-(4-chlorobenzoyl)-empaaliflozin (25)

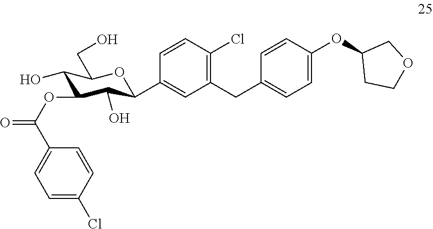

The product 25 (45.3 mg, 77%) was obtained as a white solid.

¹H NMR (400 MHz, Acetone-d₆) δ 8.15-7.86 (m, 2H), 7.62-7.52 (m, 2H), 7.47 (s, 1H), 7.38 (s, 2H), 7.29-7.02 (m, 2H), 6.94-6.70 (m, 2H), 5.36 (t, J=9.2 Hz, 1H), 4.99 (td, J=4.5, 2.3 Hz, 1H), 4.67 (d, J=5.7 Hz, 1H), 4.48 (d, J=6.1 Hz, 1H), 4.38 (d, J=9.4 Hz, 1H), 4.13-4.00 (m, 2H), 3.97-3.85 (m, 4H), 3.80 (tdd, J=11.1, 5.1, 2.8 Hz, 3H), 3.73-3.58 (m, 3H), 2.23 (dtd, J=14.3, 8.2, 6.2 Hz, 1H). ¹³C NMR (101 MHz, Acetone-d₆) δ 165.02, 156.12, 139.04, 138.50, 138.27, 132.85, 131.81, 131.28, 130.92, 129.80, 129.71, 128.90, 128.54, 127.13, 115.22, 81.21, 80.91, 80.80, 77.35, 73.55, 72.60, 68.95, 66.58, 61.86, 37.99, 32.82. ESI-MS: calcd for C₃₀H₃₁O₈C₁₂ [M+H]⁺: 589.1396, found: 589.1387.

### Methyl-3-O-(4-chlorobenzoyl)-α-D-galactopyranoside (26)

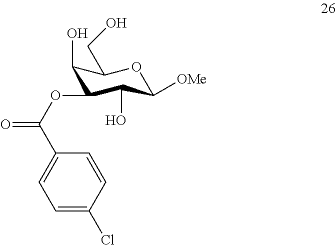

The product 26 (20.2 mg, 61%) was obtained as a white solid.

¹H NMR (500 MHz, Acetone-d₆) δ 8.09 (d, J=8.6 Hz, 2H), 7.67-7.46 (m, 2H), 5.00 (dd, J=10.0, 3.3 Hz, 1H), 4.34 (d, J=7.7 Hz, 1H), 4.24 (d, J=3.3 Hz, 1H), 3.96 (dd, J=10.0, 7.7 Hz, 1H), 3.81 (dd, J=6.0, 1.8 Hz, 2H), 3.72-3.64 (m, 1H), 3.51 (s, 3H). ¹³C NMR (101 MHz, Acetone-d₆) δ 164.82, 138.68, 131.32, 129.42, 128.59, 104.77, 77.55, 74.88, 68.63, 66.75, 61.08, 55.85. ESI-MS: calcd for C₁₄H₁₇O₇ CINa [M+Na]⁺: 355.0561, found: 355.0545.

### Methyl-3-O-(4-chlorobenzoyl)-α-D-galactopyranoside (27)

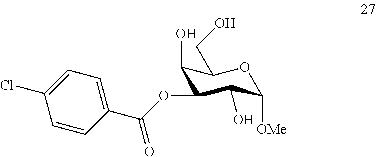

The product 27 (16.6 mg, 50%) was obtained as a white solid.

¹H NMR (400 MHz, Acetone-d₆) δ 8.14-7.93 (m, 2H), 7.65-7.42 (m, 2H), 5.22 (dd, J=10.4, 3.1 Hz, 1H), 4.80 (d, J=3.8 Hz, 1H), 4.30 (dd, J=3.2, 1.3 Hz, 1H), 4.22 (dd, J=10.4, 3.8 Hz, 1H), 3.93-3.84 (m, 1H), 3.84-3.71 (m, 2H), 3.43 (s, 3H). ¹³C NMR (101 MHz, Acetone-d₆) δ 165.04, 138.66, 131.33, 129.48, 128.58, 100.43, 75.10, 70.77, 67.54, 66.68, 61.32, 54.53. ESI-MS: calcd for C₁₄H₁₇O₇CINa [M+Na]⁺: 355.0561, found: 355.0543.

### Isopropylthio-3-O-(4-chlorobenzoyl)-α-D-galactopyranoside (28)

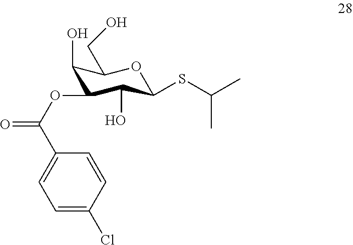

The product 28 (23.3 mg, 62%) was obtained as a white solid.

¹H NMR (400 MHz, Chloroform-d) δ 8.11-7.87 (m, 2H), 7.53-7.38 (m, 2H), 5.12 (dd, J=9.6, 3.2 Hz, 1H), 4.57 (d, J=9.7 Hz, 1H), 4.34 (d, J=3.1 Hz, 1H), 4.14-3.85 (m, 3H), 3.71 (t, J=5.0 Hz, 1H), 3.28 (p, J=6.8 Hz, 1H), 1.39 (dd, J=6.7, 2.8 Hz, 6H). ¹³C NMR (101 MHz, Chloroform-d) δ 165.30, 139.94, 131.31, 128.84, 128.05, 86.83, 77.86, 76.93, 68.80, 67.88, 63.02, 36.11, 24.22, 24.08. ESI-MS: calcd for C₁₆H₂₁O₆CISNa [M+Na]⁺: 399.0645, found: 399.0625.

### Phenyl-3-O-(4-chlorobenzoyl)-α-D-galactopyranoside (29)

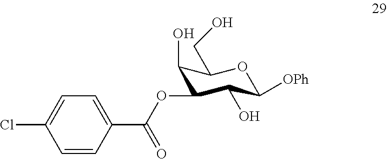

The product 29 (24.4 mg, 62%) was obtained as a white solid.

¹H NMR (400 MHz, Methanol-d₄) δ 8.20-8.06 (m, 2H), 7.59-7.49 (m, 2H), 7.36-7.27 (m, 2H), 7.20-7.12 (m, 2H), 7.04 (td, J=7.3, 1.1 Hz, 1H), 5.10 (dd, J=10.1, 3.3 Hz, 1H), 5.06 (d, J=7.7 Hz, 1H), 4.27-4.24 (m, 1H), 4.20 (dd, J=10.1, 7.7 Hz, 1H), 3.89-3.74 (m, 3H). ¹³C NMR (101 MHz, Methanol-d₄) δ 165.32, 157.77, 139.18, 131.07, 129.02, 128.83, 128.38, 122.07, 116.48, 101.54, 76.70, 75.27, 68.50, 66.42, 60.71. ESI-MS: calcd for C₁₉H₁₉O₇CINa [M+Na]⁺: 417.0717, found: 417.0698.

### (4-methoxyphenyl)-3-O-(4-chlorobenzoyl)-α-D-galactopyranoside (30)

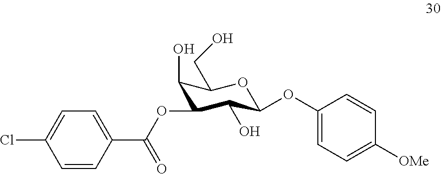

The product 30 (22.5 mg, 53%) was obtained as a white solid.

¹H NMR (400 MHz, Methanol-d₄) δ 8.23-8.01 (m, 2H), 7.69-7.43 (m, 2H), 7.19-7.05 (m, 2H), 6.97-6.76 (m, 2H), 5.08 (dd, J=10.1, 3.3 Hz, 1H), 4.93 (d, J=7.8 Hz, 1H), 4.24 (d, J=3.3 Hz, 1H), 4.16 (dd, J=10.2, 7.8 Hz, 1H), 3.86-3.73 (m, 3H), 3.78 (s, 3H). ¹³C NMR (101 MHz, Methanol-d₄) δ 165.32, 155.34, 151.82, 139.17, 131.07, 128.83, 128.37, 117.97, 114.06, 102.70, 76.71, 75.22, 68.54, 66.42, 60.71, 54.65. ESI-MS: calcd for C₂₀H₂₁O₈ CINa [M+Na]⁺: 447.0823, found: 447.0816.

### 4-Nitrophenyl-3-O-(4-chlorobenzoyl)-α-D-galactopyranoside (31)

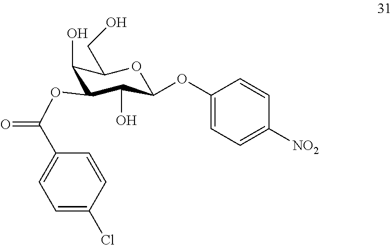

The product 31 (29.9 mg, 68%) was obtained as a white solid.

¹H NMR (400 MHz, Acetone-d₆) δ 8.33-8.21 (m, 2H), 8.19-8.06 (m, 2H), 7.69-7.51 (m, 2H), 7.41-7.24 (m, 2H), 5.39 (d, J=7.6 Hz, 1H), 5.18 (dd, J=10.0, 3.2 Hz, 1H), 5.07 (d, J=4.8 Hz, 1H), 4.60 (d, J=5.1 Hz, 1H), 4.36 (ddd, J=9.9, 7.6, 4.5 Hz, 2H), 4.05 (dt, J=18.7, 5.9 Hz, 2H), 3.86 (t, J=5.7 Hz, 2H). ¹³C NMR (101 MHz, Acetone-d₆) δ 164.79, 162.64, 142.47, 138.82, 131.37, 129.25, 128.65, 125.53, 116.65, 100.96, 77.14, 75.57, 68.29, 66.54, 60.98. ESIMS: calcd for C₁₉H₁₃O₉CINNa [M+Na]⁺: 462.0568, found: 462.0559.

### Allyl-3-O-(4-chlorobenzoyl)-α-D-galactopyranoside (32)

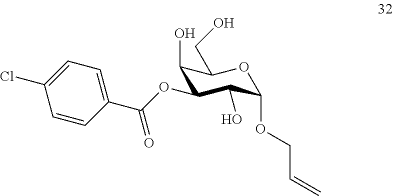

The product 32 (19.0 mg, 53%) was obtained as a white solid.

¹H NMR (400 MHz, Acetone-d₆) δ 8.22-7.94 (m, 2H), 7.69-7.43 (m, 2H), 6.13-5.86 (m, 1H), 5.39 (dq, J=17.3, 1.8 Hz, 1H), 5.27 (dd, J=10.5, 3.1 Hz, 1H), 5.18 (dq, J=10.4, 1.5 Hz, 1H), 4.97 (d, J=3.8 Hz, 1H), 4.45 (s, 1H), 4.36-4.18 (m, 3H), 4.09 (ddt, J=13.2, 5.8, 1.5 Hz, 1H), 3.96 (td, J=5.9, 1.4 Hz, 1H), 3.87-3.68 (m, 4H). ¹³C NMR (101 MHz, Acetone-d₆) δ 165.05, 138.66, 134.67, 131.33, 129.46, 128.58, 116.08, 98.62, 75.13, 71.01, 68.02, 67.55, 66.66, 61.30. ESI-MS: calcd for C₁₆H₁₉O₇CINa [M+Na]⁺: 381.0717, found: 381.0704.

### Methyl 2-(benzyloxy)carbonyl)amino-2-deoxy-3-O-(4-chlorobenzoyl)-α-D-glucopyranoside (33)

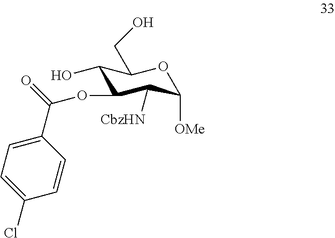

The product 33 (32.5 mg, 70%) was obtained as a white solid.

¹H NMR (400 MHz, Chloroform-d) δ 7.96 (d, J=8.6 Hz, 2H), 7.38 (d, J=8.6 Hz, 2H), 7.26-7.12 (m, 5H), 5.27 (t, J=9.9 Hz, 1H), 5.18 (d, J=10.1 Hz, 1H), 5.06-4.89 (m, 2H), 4.79 (d, J=3.6 Hz, 1H), 4.16 (td, J=10.4, 3.8 Hz, 1H), 3.94-3.84 (m, 3H), 3.75 (dt, J=9.8, 3.8 Hz, 1H), 3.43 (s, 3H). ¹³C NMR (101 MHz, Chloroform-d) δ 167.13, 155.96, 140.00, 136.03, 131.44, 128.81, 128.39, 128.10, 127.76, 127.65, 98.73, 76.02, 71.65, 69.70, 66.88, 62.11, 55.35, 53.44. ESI-MS: calcd for C₂₂H₂₄O₈CINNa [M+Na]⁺: 488.1088, found: 488.1070.

### Methyl-3-O-(4-chlorobenzoyl)-α-D-xylopyranoside (34)

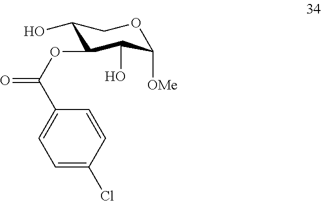

The product 34 (21.7 mg, 72%) was obtained as a white solid.

¹H NMR (400 MHz, Acetone-d₆) δ 8.15-7.95 (m, 2H), 7.73-7.40 (m, 2H), 5.34 (t, J=9.4 Hz, 1H), 4.72 (d, J=3.5 Hz, 1H), 4.53 (d, J=5.6 Hz, 1H), 3.92-3.82 (m, 1H), 3.79 (d, J=9.0 Hz, 1H), 3.67 (dd, J=11.0, 5.7 Hz, 2H), 3.58 (t, J=10.8 Hz, 1H), 3.43 (s, 3H). ¹³C NMR (101 MHz, Acetone-d₆) δ 165.06, 138.53, 131.22, 129.63, 128.57, 100.24, 77.40, 70.71, 68.32, 61.76, 54.61. ESI-MS: calcd for C₁₃H₁₅O₆CINa [M+Na]⁺: 325.0455, found: 325.0440.

### Methyl-3-O-(4-chlorobenzoyl)-β-D-xylopyranoside (35)

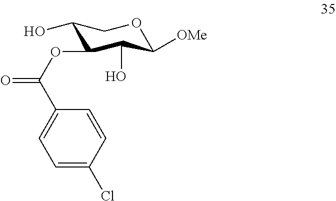

The product 35 (19.9 mg, 66%) was obtained as a white solid.

¹H NMR (400 MHz, Chloroform-d) δ 8.21-7.95 (m, 2H), 7.61-7.43 (m, 2H), 5.13 (t, J=8.1 Hz, 1H), 4.40 (d, J=6.4 Hz, 1H), 4.17 (dd, J=11.9, 4.9 Hz, 1H), 3.96 (td, J=8.4, 4.8 Hz, 1H), 3.71 (dd, J=8.3, 6.5 Hz, 1H), 3.60 (s, 3H), 3.48 (dd, J=11.9, 8.7 Hz, 1H). ¹³C NMR (101 MHz, Chloroform-d) δ 166.75, 140.15, 131.40, 128.90, 127.85, 103.79, 77.59, 71.09, 68.74, 64.87, 57.04. ESI-MS: calcd for C₁₃H₁₅O₆CINa [M+Na]⁺: 325.0455, found: 325.0438.

### Methyl-3-O-(4-chlorobenzoyl)-α-D-mannopyranoside (36)

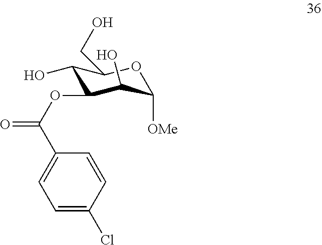

The product 36 (20.5 mg) was obtained as a white solid (total acylates 24.6 mg, 74%).

¹H NMR (400 MHz, Acetone-d₆) δ 8.09 (d, J=8.4 Hz, 2H), 7.56 (d, J=8.1 Hz, 2H), 5.23 (dd, J=9.8, 3.2 Hz, 1H), 4.72 (s, 1H), 4.54 (d, J=5.5 Hz, 2H), 4.22-4.08 (m, 2H), 3.93-3.84 (m, 1H), 3.79 (p, J=5.8, 5.1 Hz, 1H), 3.71-3.59 (m, 2H), 3.41 (s, 3H). ¹³C NMR (101 MHz, Acetone-d₆) δ 164.86, 138.63, 131.36, 129.47, 128.53, 101.41, 76.21, 73.45, 68.54, 64.90, 61.88, 53.98. ESI-MS: calcd for C₁₄H₁₇O₇ CINa [M+Na]⁺: 355.0561, found: 355.0543.

### Methyl-3-O-(4-chlorobenzoyl)-α-L-Rhamnopyranoside (37)

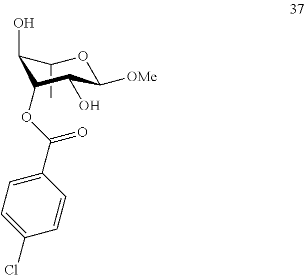

The product 37 (27.5 mg, 87%) was obtained as a white solid.

¹H NMR (400 MHz, Chloroform-d) δ 8.18-7.86 (m, 2H), 7.58-7.41 (m, 2H), 5.33-5.24 (m, 1H), 4.76 (d, J=1.9 Hz, 1H), 4.19 (dd, J=3.2, 1.8 Hz, 1H), 3.96-3.68 (m, 2H), 3.47 (s, 3H), 1.44 (d, J=5.9 Hz, 3H). ¹³C NMR (101 MHz, Chloroform-d) δ 166.10, 140.07, 131.28, 128.90, 128.04, 100.64, 75.74, 71.50, 69.74, 68.49, 55.07, 17.63. ESI-MS: calcd for C₁₄H₁₇O₆CINa [M+Na]⁺: 339.0611, found: 339.0598.

### 3-O-(4-chlorobenzoyl)-D-glucal (38)

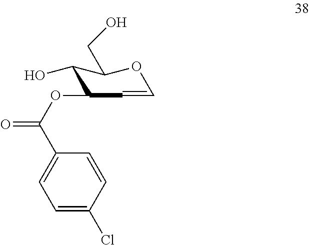

The product 38 (19.0 mg, 67%) was obtained as a white solid.

¹H NMR (500 MHz, Acetone-d₆) δ 8.06 (d, J=8.3 Hz, 2H), 7.70-7.37 (m, 2H), 6.52 (d, J=5.9 Hz, 1H), 5.53 (d, J=6.9 Hz, 1H), 4.82 (dd, J=6.1, 2.5 Hz, 1H), 4.17 (dd, J=9.3, 6.9 Hz, 1H), 4.05-3.82 (m, 3H). ¹³C NMR (126 MHz, Acetone-d₆) δ 165.16, 146.04, 138.80, 131.17, 129.29, 128.73, 98.91, 79.34, 73.45, 66.18, 60.61. ESI-MS: calcd for C₁₃H₁₃O₅CINa [M+Na]⁺: 307.0349, found: 307.0339.

### Methyl-2-O-(4-chlorobenzoyl)-α-D-galactopyranoside (40)

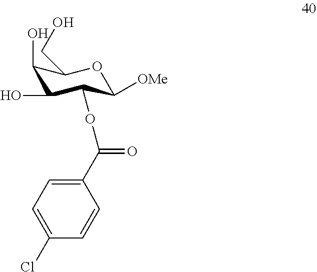

The product 40 (20.0 mg) was obtained as a white solid (total acylates 23.9 mg, 72%).

¹H NMR (400 MHz, Acetone-d₆) δ 8.19-7.90 (m, 2H), 7.67-7.47 (m, 2H), 5.29 (dd, J=9.8, 8.0 Hz, 1H), 4.53 (d, J=8.0 Hz, 1H), 4.04 (dd, J=3.4, 1.2 Hz, 1H), 3.90 (dd, J=9.8, 3.4 Hz, 1H), 3.83 (d, J=5.9 Hz, 2H), 3.67 (td, J=6.0, 1.2 Hz, 1H), 3.42 (s, 3H). ¹³C NMR (101 MHz, Acetone-d₆) δ 164.47, 138.64, 131.16, 129.48, 128.68, 101.97, 75.41, 73.59, 71.97, 69.31, 61.22, 55.44. ESI-MS: calcd for C₁₄H₁₇O₇CINa [M+Na]⁺: 355.0561, found: 355.0545.

### (4-methoxyphenyl)-2-O-(4-chlorobenzoyl)-β-D-galactopyranoside (41)

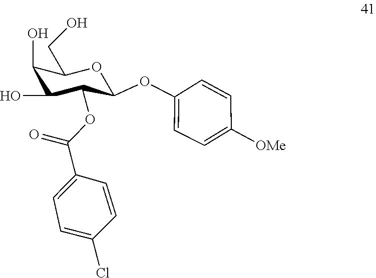

The product 41 (25.9 mg, 61%) was obtained as a white solid.

¹H NMR (400 MHz, Acetone-d₆) δ 8.07 (d, J=8.6 Hz, 2H), 7.57 (d, J=8.6 Hz, 2H), 7.00-6.86 (m, 2H), 6.84-6.70 (m, 2H), 5.54 (dd, J=9.8, 8.0 Hz, 1H), 5.13 (d, J=8.0 Hz, 1H), 4.34 (d, J=7.2 Hz, 1H), 4.22 (s, 1H), 4.10 (d, J=3.5 Hz, 1H), 4.01 (td, J=12.1, 11.0, 4.5 Hz, 2H), 3.86 (dt, J=4.1, 2.3 Hz, 3H), 3.73 (s, 3H). ¹³C NMR (101 MHz, Acetone-d₆) δ 164.52, 155.35, 151.73, 138.74, 131.17, 129.32, 128.73, 118.16, 114.35, 100.74, 75.77, 73.58, 71.88, 69.29, 61.28, 54.90. ESI-MS: calcd for C₂₀H₂₁O₈ CINa [M+Na]⁺: 447.0823, found: 447.0821.

### 4-Nitrophenyl-2-O-(4-chlorobenzoyl)-β-D-galactopyranoside (42)

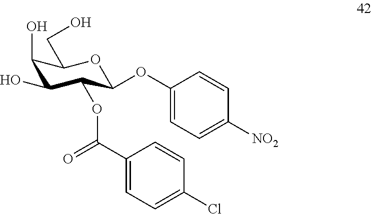

The product 42 (25.5 mg, 58%) was obtained as a white solid.

¹H NMR (400 MHz, DMSO-d₆) δ 8.17 (d, J=8.8 Hz, 2H), 7.97 (d, J=8.2 Hz, 2H), 7.60 (d, J=8.3 Hz, 2H), 7.15 (d, J=8.9 Hz, 2H), 5.51 (d, J=8.0 Hz, 1H), 5.40 (t, J=8.7 Hz, 1H), 5.33 (s, 1H), 5.04 (d, J=4.3 Hz, 1H), 4.81 (s, 1H), 3.92-3.77 (m, 3H), 3.67-3.52 (m, 2H). ¹³C NMR (101 MHz, DMSO-d₆) δ 164.74, 162.17, 142.47, 138.80, 131.60, 129.38, 129.00, 126.25, 117.06, 98.44, 76.58, 73.27, 71.31, 68.63, 60.55. ESI-MS: calcd for C₁₉H₁₃O₉NCINa [M+Na]⁺: 462.0568, found: 462.0573.

### Isopropylthio-2-O-(4-chlorobenzoyl)-β-D-galactopyranoside (43)

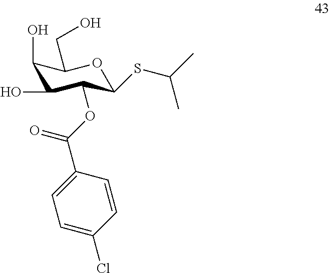

The product 43 (27.0 mg, 72%) was obtained as a white solid.

¹H NMR (400 MHz, Acetone-de) δ 8.11-7.92 (m, 2H), 7.62-7.49 (m, 2H), 5.32 (t, J=9.7 Hz, 1H), 4.79 (d, J=10.0 Hz, 1H), 4.10 (dd, J=3.4, 1.2 Hz, 1H), 3.95 (dd, J=9.3, 3.4 Hz, 1H), 3.81 (d, J=6.3 Hz, 2H), 3.72 (ddd, J=6.4, 5.3, 1.2 Hz, 1H), 3.22 (p, J=6.8 Hz, 1H), 1.24 (dd, J=13.1, 6.8 Hz, 6H). ¹³C NMR (101 MHz, Acetone-de) δ 164.50, 138.68, 131.20, 129.49, 128.68, 83.01, 79.27, 73.01, 72.39, 69.44, 61.41, 34.39, 23.73, 23.26. ESI-MS: calcd for C₁₆H₂₁O₆CISNa [M+Na]⁺: 399.0645, found: 399.0632.

### Isopropylthio-2-O-cinnamon acyl-β-D-galactopyranoside (44)

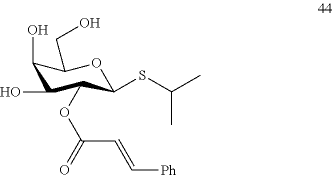

The product 44 (28.3 mg, 77%) was obtained as a white solid.

¹H NMR (400 MHz, Acetone-de) 6 7.80-7.57 (m, 3H), 7.46 (dd, J=5.0, 2.0 Hz, 3H), 6.57 (d, J=16.1 Hz, 1H), 5.21 (t, J=9.7 Hz, 1H), 4.68 (d, J=10.0 Hz, 1H), 4.24 (s, 1H), 4.07 (t, J=6.7 Hz, 2H), 3.81 (dd, J=20.9, 7.5 Hz, 4H), 3.72-3.60 (m, 1H), 3.22 (p, J=6.8 Hz, 1H), 1.26 (dd, J=15.6, 6.7 Hz, 6H). ¹³C NMR (126 MHz, Acetone-d₆) δ 165.60, 144.53, 134.56, 130.30, 128.96, 128.15, 118.41, 83.15, 79.18, 73.12, 71.46, 69.44, 61.45, 34.32, 23.72, 23.32. ESI-MS: calcd for C₁₈H₂₄O₆SNa [M+Na]⁺: 391.1191, found: 391.1191.

### Phenyl-2-O-(4-chlorobenzoyl)-β-D-galactopyranoside (45)

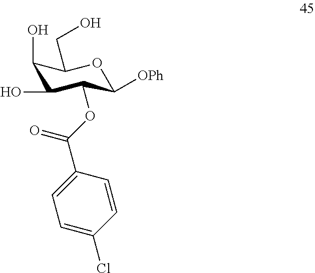

The product 45 (28.0 mg, 71%) was obtained as a white solid.

¹H NMR (400 MHz, Acetone-d₆) δ 8.15-7.88 (m, 2H), 7.66-7.40 (m, 2H), 7.35-7.20 (m, 2H), 7.09-6.91 (m, 3H), 5.59 (dd, J=9.8, 8.0 Hz, 1H), 5.29 (d, J=8.0 Hz, 1H), 4.14 (d, J=3.4 Hz, 1H), 4.07 (dd, J=9.8, 3.4 Hz, 1H), 3.95-3.77 (m, 3H). ¹³C NMR (101 MHz, Acetone-d₆) δ 164.52, 157.73, 138.75, 131.17, 129.36, 129.27, 128.72, 122.33, 116.67, 99.58, 75.85, 73.48, 71.85, 69.26, 61.23. ESI-MS: calcd for C₁₉H₁₉O₇CINa [M+Na]⁺: 417.0717, found: 417.0708.

### Phenyl-2-O-(4-methoxycarbonyl benzoyl)-α-D-galactopyranoside (46)

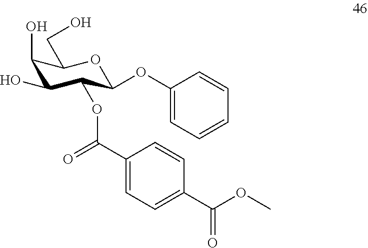

The product 46 (29.3 mg, 70%) was obtained as a white solid.

¹H NMR (400 MHz, Acetone-d₆) δ 8.27-8.04 (m, 4H), 7.24 (dd, J=8.8, 7.2 Hz, 2H), 7.09-6.84 (m, 3H), 5.62 (dd, J=9.8, 8.0 Hz, 1H), 5.31 (d, J=8.0 Hz, 1H), 4.44 (s, 1H), 4.28 (s, 1H), 4.14 (d, J=3.4 Hz, 1H), 4.09 (dd, J=9.7, 3.4 Hz, 2H), 3.96-3.92 (m, 4H), 3.88 (d, J=5.5 Hz, 2H). ¹³C NMR (101 MHz, Acetone-d₆) δ 165.62, 164.64, 157.70, 134.30, 134.06, 129.59, 129.37, 122.34, 116.65, 99.54, 75.86, 73.66, 71.83, 69.28, 61.24, 51.83. ESI-MS: calcd for C₂₁H₂₂O₉Na [M+Na]⁺: 441.1162, found: 441.1163.

### Methyl-6-O-(4-chlorobenzoyl)-α-D-alucopyranoside (47)

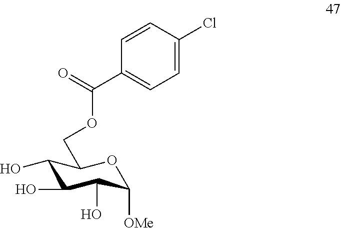

The product 47 (19.6 mg, 59%) was obtained as a white solid.

¹H NMR (400 MHz, Acetone-d₆) δ 8.13-7.93 (m, 2H), 7.69-7.44 (m, 2H), 4.72-4.56 (m, 2H), 4.46 (dd, J=11.7, 6.1 Hz, 1H), 3.87 (ddd, J=10.1, 6.2, 2.2 Hz, 1H), 3.69 (t, J=9.1 Hz, 1H), 3.50-3.41 (m, 2H), 3.39 (s, 3H). ¹³C NMR (101 MHz, Acetone-d₆) δ 164.98, 138.79, 131.07, 129.13, 128.82, 100.08, 74.27, 72.53, 70.75, 69.77, 64.50, 54.45. ESI-MS: calcd for C₁₄H₁₇O₇CINa [M+Na]⁺: 355.0561, found: 355.0547.

### Methyl-6-O-(4-chlorobenzoyl)-β-D-glucopyranoside (48)

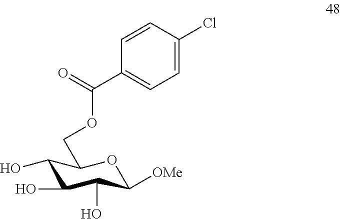

The product 48 (19.9 mg, 60%) was obtained as a white solid.

¹H NMR (500 MHz, DMSO-d₆) δ 8.07-7.78 (m, 2H), 7.75-7.49 (m, 2H), 5.27 (d, J=4.6 Hz, 1H), 5.15 (d, J=4.9 Hz, 1H), 5.09 (s, 1H), 4.56 (dd, J=11.7, 2.1 Hz, 1H), 4.35 (dd, J=11.8, 6.1 Hz, 1H), 4.13 (d, J=7.8 Hz, 1H), 3.57-3.44 (m, 1H), 3.36 (s, 3H), 3.22 (d, J=5.1 Hz, 2H), 3.01 (q, J=7.9, 7.3 Hz, 1H). ¹³C NMR (101 MHz, DMSO-d₆) δ 165.28, 138.76, 131.46, 129.48, 129.07, 104.39, 76.79, 74.00, 73.77, 70.49, 64.92, 56.35. ESI-MS: calcd for C₁₄H₁₇O₇CINa [M+Na]⁺: 355.0561, found: 355.0559.

### Phenyl-6-O-(4-chlorobenzoyl)-β-D-alucopyranoside (49)

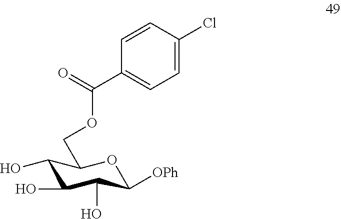

The product 49 (27.6 mg, 70%) was obtained as a white solid.

¹H NMR (400 MHz, Acetone-d₆) δ 8.10-7.99 (m, 2H), 7.69-7.54 (m, 2H), 7.27-7.20 (m, 2H), 7.09 (d, J=7.9 Hz, 2H), 6.99 (t, J=7.3 Hz, 1H), 5.05 (d, J=7.4 Hz, 1H), 4.76 (dd, J=11.8, 2.2 Hz, 2H), 4.65 (s, 1H), 4.58 (s, 1H), 4.44 (dd, J=11.8, 7.3 Hz, 1H), 3.95 (ddd, J=9.5, 7.3, 2.2 Hz, 1H), 3.67-3.47 (m, 3H). ¹³C NMR (101 MHz, Acetone-d₆) δ 164.85, 157.78, 138.84, 131.12, 129.22, 129.07, 128.79, 122.00, 116.42, 100.80, 76.98, 74.02, 73.77, 70.60, 64.50. ESIMS: calcd for C₁₉H₁₉O₇CINa [M+Na]⁺: 417.0717, found: 417.0711.

### Octyl-6-O-(4-chlorobenzoyl)-β-D-glucopyranoside (50)

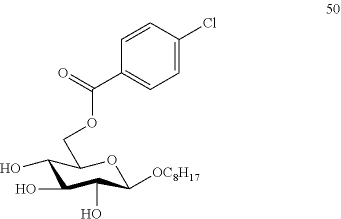

The product 50 (26.7 mg, 62%) was obtained as a white solid.

¹H NMR (400 MHz, Acetone-d₆) δ 8.11-7.86 (m, 2H), 7.72-7.19 (m, 2H), 4.67 (dd, J=11.7, 2.2 Hz, 1H), 4.47 (dd, J=11.7, 6.2 Hz, 2H), 4.33 (d, J=7.7 Hz, 2H), 4.27 (s, 1H), 3.78 (dt, J=9.8, 6.7 Hz, 1H), 3.65 (dq, J=6.2, 3.3, 2.2 Hz, 1H), 3.55-3.49 (m, 1H), 3.49-3.41 (m, 2H), 3.23 (dd, J=9.5, 6.3 Hz, 1H), 1.55 (p, J=6.9 Hz, 2H), 1.37-1.16 (m, 1OH), 0.86 (dt, J=13.3, 6.9 Hz, 3H). ¹³C NMR (101 MHz, Acetone-d₆) δ 166.16, 139.76, 131.21, 128.77, 128.16, 102.69, 76.12, 73.91, 73.59, 70.45, 70.27, 64.23, 31.82, 29.62, 29.37, 29.27, 25.90, 22.65, 14.09. ESI-MS: calcd for C₂₁H₃₁O₇ CINa [M+Na]⁺: 453.1656, found: 453.1654.

### Methyl-6-O-(4-chlorobenzoyl)-β-D-galactopyranoside (51)

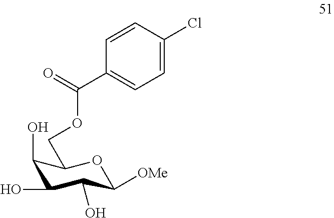

The product 51 (20.9 mg, 63%) was obtained as a white solid.

¹H NMR (400 MHz, Acetone-d₆) δ 8.18-7.92 (m, 2H), 7.63-7.45 (m, 2H), 4.54 (qd, J=11.1, 6.3 Hz, 2H), 4.19 (d, J=7.3 Hz, 1H), 4.00-3.91 (m, 2H), 3.62-3.49 (m, 2H), 3.44 (s, 3H). ¹³C NMR (101 MHz, Acetone-d₆) δ 164.90, 138.82, 131.09, 129.08, 128.80, 104.57, 73.54, 72.35, 71.23, 68.78, 64.13, 55.68. ESI-MS: calcd for C₁₄H₁₇O₇CINa [M+Na]⁺: 355.0561, found: 355.0552.

### Phenyl-6-O-(4-chlorobenzoyl)-β-D-galactopyranoside (52)

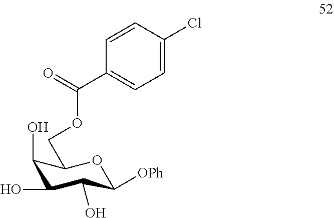

The product 52 (29.6 mg, 75%) was obtained as a white solid.

¹H NMR (400 MHz, Acetone-d₆) δ 8.11-7.98 (m, 2H), 7.68-7.51 (m, 2H), 7.23 (t, J=7.9 Hz, 2H), 7.09 (d, J=8.1 Hz, 2H), 6.98 (t, J=7.3 Hz, 1H), 4.99 (d, J=7.7 Hz, 1H), 4.59 (qd, J=11.4, 6.2 Hz, 3H), 4.31-4.20 (m, 2H), 4.08-4.04 (m, 2H), 3.91-3.85 (m, 1H), 3.76 (dd, J=9.5, 3.3 Hz, 1H). ¹³C NMR (101 MHz, Acetone-d₆) δ 164.85, 157.89, 138.88, 131.11, 129.19, 129.04, 128.80, 121.91, 116.45, 101.18, 73.57, 72.78, 71.05, 68.87, 64.46. ESI-MS: calcd for C₁₉H₁₉O₇CINa [M+Na]⁺: 417.0717, found: 417.0696.

### (4-methoxyphenyl)-6-O-(4-chlorobenzoyl)-β-D-galactopyranoside (53)

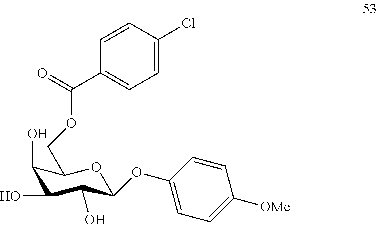

The product 53 (28.8 mg, 68%) was obtained as a white solid.

¹H NMR (400 MHz, DMSO-d₆) δ 8.06-7.83 (m, 2H), 7.65 (d, J=8.5 Hz, 2H), 6.98-6.83 (m, 2H), 6.78-6.51 (m, 2H), 5.23 (d, J=5.0 Hz, 1H), 4.98 (d, J=5.6 Hz, 1H), 4.86 (d, J=4.9 Hz, 1H), 4.73 (d, J=7.6 Hz, 1H), 4.51 (dd, J=11.3, 8.7 Hz, 1H), 4.34 (dd, J=11.3, 3.7 Hz, 1H), 4.00 (dd, J=9.0, 3.7 Hz, 1H), 3.77 (t, J=4.2 Hz, 1H), 3.66 (s, 3H), 3.57 (td, J=8.6, 7.8, 4.8 Hz, 1H), 3.50-3.42 (m, 1H). ¹³C NMR (101 MHz, DMSO-d₆) δ 165.11, 154.58, 151.69, 138.82, 131.53, 129.43, 128.99, 117.85, 114.62, 101.99, 73.49, 72.83, 70.60, 68.82, 64.97, 55.66. ESI-MS: calcd for C₂₀H₂₁O₈ CINa [M+Na]⁺: 447.0823, found: 447.0817.

### 4-Nitrophenyl-6-O-(4-chlorobenzoyl)-α-D-galactopyranoside (54)

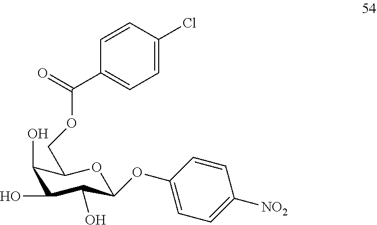

The product 54 (30.3 mg, 69%) was obtained as a white solid.

¹H NMR (400 MHz, Acetone-d₆) δ 8.09 (dd, J=13.7, 8.9 Hz, 4H), 7.71-7.39 (m, 2H), 7.31-7.08 (m, 2H), 5.22 (d, J=7.7 Hz, 1H), 4.80 (s, 1H), 4.66 (dd, J=11.5, 8.4 Hz, 1H), 4.56 (dd, J=11.5, 4.0 Hz, 1H), 4.41 (s, 1H), 4.32 (ddd, J=8.4, 4.1, 1.2 Hz, 1H), 4.20-4.14 (m, 1H), 4.10 (d, J=3.5 Hz, 1H), 3.94 (dd, J=9.5, 7.7 Hz, 1H), 3.79 (dd, J=9.5, 3.4 Hz, 1H). ¹³C NMR (101 MHz, Acetone-d₆) δ 164.78, 162.49, 142.31, 139.02, 131.13, 128.95, 128.84, 125.36, 116.51, 100.49, 73.42, 73.13, 70.76, 68.72, 64.23. ESI-MS: calcd for C₁₉H₁₃O₉CINNa [M+Na]⁺: 462.0568, found: 462.0554.

### Isopropylthio-6-O-(4-chlorobenzoyl)-β-D-galactopyranoside (55)

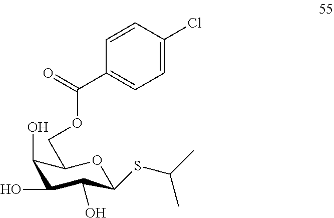

The product 55 (25.6 mg, 68%) was obtained as a white solid.

¹H NMR (400 MHz, Acetone-d₆) δ 8.14-7.90 (m, 2H), 7.66-7.44 (m, 2H), 4.67-4.41 (m, 3H), 4.17-3.82 (m, 5H), 3.69-3.52 (m, 2H), 3.18 (p, J=6.8 Hz, 1H), 1.26 (d, J=6.7 Hz, 6H). ¹³C NMR (101 MHz, Acetone-d₆) δ 164.86, 138.82, 131.07, 129.07, 128.75, 85.46, 76.03, 74.88, 70.52, 69.15, 64.60, 34.41, 23.60, 23.29. ESI-MS: calcd for C₁₆H₂₁O₆CISNa [M+Na]⁺: 399.0645, found: 399.0641.

### Methyl 2-(benzyloxy)carbonyl)amino-2-deoxy-6-O-(4-chlorobenzoyl)-α-D-glucopyranoside (56)

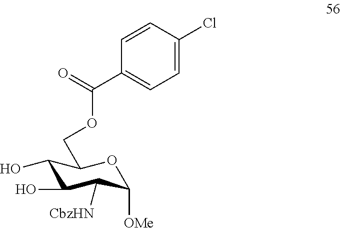

The product 56 (26.0 mg, 56%) was obtained as a white solid.

¹H NMR (400 MHz, Chloroform-d) δ 8.00 (d, J=8.6 Hz, 2H), 7.43 (d, J=8.6 Hz, 2H), 7.37 (m, 5H), 5.30 (d, J=9.3 Hz, 1H), 5.12 (q, J=12.0 Hz, 2H), 4.73 (d, J=3.7 Hz, 1H), 4.69-4.49 (m, 2H), 3.86 (m, 2H), 3.72 (t, J=9.5 Hz, 1H), 3.55 (t, J=9.3 Hz, 1H), 3.37 (s, 3H). ¹³C NMR (101 MHz, Chloroform-d) δ 166.08, 157.15, 139.75, 135.94, 131.15, 128.83, 128.60, 128.36, 128.31, 128.18, 98.66, 73.71, 71.01, 69.78, 67.42, 63.94, 55.26, 55.14. ESI-MS: calcd for C₂₂H₂₄NO₈ CINa [M+Na]⁺: 488.1088, found: 488.1071.

### 1,2-O-Isopropylidene-6-O-(4-chlorobenzoyl)-D-alucofuranose (58)

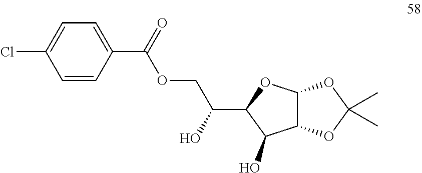 [SMILES: CC1(C)O[C@H]2O[C@H]([C@H](O)COC(=O)c3ccc(Cl)cc3)[C@H](O)[C@H]2O1]

The product 58 (24.3 mg, 68%) was obtained as a white solid.

¹H NMR (400 MHz, Acetone-d₆) δ 8.15-7.92 (m, 2H), 7.70-7.37 (m, 2H), 5.90 (d, J=3.7 Hz, 1H), 4.61 (dd, J=11.2, 2.4 Hz, 1H), 4.52 (dd, J=13.1, 4.1 Hz, 3H), 4.37 (dd, J=11.2, 6.2 Hz, 1H), 4.31 (tq, J=6.0, 3.3 Hz, 2H), 4.18 (dd, J=8.3, 2.8 Hz, 1H), 1.43 (s, 3H), 1.28 (s, 3H). ¹³C NMR (101 MHz, Acetone-d₆) δ 165.09, 138.71, 131.15, 129.26, 128.69, 111.04, 105.13, 85.32, 80.26, 74.21, 67.64, 66.73, 26.29, 25.62. ESI-MS: calcd for C₁₆H₁₇O₇ CINa [M+Na]⁺: 381.0717, found: 381.0708.

### Methyl-6-O-(4-chlorobenzoyl)-α-D-mannopyranoside (59)

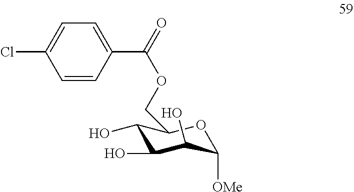

The product 59 (32.2 mg, 97%) was obtained as a white solid.

¹H NMR (400 MHz, Acetone-d₆) δ 8.16-7.87 (m, 2H), 7.61-7.41 (m, 2H), 4.67 (td, J=5.9, 5.0, 1.8 Hz, 2H), 4.47 (dd, J=11.7, 5.9 Hz, 1H), 4.30 (d, J=4.1 Hz, 1H), 4.01 (dd, J=11.1, 5.1 Hz, 2H), 3.89-3.73 (m, 3H), 3.72-3.64 (m, 1H), 3.37 (s, 3H). ¹³C NMR (101 MHz, Acetone-d₆) δ 165.04, 138.75, 131.08, 129.20, 128.78, 101.34, 71.69, 70.71, 70.62, 67.68, 64.70, 53.97. ESI-MS: calcd for C₁₄H₁₇O₇CINa [M+Na]⁺: 355.0561, found: 355.0565. 6-O-(4-chlorobenzoyl)-D-glucal (60)

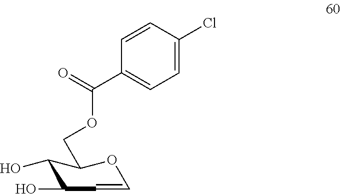

The product 60 (20.2 mg, 71%) was obtained.

¹H NMR (400 MHz, Acetone-d₆) δ 8.13-7.93 (m, 2H), 7.73-7.47 (m, 2H), 6.33 (dd, J=6.0, 1.7 Hz, 1H), 4.74 (dd, J=6.1, 2.2 Hz, 1H), 4.70 (dd, J=12.1, 2.3 Hz, 1H), 4.61 (dd, J=12.1, 5.3 Hz, 1H), 4.22 (dt, J=7.0, 2.0 Hz, 1H), 4.09 (ddd, J=9.8, 5.3, 2.3 Hz, 1H), 3.76 (dd, J=9.7, 7.0 Hz, 1H). ¹³C NMR (101 MHz, Acetone-d₆) δ 164.95, 142.96, 138.85, 131.12, 129.04, 128.81, 104.49, 76.40, 69.75, 69.11, 63.77. ESI-MS: calcd for C₁₃H₁₃O₅CINa [M+Na]⁺: 307.0349, found: 307.0334.

### Methyl-3-O-acetyl-α-D-glucopyranoside (61)

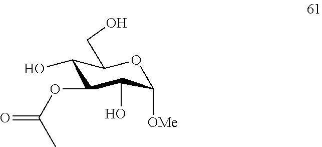

The product 61 (15.4 mg, 65%) was obtained as a white solid.

¹H NMR (400 MHz, Acetone-d₆) δ 5.13 (dd, J=9.9, 8.8 Hz, 1H), 4.71 (d, J=3.6 Hz, 1H), 4.43 (s, 1H), 3.81 (dd, J=11.4, 2.6 Hz, 1H), 3.71 (dd, J=12.0, 4.6 Hz, 3H), 3.63-3.45 (m, 3H), 3.40 (s, 3H), 2.03 (s, 3H). ¹³C NMR (101 MHz, Acetone-d₆) δ 170.48, 99.88, 76.09, 72.37, 70.72, 68.67, 61.48, 54.45, 20.28. ESI-MS: calcd for C₉H₁₇O₇[M+H]⁺: 237.0974, found: 237.0983.

### Methyl-3-O-(bis(2-chloroethyl)amino)phenyl)butanoyl)-α-D-glucopyranoside (63)

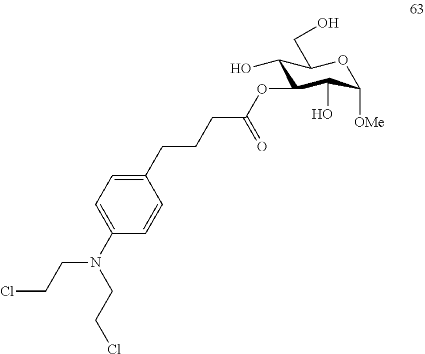

The product 63 (32.2 mg, 67%) was obtained as a white solid.

¹H NMR (400 MHz, Acetone-d₆) δ 7.18-6.99 (m, 2H), 6.85-6.54 (m, 2H), 5.18 (t, J=9.4 Hz, 1H), 4.71 (d, J=3.6 Hz, 1H), 4.40 (d, J=5.6 Hz, 1H), 3.85-3.67 (m, 1OH), 3.65-3.46 (m, 5H), 3.41 (s, 3H), 2.58 (t, J=7.6 Hz, 2H), 2.35 (t, J=7.4 Hz, 2H), 1.88 (p, J=7.5 Hz, 2H). ¹³C NMR (101 MHz, Acetone-d₆) δ 173.00, 144.72, 130.52, 129.57, 112.22, 99.95, 75.89, 72.47, 70.84, 68.76, 61.54, 54.45, 53.03, 40.76, 33.62, 33.38, 27.05. ESI-MS: calcd for C₂₁H₃₂O₇NCl₁₂ [M+H]⁺: 480.1556, found: 480.1567.

### Methyl-3-O-(2-(6-methoxynaphthyl) propionyl)-α-D-glucopyranoside (64)

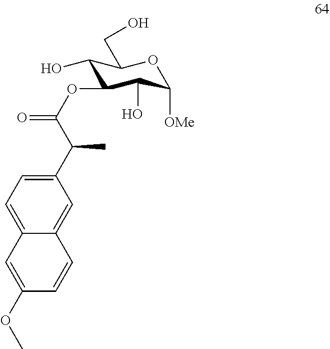

The product 64 (25.6 mg, 63%, dr 1:4) was obtained as a white solid.

¹H NMR (400 MHz, Chloroform-d) δ 7.72 (dd, J=8.9, 2.1 Hz, 3H), 7.43 (dd, J=8.6, 1.9 Hz, 1H), 7.19-7.01 (m, 2H), 5.07 (t, J=9.2 Hz, 1H), 4.73 (d, J=3.8 Hz, 1H), 3.99 (q, J=7.2 Hz, 1H), 3.93 (s, 3H), 3.88-3.79 (m, 2H), 3.67-3.58 (m, 2H), 3.55-3.49 (m, 1H), 3.42 (s, 3H), 1.63 (d, J=7.3 Hz, 3H). ¹³C NMR (101 MHz, Acetone-d₆) δ 174.14, 157.69, 136.50, 133.74, 129.20, 129.00, 126.77, 126.65, 125.92, 118.60, 105.55, 99.96, 76.38, 72.44, 70.83, 68.68, 61.51, 54.71, 54.45, 45.37, 18.76. ESI-MS: calcd for C₂₁H₂₆O₈Na [M+Na]⁺: 429.1525, found: 429.1524.

### Methyl-3-O-artesunate-α-D-glucopyranoside (65)

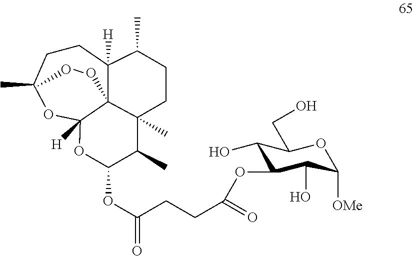

The product 65 (39.2 mg, 70%) was obtained as a white solid.

¹H NMR (400 MHz, Chloroform-d) δ 5.79 (d, J=9.9 Hz, 1H), 5.45 (s, 1H), 5.23-5.06 (m, 1H), 4.79 (d, J=3.8 Hz, 1H), 3.88 (d, J=3.3 Hz, 2H), 3.77-3.66 (m, 2H), 3.65-3.55 (m, 1H), 3.46 (s, 3H), 2.98-2.64 (m, 4H), 2.58 (ddd, J=9.9, 7.2, 4.5 Hz, 1H), 2.46-2.33 (m, 1H), 2.05 (ddd, J=14.8, 4.8, 3.0 Hz, 1H), 1.92 (ddd, J=13.8, 6.5, 3.4 Hz, 1H), 1.76 (ddt, J=16.4, 13.1, 3.7 Hz, 2H), 1.64 (dt, J=14.0, 4.4 Hz, 1H), 1.55-1.29 (m, 7H), 1.09-1.00 (m, 1H), 0.98 (d, J=5.9 Hz, 3H), 0.87 (d, J=7.1 Hz, 3H). ¹³C NMR (101 MHz, Chloroform-d) δ 172.68, 172.43, 104.65, 99.46, 92.49, 91.57, 80.13, 77.25, 71.15, 70.63, 69.07, 62.27, 55.39, 51.53, 45.18, 37.22, 36.17, 34.03, 31.60, 29.58, 29.42, 25.88, 24.56, 21.96, 20.18, 12.04. ESI-MS: calcd for C₂₆H₄₀O₁₃Na [M+Na]⁺: 583.2367, found: 583.2365.

### Methyl-3-O-dehydrocholyl-α-D-glucopyranoside (66)

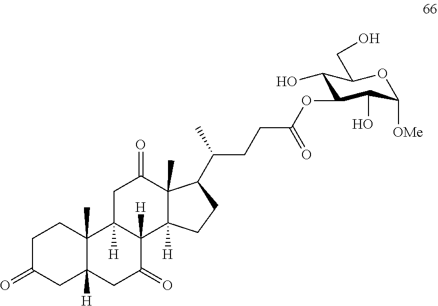

The product 66 (44.5 mg, 77%) was obtained as a white solid.

¹H NMR (400 MHz, Chloroform-d) δ 5.08 (t, J=9.1 Hz, 1H), 4.81 (d, J=3.8 Hz, 1H), 3.98-3.81 (m, 2H), 3.75-3.57 (m, 3H), 3.47 (s, 3H), 3.04-2.78 (m, 3H), 2.53 (ddd, J=14.9, 9.1, 5.3 Hz, 1H), 2.46-2.12 (m, 1OH), 2.09-1.98 (m, 4H), 1.97-1.80 (m, 2H), 1.64 (td, J=14.2, 13.4, 4.9 Hz, 3H), 1.48 (ddt, J=13.2, 8.4, 4.4 Hz, 1H), 1.42 (s, 3H), 1.38-1.24 (m, 3H), 1.09 (s, 3H), 0.88 (d, J=6.6 Hz, 3H). ¹³C NMR (101 MHz, Chloroform-d) δ 212.14, 209.11, 208.71, 175.92, 99.36, 77.22, 76.78, 71.41, 70.78, 69.39, 62.19, 56.93, 55.47, 51.78, 49.00, 46.85, 45.55, 44.98, 42.80, 38.63, 36.49, 36.02, 35.36, 35.29, 31.55, 30.40, 27.60, 25.14, 21.91, 18.64, 11.85. ESI-MS: calcd for C₃₁H₄₆O₁₀Na [M+Na]⁺: 601.2989, found: 601.2977.

### Methyl-3-O-(2-phenyl propionyl)-α-D-glucopyranoside (67)

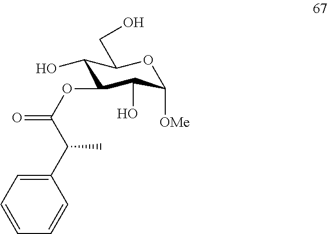

The product 67 (24.1 mg, 74%, dr 15:1) was obtained as a white solid.

¹H NMR (400 MHz, Chloroform-d) δ 7.39-7.27 (m, 5H), 5.02 (t, J=9.5 Hz, 1H), 4.76 (d, J=3.8 Hz, 1H), 3.89-3.66 (m, 3H), 3.63-3.46 (m, 3H), 3.42 (s, 3H), 2.25 (s, 1H), 2.17 (s, 1H), 1.98 (s, 1H), 1.51 (d, J=7.1 Hz, 3H). ¹³C NMR (101 MHz, Acetone-d₆) δ 174.04, 141.36, 128.29, 127.65, 126.63, 99.92, 76.33, 72.51, 70.81, 68.73, 61.45, 54.45, 45.47, 18.88. ESI-MS: calcd for C₁₆H₂₂O₇Na [M+Na]⁺: 349.1263, found: 349.1251.

### Methyl-3-O-(2-(4-isobutylphenyl) propionyl)-α-D-glucopyranoside (68)

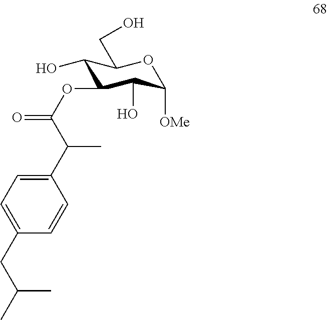

The product 68 (30.9 mg, 81%, dr 1.5:1) was obtained as a white solid.

¹H NMR (400 MHz, Chloroform-d) δ 7.22 (dd, J=8.2, 2.9 Hz, 2H), 7.11 (dd, J=8.2, 2.3 Hz, 2H), 5.03 (t, J=9.5 Hz, 1H), 4.77 (d, J=3.8 Hz, 1H), 3.86-3.73 (m, 3H), 3.67-3.48 (m, 3H), 3.43 (s, 3H), 2.45 (d, J=7.2 Hz, 2H), 1.85 (dt, J=13.5, 6.7 Hz, 1H), 1.52 (d, J=7.1 Hz, 3H), 0.90 (d, J=6.6 Hz, 6H). ¹³C NMR (101 MHz, Chloroform-d) δ (176.40)176.12, 140.83(140.71), 137.93(137.47), 129.53(129.40), (127.15)127.07, 99.46(99.32), 77.18(71.38), 71.15, 70.80(70.72), (69.09)68.95, (62.00)61.90, 55.41(55.38), (45.32)45.27, (45.01)44.99, 30.15, (22.37)22.35, (18.32)18.27. ESI-MS: calcd for C₂₀H₃₀O₇Na [M+Na]⁺: 405.1889, found: 405.1896.

### Methyl-3-O-(2-(2-fluoro-[1,1′-biphenyl-4-yl) propionyl)-α-D-glucopyranoside (69)

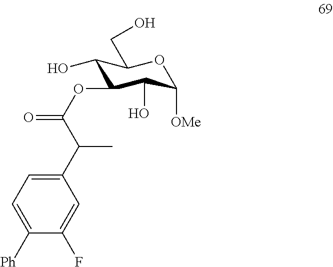

The product 69 (32.3 mg, 77%, dr 1.9:1) was obtained as a white solid.

¹H NMR (400 MHz, Chloroform-d) δ 7.55 (dt, J=8.1, 1.5 Hz, 2H), 7.50-7.34 (m, 4H), 7.22-6.94 (m, 2H), 5.11 (t, J=9.3 Hz, 1H), 4.79 (d, J=3.8 Hz, 1H), 4.05-3.74 (m, 3H), 3.72-3.55 (m, 3H), 3.46 (s, 3H), 2.84 (s, 1H), 2.49 (s, 1H), 2.28 (d, J=11.1 Hz, 1H), 1.58 (d, J=7.2 Hz, 3H). ¹³C NMR (101 MHz, Acetone-d₆) δ 173.51, 159.45 (d, J=245.9 Hz), (143.16 (d, J=8.0 Hz)143.14 (d, J=8.0 Hz), 135.57, 130.55 (d, J=3.7 Hz), 128.82 (d, J=3.0 Hz), 128.48, 127.13 (d, J=13.5 Hz)(127.11 (d, J=13.5 Hz), 127.06, 124.16 (d, J=3.2 Hz), 115.31 (d, J=23.8 Hz), (99.96)99.93, (96.63)76.54, 72.52(72.43), 70.80(70.69), 68.68(68.63), (61.48)61.43, (54.48)54.47, 44.96(44.91), 18.70(18.64). ESI-MS: calcd for C₂₂H₂₅O₇FNa [M+Na]⁺: 443.1482, found: 443.1476.

### Methyl-3-O-(2-(3-benzoylphenyl) propionyl)-α-D-glucopyranoside (70)

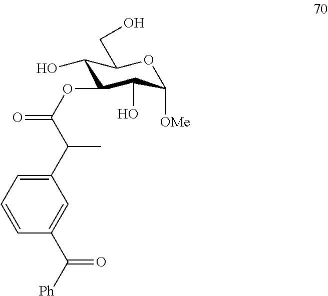

The product 70 (33.5 mg, 78%, dr 1.6:1) was obtained as a white solid.

¹H NMR (400 MHz, Chloroform-d) δ 7.90-7.77 (m, 3H), 7.68-7.54 (m, 3H), 7.47 (dtd, J=25.8, 7.9, 1.6 Hz, 3H), 5.12 (t, J=9.26 Hz, 1H), 4.78 (d, J=3.8 Hz, 1H), 3.93 (p, J=7.0 Hz, 1H), 3.84 (d, J=4.6 Hz, 2H), 3.70-3.52 (m, 3H), 3.45 (s, 3H), 3.26 (d, J=4.0 Hz, 1H), 3.14 (d, J=5.2 Hz, 1H), 2.40 (d, J=11.0 Hz, 1H), 1.58 (d, J=7.2 Hz, 3H). ¹³C NMR (101 MHz, Chloroform-d) δ 197.13(197.03), (175.31)175.20, 141.17(140.79), 137.99(137.86), (137.26)137.13, 132.81(132.71), (131.70)131.53, 130.26(130.22), (129.20)129.16, (129.12)129.04, 128.49(128.45), 128.38(128.36), 99.43, (77.19)77.08, 71.44, 70.82(70.76), (68.90)68.69, (61.98)61.87, 55.44, (45.68)45.46, (18.46)18.37. ESI-MS: calcd for C₂₃H₂₆O₈Na [M+Na]⁺: 453.1525, found: 453.1520.

### Methyl-3-O-(bis(2-chloroethyl)amino)phenyl)butanoyl)-β-D-alucopyranoside (71)

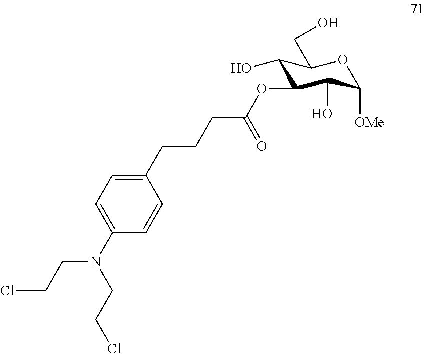

The product 71 (24.0 mg, 50%) was obtained as a white solid.

¹H NMR (400 MHz, Acetone-d₆) δ 7.26-6.98 (m, 2H), 6.85-6.58 (m, 2H), 5.00 (t, J=9.4 Hz, 1H), 4.30 (d, J=7.7 Hz, 1H), 3.86 (dd, J=11.7, 2.8 Hz, 1H), 3.82-3.68 (m, 1OH), 3.53 (t, J=9.5 Hz, 1H), 3.48 (s, 3H), 3.31 (dd, J=9.6, 7.8 Hz, 1H), 2.58 (t, J=7.6 Hz, 2H), 2.35 (t, J=7.5 Hz, 2H), 1.95-1.79 (m, 2H). ¹³C NMR (101 MHz, Acetone-d₆) δ 172.62, 144.73, 130.50, 129.57, 112.23, 104.13, 77.62, 76.48, 72.14, 68.96, 61.68, 55.98, 53.03, 40.76, 33.62, 33.35, 27.03. ESI-MS: calcd for C₂₁H₃₂O₇NCl₂ [M+H]⁺: 480.1556, found: 480.1548.

### 3-O-Dehydrocholyl-geniposide (72)

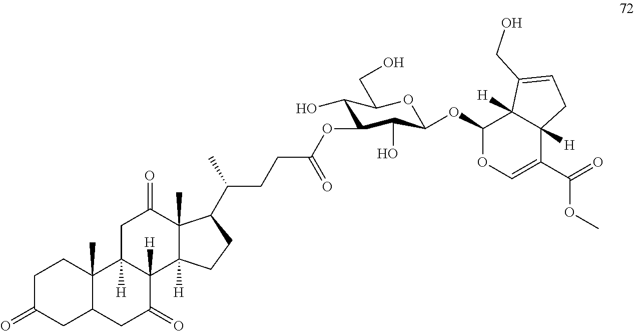

The product 72 (37.0 mg, 48%) was obtained as a white solid.

¹H NMR (400 MHz, Chloroform-d) δ 7.48 (d, J=1.3 Hz, 1H), 5.90 (s, 1H), 5.03-4.93 (m, 2H), 4.92-4.75 (m, 1H), 4.37-4.14 (m, 2H), 3.91-3.77 (m, 2H), 3.74 (s, 3H), 3.72-3.70 (m, 1H), 3.58-3.48 (m, 1H), 3.46-3.38 (m, 1H), 3.23 (q, J=8.2 Hz, 1H), 2.99-2.81 (m, 4H), 2.67 (t, J=8.0 Hz, 1H), 2.56-2.50 (m, 1H), 2.45-2.28 (m, 5H), 2.25 (s, 1H), 2.23-2.18 (m, 2H), 2.18-2.11 (m, 2H), 2.10-1.94 (m, 4H), 1.98-1.92 (m, 3H), 1.91-1.81 (m, 3H), 1.63 (td, J=14.5, 4.4 Hz, 1H), 1.54-1.43 (m, 1H), 1.41 (s, 3H), 1.38-1.23 (m, 3H), 1.08 (s, 3H), 0.87 (d, J=6.6 Hz, 3H). ¹³C NMR (101 MHz, Chloroform-d) δ 212.52, 209.34, 208.90, 175.46, 167.51, 151.44, 142.59, 130.00, 111.61, 100.20, 98.77, 77.51, 76.44, 71.96, 68.31, 61.16, 60.83, 56.95, 51.82, 51.40, 48.98, 46.84, 46.41, 45.53, 45.45, 44.98, 42.76, 38.94, 38.64, 36.46, 36.02, 35.64, 35.33, 35.26, 31.36, 30.34, 27.61, 25.12, 21.88, 18.65, 11.84. ESI-MS: calcd for C₄₁H₅₆O₁₄Na [M+Na]⁺: 795.3568, found: 795.3568.

### Methyl-3-O-(4-isopropylcyclohexane-1-carbonyl-D-phenylalanyl)-α-D-glucopyranoside (73)

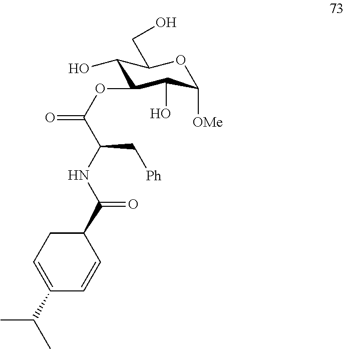

The product 73 (40.9 mg, 83%) was obtained as a white solid.

¹H NMR (400 MHz, Acetone-d₆) δ 7.53-7.13 (m, 5H), 5.21 (dd, J=10.0, 8.8 Hz, 1H), 4.72 (d, J=3.7 Hz, 1H), 4.62-4.39 (m, 1H), 3.81 (d, J=11.5 Hz, 1H), 3.76-3.66 (m, 1H), 3.65-3.49 (m, 3H), 3.41 (s, 3H), 3.27 (dd, J=13.9, 5.1 Hz, 1H), 3.05 (dd, J=13.9, 9.0 Hz, 1H), 2.13 (tt, J=12.3, 3.5 Hz, 1H), 1.84-1.75 (m, 4H), 1.48-1.28 (m, 3H), 1.07-0.93 (m, 3H), 0.86 (d, J=6.8 Hz, 6H). ¹³C NMR (101 MHz, DMSO-d₆) δ 175.85, 171.65, 138.17, 129.71, 128.50, 126.74, 99.92, 77.04, 72.92, 70.24, 68.13, 60.89, 54.89, 53.44, 44.26, 43.27, 37.28, 32.78, 29.58, 29.50, 28.97, 28.91, 20.10. ESI-MS: calcd for C₂₆H₄₀O₈N [M+H]⁺: 494.2754, found: 494.2755.

### Methyl-3-O-((benzyloxy)carbonyl)-D-phenylalanyl)-α-D-glucopyranoside (74)

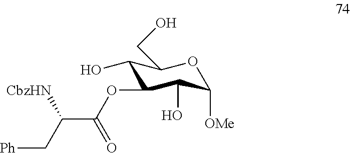

The product 74 (40.4 mg, 85%) was obtained as a white solid.

¹H NMR (400 MHz, Acetone-d₆) δ 7.46-7.02 (m, 10H), 6.65 (d, J=8.3 Hz, 1H), 5.22 (t, J=9.2 Hz, 1H), 5.05 (s, 2H), 4.73 (d, J=3.6 Hz, 1H), 4.53 (td, J=8.6, 4.8 Hz, 2H), 3.87-3.79 (m, 1H), 3.72 (dd, J=11.8, 4.6 Hz, 2H), 3.67-3.49 (m, 4H), 3.42 (s, 3H), 3.31 (dd, J=14.0, 4.8 Hz, 1H), 3.03 (dd, J=14.1, 9.0 Hz, 1H). ¹³C NMR (101 MHz, Acetone-d₆) δ 171.53, 156.23, 137.45, 137.16, 129.44, 128.33, 128.24, 127.78, 127.68, 126.51, 99.90, 77.65, 72.27, 70.57, 68.54, 65.92, 61.46, 55.68, 54.45, 37.29. ESI-MS: calcd for C₂₄H₂₉O₉NNa [M+Na]⁺: 498.1740, found: 498.1727.

### Methyl-3-O-(N-Cbz-L-leucine acyl)-α-D-glucopyranoside (75)

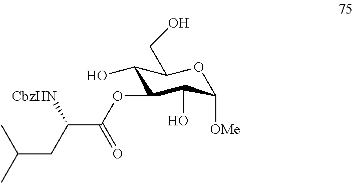

The product 75 (38.0 mg, 86%) was obtained as a white solid.

¹H NMR (400 MHz, Acetone-d₆) δ 7.52-7.06 (m, 5H), 6.73 (d, J=8.1 Hz, 1H), 5.15 (t, J=9.1 Hz, 1H), 5.08 (s, 2H), 4.68 (d, J=3.6 Hz, 1H), 4.41 (s, 1H), 4.28 (td, J=9.1, 8.6, 5.3 Hz, 1H), 3.80 (d, J=11.9 Hz, 1H), 3.69 (dd, J=12.8, 3.9 Hz, 1H), 3.62-3.50 (m, 4H), 3.46 (d, J=7.9 Hz, 1H), 3.39 (s, 3H), 1.81 (dq, J=12.7, 6.5 Hz, 1H), 1.67 (qdd, J=14.1, 9.0, 5.6 Hz, 2H), 0.93 (dd, J=6.6, 3.5 Hz, 6H). ¹³C NMR (101 MHz, Acetone-d₆) δ 173.57, 157.45, 138.09, 129.24, 128.72, 128.64, 100.79, 78.18, 73.18, 71.51, 69.47, 66.90, 62.40, 55.33, 53.81, 41.53, 25.38, 23.27, 21.89. ESI-MS: calcd for C₂₁H₃₂NO₉ [M+H]⁺: 442.2077, found: 442.2073.

### Methyl-3-O-(N-Cbz-L-methionine acyl)-α-D-glucopyranoside (76)

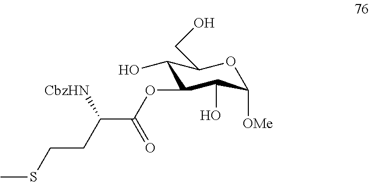

The product 76 (29.0 mg, 63%) was obtained as a white solid.

¹H NMR (400 MHz, Chloroform-d) δ 7.43-7.28 (m, 5H), 5.72 (d, J=7.1 Hz, 1H), 5.25-4.96 (m, 3H), 4.77 (d, J=3.8 Hz, 1H), 4.44 (q, J=7.1 Hz, 1H), 3.84 (d, J=3.0 Hz, 2H), 3.64 (d, J=8.4 Hz, 2H), 3.54 (d, J=9.3 Hz, 1H), 3.43 (s, 3H), 2.65-2.46 (m, 2H), 2.18 (tt, J=12.4, 5.8 Hz, 1H), 2.08-1.99 (m, 4H). ¹³C NMR (101 MHz, Chloroform-d) δ 172.72, 156.55, 135.92, 128.60, 128.35, 128.17, 99.40, 77.93, 71.15, 70.59, 68.59, 67.39, 61.99, 55.45, 53.66, 31.01, 29.81, 15.37. ESI-MS: calcd for C₂₀H₂₉NO₉SNa [M+Na]⁺: 482.1461, found: 482.1454.

### Methyl-3-O-(N-Cbz-L-valine acyl)-α-D-glucopyranoside (77)

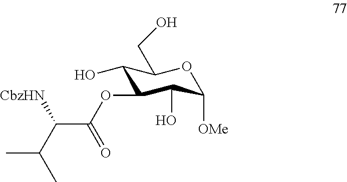

The product 77 (33.0 mg, 77%) was obtained as a white solid.

¹H NMR (400 MHz, Acetone-d₆) δ 7.53-7.17 (m, 5H), 6.58 (d, J=8.4 Hz, 1H), 5.18 (t, J=8.9 Hz, 1H), 5.08 (s, 2H), 4.69 (d, J=3.6 Hz, 1H), 4.48 (s, 1H), 4.19 (dd, J=8.5, 5.0 Hz, 1H), 3.80 (d, J=11.5 Hz, 1H), 3.70 (d, J=11.0 Hz, 1H), 3.60 (q, J=8.5, 6.9 Hz, 4H), 3.49 (td, J=9.2, 3.3 Hz, 1H), 3.39 (s, 3H), 2.23 (dq, J=13.3, 6.6 Hz, 1H), 0.99 (t, J=7.5 Hz, 6H). ¹³C NMR (101 MHz, Acetone-d₆) δ 171.67, 156.66, 137.19, 128.35, 127.82, 127.80, 99.93, 77.17, 72.31, 70.64, 68.55, 66.04, 61.47, 59.82, 54.46, 30.65, 18.53, 17.12. ESI-MS: calcd for C₂₀H₂₉NO₉Na [M+Na]⁺: 450.1740, found: 450.1738.

### Methyl-3-O-(N-Boc-O-benzyl-L-serine acyl)-α-D-glucopyranoside (78)

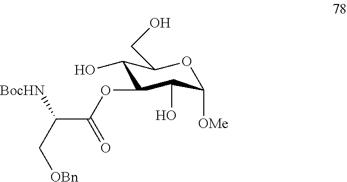

The product 78 (35.0 mg, 74%) was obtained as a white solid.

¹H NMR (400 MHz, Chloroform-d) δ 7.41-6.97 (m, 5H), 5.49 (d, J=7.6 Hz, 1H), 5.17 (t, J=8.7 Hz, 1H), 4.79 (d, J=3.7 Hz, 1H), 4.54 (s, 2H), 4.40 (s, 1H), 3.94-3.78 (m, 3H), 3.77-3.56 (m, 4H), 3.44 (s, 3H), 1.44 (s, 9H). ¹³C NMR (101 MHz, Chloroform-d) δ 171.17, 156.10, 137.14, 128.55, 128.05, 127.93, 99.34, 80.73, 78.38, 73.59, 71.13, 70.45, 69.55, 68.78, 62.04, 55.38, 54.42, 28.29. ESI-MS: calcd for C₂₂H₃₃NO₁₀Na [M+Na]⁺: 494.2002, found: 494.1996.

### Methyl-3-O-(N-Boc-O-benzyl-L-threonine acyl)-α-D-alucopyranoside (79)

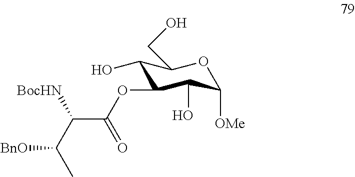

The product 79 (34.4 mg, 71%) was obtained as a white solid.

¹H NMR (400 MHz, Acetone-d₆) δ 7.43-7.03 (m, 5H), 5.94 (d, J=8.9 Hz, 1H), 5.37-5.11 (m, 1H), 4.71 (d, J=3.3 Hz, 1H), 4.57 (d, J=2.8 Hz, 2H), 4.28 (dd, J=8.9, 3.1 Hz, 1H), 4.19 (qd, J=6.2, 2.8 Hz, 1H), 3.81 (d, J=11.6 Hz, 1H), 3.71 (d, J=12.3 Hz, 1H), 3.67-3.52 (m, 3H), 3.39 (s, 3H), 1.42 (s, 9H), 1.25 (d, J=6.3 Hz, 3H). ¹³C NMR (101 MHz, Acetone-d₆) δ 170.88, 156.03, 138.90, 128.01, 127.81, 127.22, 99.88, 78.83, 77.66, 74.90, 72.33, 70.95, 70.75, 70.64, 68.57, 61.47, 58.39, 54.46, 27.63, 16.39. ESI-MS: calcd for C₂₃H₃₆NO₁₀ [M+H]⁺: 486.2339, found: 486.2335.

### Methyl-3-O-(Methyl L-α-aspartyl-L-phenylalaninate)-α-D-glucopyranoside (80)

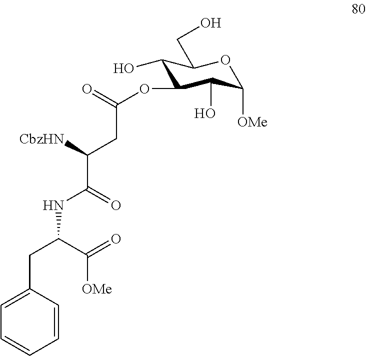

The product 80 (40.0 mg, 66%) was obtained as a white solid.

¹H NMR (400 MHz, Acetone-d₆) δ 7.68 (d, J=7.8 Hz, 1H), 7.46-7.12 (m, 10H), 6.75 (d, J=8.6 Hz, 1H), 5.16 (t, J=9.3 Hz, 1H), 5.08 (s, 2H), 4.74-4.56 (m, 3H), 4.38 (s, 1H), 3.83-3.72 (m, 2H), 3.67 (s, 4H), 3.62-3.48 (m, 4H), 3.38 (s, 3H), 3.18-2.99 (m, 2H), 2.75 (dd, J=15.8, 6.6 Hz, 2H). ¹³C NMR (101 MHz, Acetone-d₆) δ 171.35, 170.96, 170.04, 169.66, 136.95, 136.68, 129.28, 128.37, 127.86, 127.81, 126.73, 99.84, 77.11, 72.17, 70.49, 68.65, 66.32, 61.53, 54.39, 53.82, 51.58, 37.12, 36.73. ESI-MS: calcd for C₂₉H₃₇N₂O₁₂ [M+H]⁺: 605.2346, found: 605.2347.

### Methyl-3-O-(Na-Boc-N-in-Boc-L-tryophan acyl)-α-D-alucopyranoside (81)

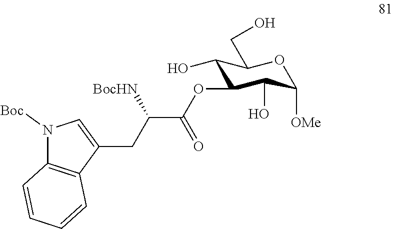

The product 81 (44 mg, 76%) was obtained as a white solid.

¹H NMR (400 MHz, Acetone-d₆) δ 8.13 (d, J=8.2 Hz, 1H), 7.64 (d, J=8.3 Hz, 2H), 7.36-7.29 (m, 1H), 7.28-7.21 (m, 1H), 6.40 (d, J=7.9 Hz, 1H), 5.25 (dd, J=9.8, 8.0 Hz, 1H), 4.73 (d, J=3.5 Hz, 1H), 4.51 (td, J=8.2, 4.9 Hz, 1H), 3.81 (d, J=11.5 Hz, 1H), 3.75-3.67 (m, 1H), 3.66-3.55 (m, 3H), 3.41 (s, 4H), 3.15 (dd, J=14.9, 8.5 Hz, 1H), 1.66 (s, 9H), 1.37 (s, 9H). ¹³C NMR (101 MHz, Acetone-d₆) δ 171.64, 155.76, 149.41, 135.44, 130.85, 124.47, 124.14, 122.39, 119.03, 116.27, 114.96, 99.93, 83.22, 78.89, 77.69, 72.23, 70.55, 68.59, 61.47, 54.48, 54.15, 27.65, 27.38, 26.95. ESI-MS: calcd for C₂₈H₄₁N₂O₁₁ [M+H]⁺: 581.2710, found: 581.2706.

### Methyl-3-O-(Na-Cbz-NE-Boc-L-Lysine acyl)-α-D-glucopyranoside (82)

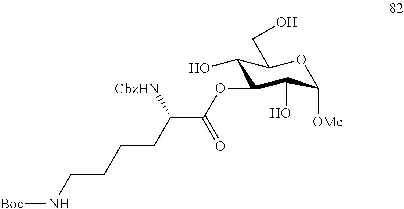

The product 82 (39.0 mg, 70%) was obtained as a white solid.

¹H NMR (400 MHz, Acetone-d₆) δ 7.65-7.14 (m, 5H), 6.74 (d, J=7.6 Hz, 1H), 5.98 (s, 1H), 5.18 (t, J=9.1 Hz, 1H), 5.10 (s, 2H), 4.70 (d, J=3.6 Hz, 1H), 4.25 (q, J=7.2, 6.8 Hz, 1H), 3.82 (dd, J=11.7, 2.2 Hz, 1H), 3.71 (dd, J=11.7, 4.4 Hz, 1H), 3.65-3.46 (m, 3H), 3.41 (s, 3H), 3.07 (d, J=6.0 Hz, 2H), 1.91 (q, J=6.8, 5.1 Hz, 1H), 1.79 (t, J=7.1 Hz, 1H), 1.50 (t, J=4.9 Hz, 4H), 1.41 (s, 9H). ¹³C NMR (101 MHz, Acetone-d₆) δ 172.20, 156.46, 155.96, 137.16, 128.35, 127.83, 127.81, 99.91, 77.59, 77.25, 72.32, 70.54, 68.57, 66.03, 61.48, 54.44, 39.79, 31.30, 27.79, 22.42. ESI-MS: calcd for C₂₆H₄₁N₂O₁₁ [M+H]⁺: 557.2710, found: 557.2708.

### (4-methoxyphenyl)-2-O-artesunate-β-D-galactopyranoside (83)

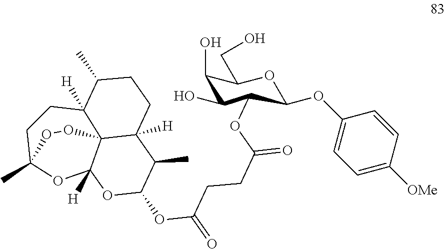

The product 83 (42.4 mg, 65%) was obtained as a white solid.

¹H NMR (400 MHz, Chloroform-d) δ 7.04-6.89 (m, 2H), 6.88-6.66 (m, 2H), 5.75 (d, J=9.8 Hz, 1H), 5.39 (s, 1H), 5.28 (dd, J=9.8, 8.0 Hz, 1H), 4.87 (d, J=8.0 Hz, 1H), 4.10 (d, J=3.4 Hz, 1H), 3.91 (qd, J=11.8, 5.6 Hz, 2H), 3.76 (s, 4H), 3.65 (t, J=5.7 Hz, 1H), 2.86-2.68 (m, 4H), 2.55 (ddd, J=9.8, 7.2, 4.5 Hz, 1H), 2.37 (td, J=14.0, 3.9 Hz, 1H), 2.03 (ddd, J=14.4, 4.9, 2.1 Hz, 1H), 1.89 (ddt, J=13.5, 6.5, 3.5 Hz, 1H), 1.73 (ddt, J=13.6, 10.1, 3.6 Hz, 2H), 1.61 (dt, J=13.9, 4.4 Hz, 1H), 1.43 (s, 4H), 1.38-1.22 (m, 3H), 1.08-0.93 (m, 4H), 0.83 (d, J=7.1 Hz, 3H). ¹³C NMR (101 MHz, Chloroform-d) δ 172.01, 171.34, 155.45, 151.29, 118.58, 114.57, 104.58, 100.53, 92.34, 91.55, 80.13, 74.58, 72.98, 72.11, 69.36, 61.93, 55.63, 51.53, 45.18, 37.19, 36.20, 34.06, 31.73, 29.33, 29.12, 25.88, 24.56, 21.93, 20.19, 12.03. ESI-MS: calcd for C₃₂H₄₄O₁₄Na [M+Na]⁺: 675.2629, found: 675.2628.

### (4-methoxyphenyl)-2-O-(4-(N,N-dipropylsulfamoyl)benzoyl)-β-D-galactopyranoside (84)

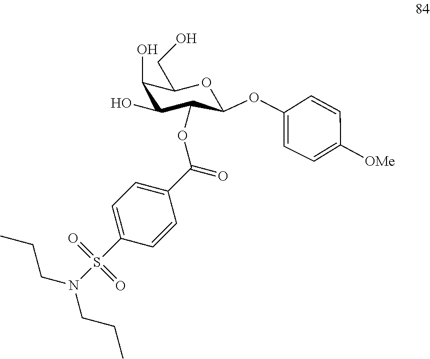

The product 84 (37.8 mg, 68%) was obtained as a white solid.

¹H NMR (400 MHz, Chloroform-d) δ 8.17 (d, J=8.3 Hz, 2H), 7.89 (d, J=8.4 Hz, 2H), 7.03-6.86 (m, 2H), 6.85-6.67 (m, 2H), 5.52 (dd, J=9.7, 8.0 Hz, 1H), 5.07 (d, J=8.0 Hz, 1H), 4.21 (d, J=3.4 Hz, 1H), 4.09-3.98 (m, 2H), 3.92 (dd, J=9.9, 3.3 Hz, 1H), 3.77 (m, 4H), 3.26-2.91 (m, 4H), 1.57 (h, J=7.4 Hz, 4H), 0.89 (t, J=7.4 Hz, 6H). ¹³C NMR (101 MHz, Chloroform-d) 5 165.49, 155.62, 151.03, 144.67, 132.83, 130.56, 127.07, 118.42, 114.63, 100.71, 74.47, 73.96, 72.34, 69.58, 61.95, 55.60, 50.02, 22.00, 11.15. ESI-MS: calcd for C₂H₃₅O₁₀NSNa [M+Na]⁺: 576.1879, found: 576.1880.

### Methyl-3-O-(3-phenylpropanoyl)-α-D-glucopyranoside (85)

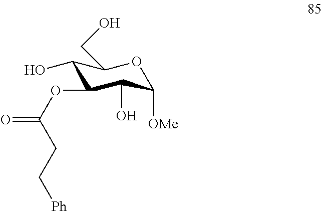

The product 85 (14.7 mg, 45%) was obtained as a white solid.

¹H NMR (400 MHz, Acetone-d₆) δ 7.28 (d, J=4.4 Hz, 4H), 7.19 (dt, J=8.8, 4.1 Hz, 1H), 5.18 (t, J=9.3 Hz, 1H), 4.71 (d, J=3.6 Hz, 1H), 4.36 (d, J=5.4 Hz, 1H), 3.85-3.77 (m, 1H), 3.72 (dt, J=11.6, 5.4 Hz, 1H), 3.66-3.48 (m, 5H), 3.41 (s, 3H), 2.94 (t, J=7.9 Hz, 2H), 2.74-2.58 (t, J=7.9 Hz, 2H). ¹³C NMR (101 MHz, Acetone-d₆) δ 172.41, 141.06, 128.34, 128.26, 125.98, 99.94, 76.24, 72.42, 70.79, 68.73, 61.51, 54.43, 35.66, 30.62. ESI-MS: calcd for C₁₆H₂₂O₇Na [M+Na]⁺: 349.1263, found: 349.1273.

### Methyl-α-D-glucopyranoside conjugated succinyl paclitaxel (95)

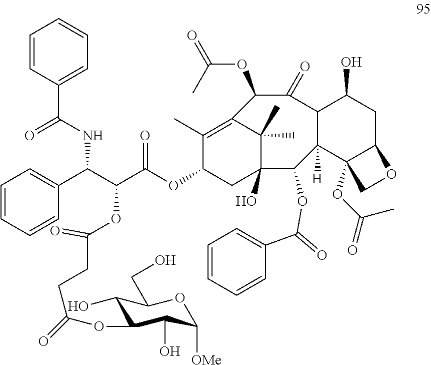

The product 95 (70 mg, 62%) was obtained as a white solid.

¹H NMR (400 MHz, Acetone-d₆) δ 8.46 (d, J=9.1 Hz, 1H), 8.24-8.12 (m, 2H), 7.98-7.83 (m, 2H), 7.75-7.67 (m, 1H), 7.66-7.59 (m, 4H), 7.57-7.41 (m, 5H), 7.33 (t, J=7.4 Hz, 1H), 6.43 (s, 1H), 6.17 (t, J=9.2 Hz, 1H), 6.04-5.94 (m, 1H), 5.70 (d, J=7.2 Hz, 1H), 5.59 (d, J=5.9 Hz, 1H), 5.16 (t, J=9.1 Hz, 1H), 4.99 (d, J=7.8 Hz, 1H), 4.69 (d, J=3.6 Hz, 1H), 4.44 (dt, J=11.2, 6.0 Hz, 1H), 4.35 (d, J=4.8 Hz, 1H), 4.25-4.13 (m, 2H), 3.89 (s, 1H), 3.86 (d, J=7.2 Hz, 1H), 3.83-3.76 (m, 1H), 3.74-3.66 (m, 1H), 3.63-3.46 (m, 6H), 3.40 (s, 3H), 2.78-2.72 (m, 2H), 2.71-2.65 (m, 2H), 2.53-2.45 (m, 4H), 2.36 (dd, J=15.4, 9.4 Hz, 1H), 2.18 (s, 3H), 1.96 (d, J=1.5 Hz, 3H), 1.80 (ddd, J=13.9, 11.0, 2.3 Hz, 1H), 1.68 (s, 3H), 1.24-1.10 (m, 7H). ¹³C NMR (101 MHz, Chloroform-d) δ 203.77, 172.81, 171.46, 171.23, 169.97, 168.49, 167.33, 167.01, 142.46, 136.90, 133.70, 133.66, 132.90, 131.83, 130.28, 129.25, 129.09, 128.73, 128.54, 127.41, 126.69, 99.28, 84.44, 81.13, 79.12, 75.57, 75.07, 74.85, 72.44, 72.07, 71.15, 70.69, 68.64, 61.93, 58.50, 55.38, 52.91, 45.64, 43.21, 35.60, 35.52, 29.67, 29.43, 26.77, 22.69, 22.14, 20.82, 14.80, 9.62. ESI-MS: calcd for C₅₈H₆₇NO₂₂Na [M+Na]⁺: 1152.4052, found: 1152.4066.

### Phenyl-α-D-galactoside conjugated succinyl paclitaxel (96)

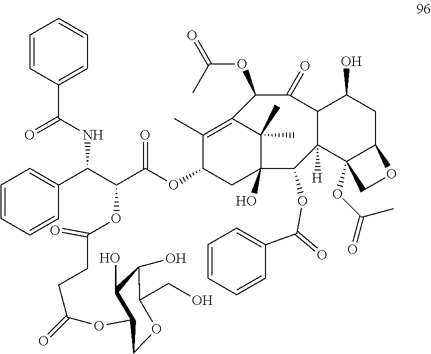

The product 96 (79.8 mg, 67%) was obtained as a white solid (axial chirality exist in this compound).

¹H NMR (400 MHz, Acetone-d₆) δ 8.49 (d, J=9.1 Hz, 1H), 8.20-8.11 (m, 2H), 7.93-7.84 (m, 2H), 7.74-7.65 (m, 1H), 7.61 (td, J=7.0, 6.5, 1.6 Hz, 4H), 7.56-7.40 (m, 5H), 7.35-7.20 (m, 3H), 7.10-6.92 (m, 3H), 6.42 (s, 1H), 6.18 (t, J=9.1 Hz, 1H), 5.99 (dd, J=9.1, 5.8 Hz, 1H), 5.70 (d, J=7.2 Hz, 1H), 5.55 (d, J=5.7 Hz, 1H), 5.33 (dd, J=9.9, 8.0 Hz, 1H), 5.03 (d, J=8.0 Hz, 1H), 5.01-4.96 (m, 1H), 4.43 (dt, J=11.4, 6.1 Hz, 1H), 4.28-4.12 (m, 4H), 4.09-3.98 (m, 2H), 3.92-3.88 (m, 1H), 3.87-3.76 (m, 5H), 3.56-3.45 (m, 1H), 2.79-2.61 (m, 4H), 2.53-2.45 (m, 4H), 2.36 (dd, J=15.4, 9.6 Hz, 1H), 2.17 (s, 3H), 1.97 (d, J=1.5 Hz, 3H), 1.79 (ddd, J=12.9, 11.0, 2.2 Hz, 1H), 1.68 (s, 3H), 1.20 (s, 3H), 1.19 (s, 3H). ¹³C NMR (101 MHz, Chloroform-d) δ 203.72, 171.99, 171.72, 171.26, 169.96, 168.20, 167.33, 166.96, 157.08, 142.49, 136.78, 133.71, 133.69, 132.80, 132.01, 130.25, 129.63, 129.25, 129.11, 129.04, 128.71, 127.21, 126.82, 123.13, 116.87, 99.21, 84.41, 81.10, 79.08, 75.62, 75.00, 74.61, 74.44, 73.23, 72.16, 72.04, 72.00, 69.59, 62.31, 58.45, 53.05, 45.74, 43.16, 35.64, 35.45, 29.00, 28.87, 26.70, 22.66, 22.00, 20.82, 14.84, 9.63. ESIMS: calcd for C₆₃H₆₉NO₂₂Na [M+Na]⁺: 1214.4209, found: 1214.4202.

Results and discussion

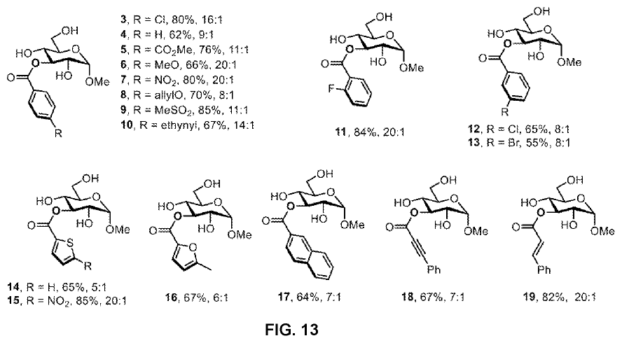

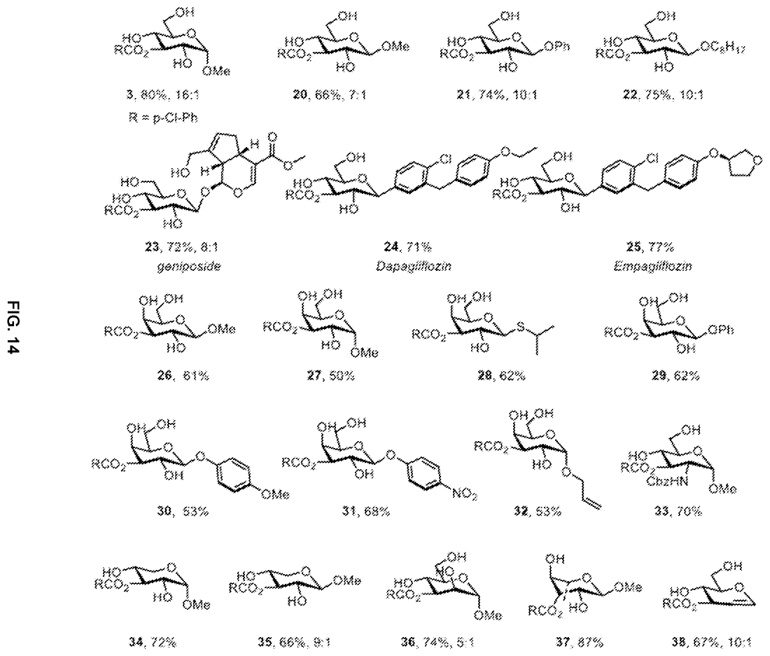

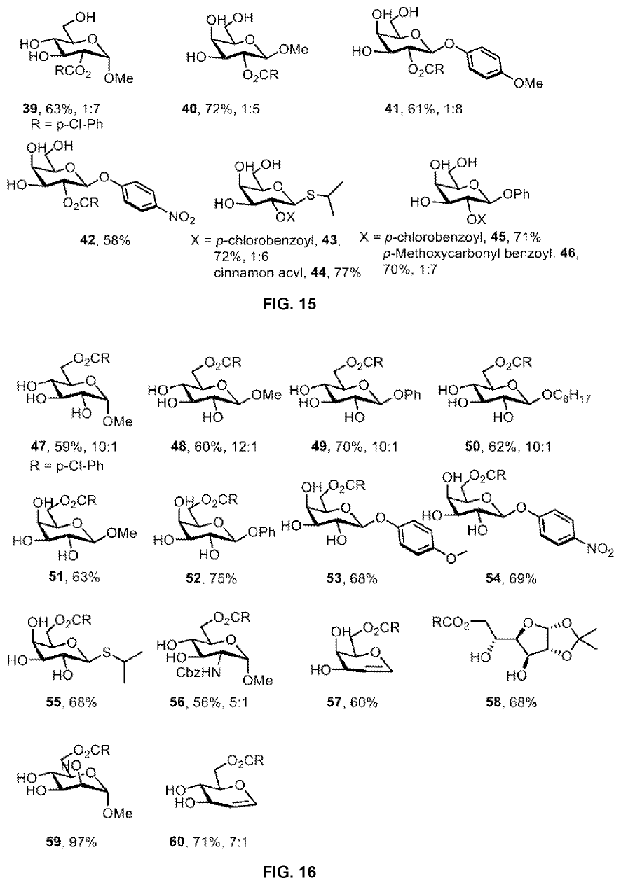

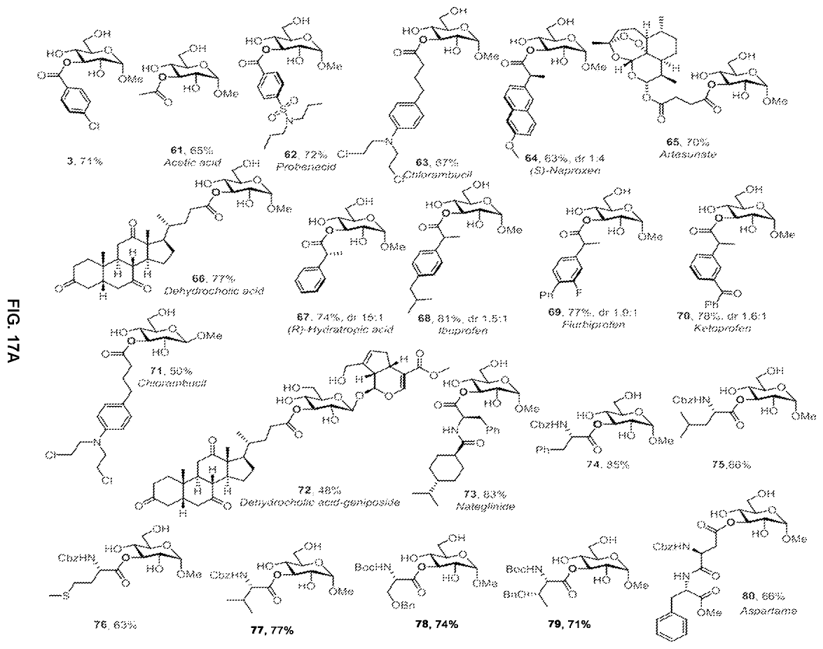

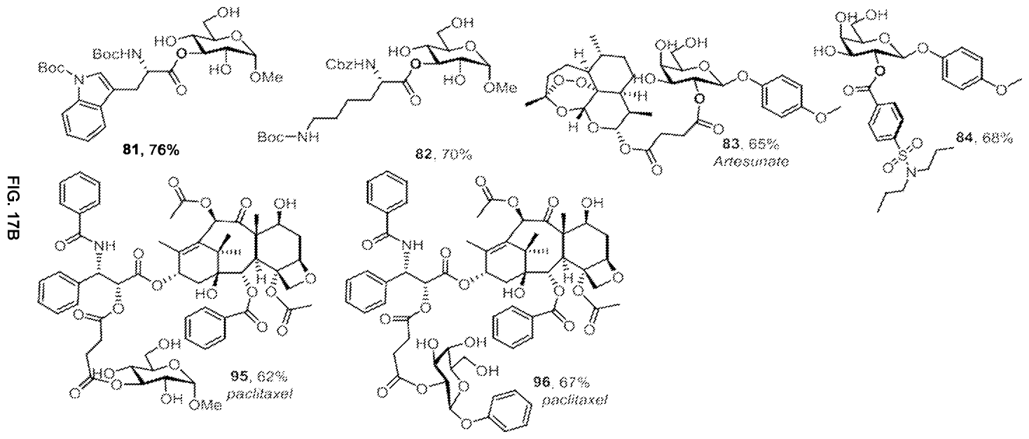

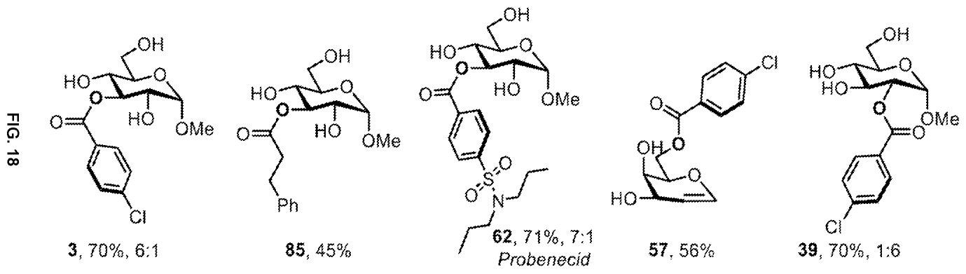

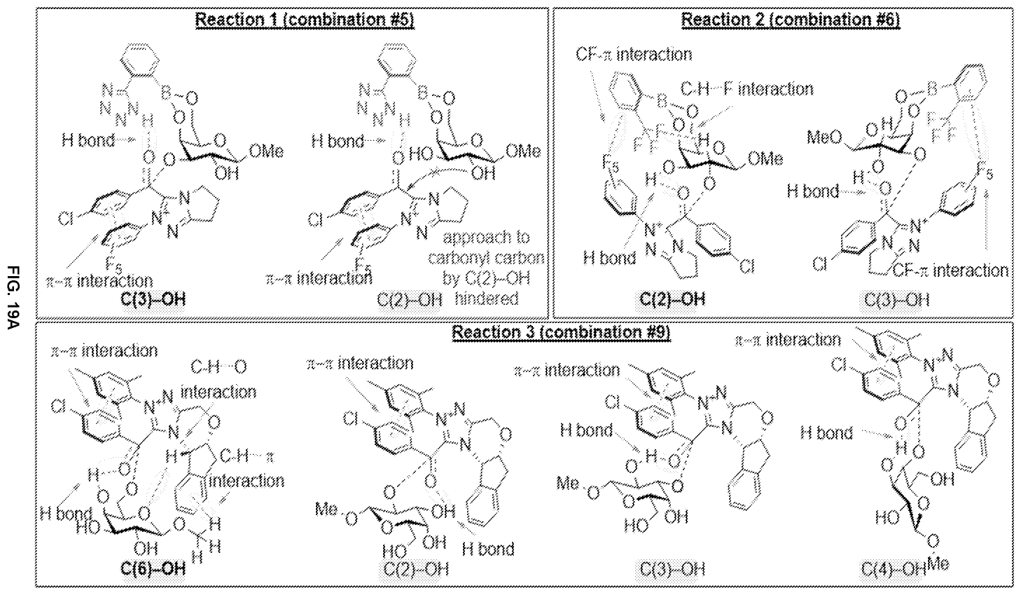

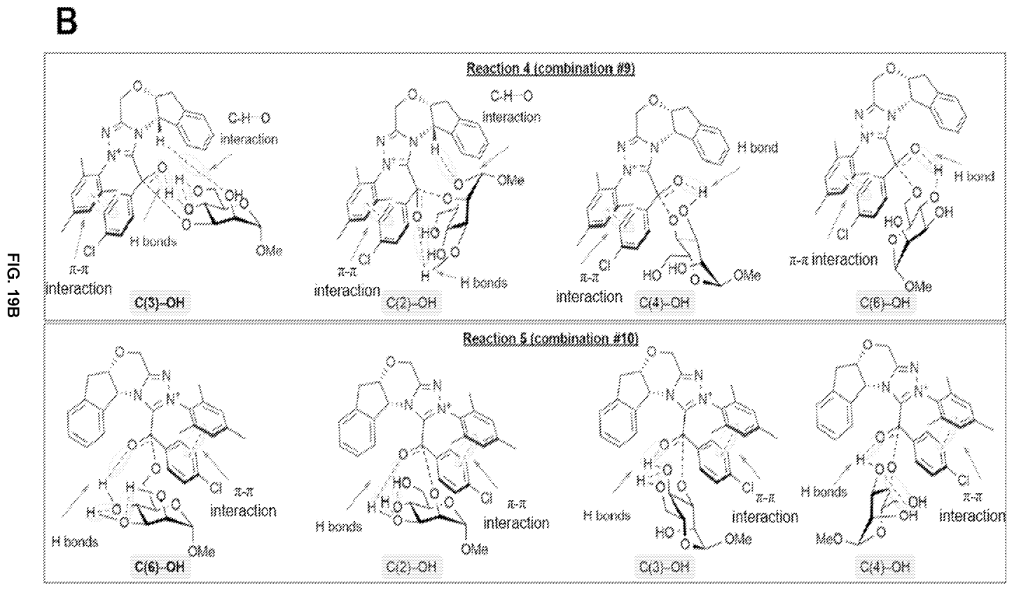

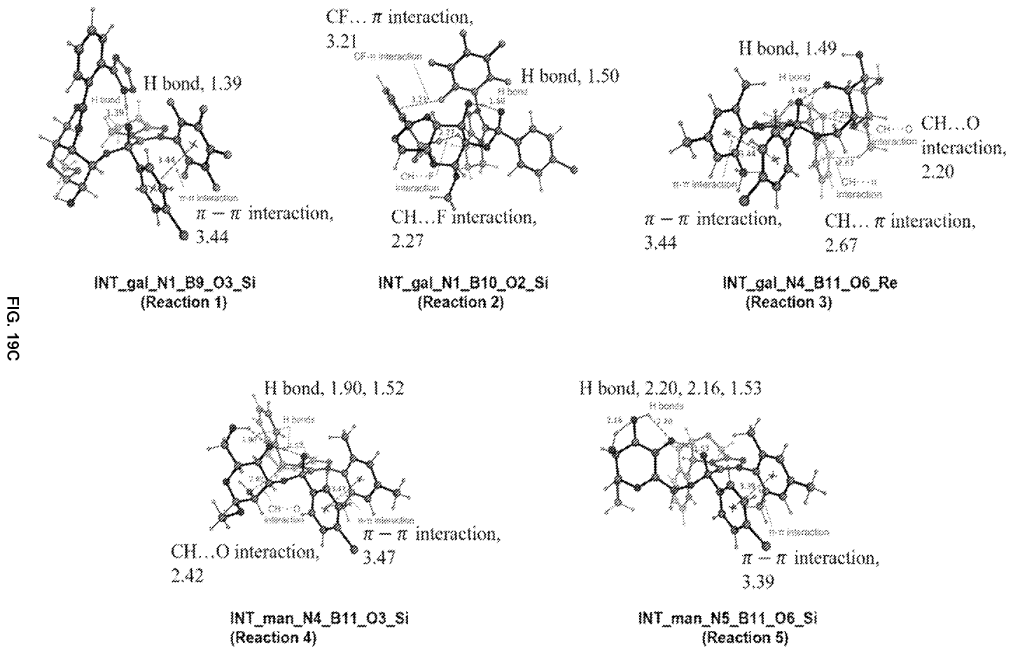

Our condition screening in this study ended up with the use of five NHC catalysts and eight boronic acids (with 5×8 possible combinations) for optimal outcomes of the different types of saccharides and acylation partners. Although a definite relation between structures and reaction outcomes cannot be drawn at this point, a number of guiding trends were observed, as illustrated in FIG. 5. For instance, the combination of N1 and B1 worked well for C(3)-OH acylation of α- and p-glucoside (combination 2 in FIG. 5). This combination (N1+B1) also worked effectively for similar selectivity patterns when we used carboxylic acids or esters as the acylation agents (FIG. 12). We eventually identified 12 combinations of NHCs and boronic acids (combinations 1-12 in FIG. 5) for various selective reactions on a large set of saccharides and acylation partners (FIGS. 11-18).

The substrate tolerances and limitations using aldehydes as the acylation reagents were studied (FIG. 11). With glucoside (1) as a model saccharide, C(3)-OH selective acylation could be achieved with various aryl aldehydes (3-17) and α,β-unsaturated aldehydes (18 and 19). The use of alkyl aldehyde gave little saccharide acylation adducts. Multiple different types of monosaccharides and their analogs could undergo selective C(3)-OH acylation as well (20-38). For example, β-glucosides and their derivatives, including a natural product (geniposide), could be selectively acylated (20-23) with 66%-75% yields and around 10:1 regioselectivity. Diabetes drugs containing analogs of monosaccharides, dapagliflozin and empagliflozin, could be acylated with good yields and excellent regioselectivity (24 and 25). The C(3)-OH acylation of α- and β-galactosides was achievable with NHC N1 and boronic acid B9 (combination 5) (26-32). Examples of other monosaccharides evaluated under current conditions for C(3)-OH acylations include aminoglycoside (33), α- and β-xylopyranosides (34 and 35), mannoside (36), rhamnopyranoside (37), and glucal (38).

Site-selective acylations on C(2)-OH moieties were obtained by a combination of N6+B8 (combination 11) or N1+B10 (combination 6) (FIG. 11). Examples of saccharides that gave satisfactory yields and selectivity values for C(2)-OH acylation under current conditions include glucoside and galactosides (39-46). The C(6)-OH of various saccharides and analogs (47-60) could be selectively acylated through the sole use of an NHC catalyst (combinations 1, 7, and 10) or in the presence of both NHCs and boronic acids (combinations 9 and 12). For example, the C(6)-OH of α- and β-glucosides was selectively acylated by NHC pre-catalyst N4 alone (47-50). Acylation on the C(6)-OH of β-galactosides (51-55) was realized by N4 and B11 (combination 9).

It is worth keeping in mind that for the same set of saccharide and acylation reagent, the use of different conditions offers dramatically different selectivity outcomes. For example, for the same aminoglycoside, the use of an NHC catalyst (N1) alone gave C(6)-OH acylation product 56, whereas a combined use of N1 and boronic acid B3 gave C(3)-OH acylation product 33. Similar comparisons can be made for other examples, such as products 3, 39, and 47 from α-glucoside (acylation on C3, C2, and C6, respectively). As a technical note, changes to both NHC catalysts and boronic acids are often needed for achieving optimal yields and selectivity values for each of the different OH groups on the same saccharides.

### Example 9. Effects of the various interactions between the components of the reaction on affect regioselective acylation

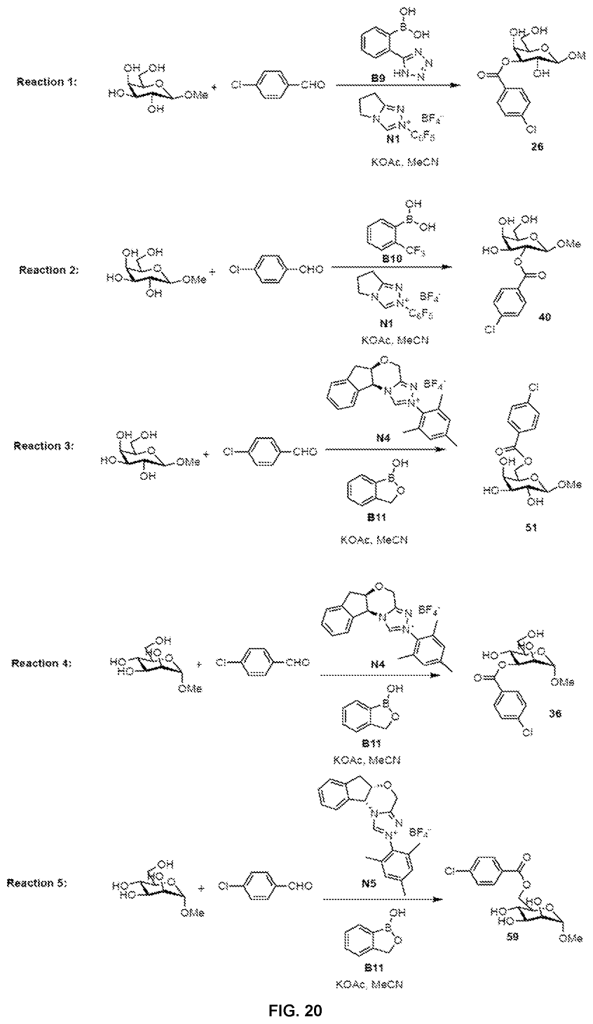

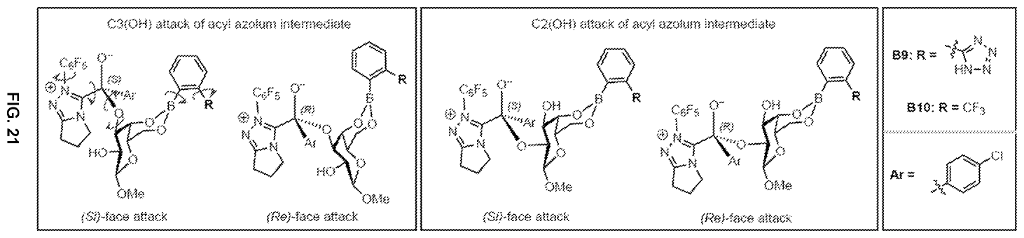

To understand how the various interactions between the components of the reaction affect regioselective acylation, we chose five model reactions to study (FIGS. 19 and 20) by focusing on the key regio-determining step via DFT calculations.

DFT calculations —Computational Methods

For conformational sampling of structures, Grimme's crest program (Grimme, S., J. Chem. Theory Comput. 2019, 15, 2847-2862; and Pracht, P., Bohle, F. & Grimme, S., Phys. Chem. Chem. Phys. 2020, 22, 7169-7192), which used metadynamics (MTD) with genetic z-matrix crossing (GC) performed at the GFN2-xTB (Bannwarth, C., Ehlert, S. & Grimme, S., J. Chem. Theory Comput. 2019, 15,1652-1671; Grimme, S., Bannwarth, C. & Shushkov, P., J. Chem. Theory Comput. 2017, 13, 1989-2009; and Bannwarth, C. et al., WIREs Comput Mol Sci. 2021, 11, e1493) extended semiempirical tight-binding level of theory, was used. The resulting lowest energy structures were further optimized using global hybrid DFT functional M06-2X (Grimme, S., J. Chem. Theory Comput. 2019, 15, 2847-2862) with Karlsruhe-family double-ζ valence def2-SVP (Weigend, F. & Ahlrichs, R., Phys. Chem. Chem. Phys. 2005, 7, 3297-3305; and Weigend, F., Phys. Chem. Chem. Phys. 2006, 8,1057-1065) basis set for all atoms as implemented in Gaussian 16 rev. B.01 (Frisch, M. J. et al., Gaussian 16, Revision B.01. 2016). Single point (SP) corrections were performed using M06-2X functional and def2-TZVP (Weigend, F. & Ahlrichs, R., Phys. Chem. Chem. Phys. 2005, 7, 3297-3305) basis set for all atoms. Minima and transition structures on the potential energy surface (PES) were confirmed as such by harmonic frequency analysis, showing respectively zero and one imaginary frequency. The implicit SMD continuum solvation model (Marenich, A. V., Cramer, C. J. & Truhlar, D. G., J. Phys. Chem. B 2009, 113, 6378-6396) for acetonitrile solvent was used to account for the effect of solvent on the potential energy surface. Gibbs energies were evaluated at 50° C., which was used in the experiments, using a quasi-RRHO treatment of vibrational entropies (Luchini, G. et al., F1000Research 2020, 9, 291). Vibrational entropies of frequencies below 100 cm⁻¹ were obtained according to a free rotor description, using a smooth damping function to interpolate between the two limiting descriptions (Grimme, S., Chem. Eur. J. 2012, 18, 9955-9964). The free energies were further corrected using standard concentration of 1 mol/L for gas-phase-to-solvent correction. All molecular structures are visualized using PyMOL software (Schr6dinger, L., The PyMOL molecular graphics development component, Version 1.8; 2015).

DFT calculations —Model Systems

To understand how the interactions between the NHC and the boronic acids employed effect the regioselective O-acylation, we chose the model reactions in FIG. 20 for our computational studies. (1) Comparing Reactions 1 and 2, we aimed to see how a difference in the substituent group in the boronic acid affects the regioselective outcome. (2) Comparing Reactions 3 and 4, which employ the same reaction conditions, except the sugar used, we aimed to understand how sugar stereochemistry affects regioselective outcome. (3) Finally, comparing Reactions 4 and 5, where enantiomeric NHCs are used on the same sugar, we aimed to understand how NHC chirality affects regioselective outcome. We considered the key step of the hydroxyl group attacking the carbonyl group of the acyl azolium as this step is regio-determining. The mechanistic study of the full catalytic cycle for the present reaction is underway in our laboratories.

Conformational analyses

To study the key regio-determining step of C-O bond formation between sugar hydroxyl group and the carbonyl C of acyl azolium intermediate, we need to consider the conformations of these TSs. As such TS structures could not be located at the xtb level, we considered the conformations of the key intermediates as a proxy to the conformations in the regio-determining TSs as we expect the side group interactions to be similar in the intermediate and the TSs. In other words, favorable interactions such as π-π interactions and hydrogen bonding interactions in the intermediates are expected to be also present in the TSs.

Conformational sampling of the acyl azolium-sugar intermediate was performed using the crest program, as outlined above. An implicit solvation of acetonitrile using the generalized Born (GB) model with surface area (SA) contribution (GBSA) was included in the conformational sampling. The lowest energy conformer from this procedure was further optimized at DFT SMD(acetonitrile)-M06-2X/def2-TZVP//M06-2X/def2-SVP level of theory.

Regio-determining TSs —case study using Reaction 4

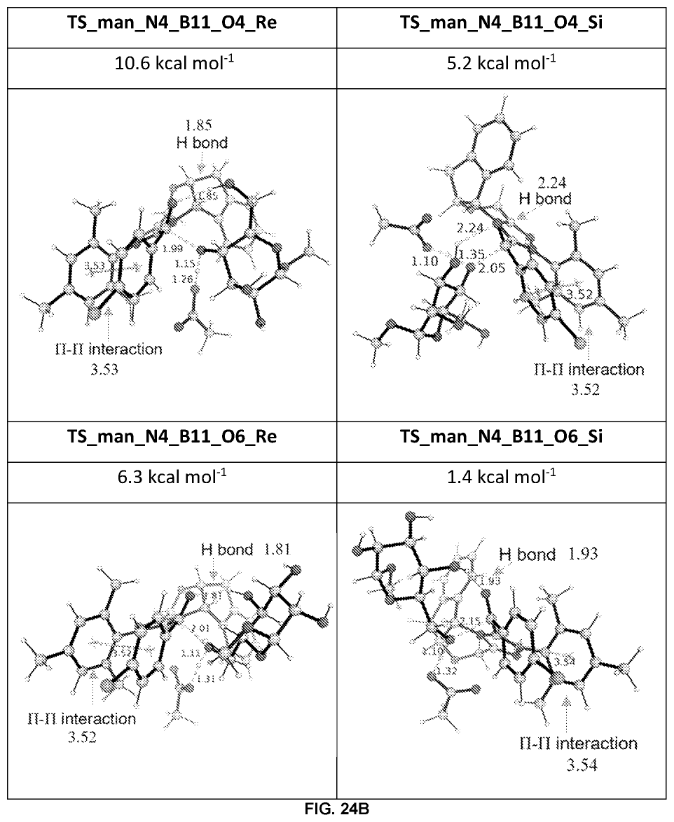

To verify that our usage of intermediates as a proxy to the interactions in the corresponding TSs is appropriate, we analyzed the TSs for the regio-determining step in Reaction 4 (FIG. 20). The optimized DFT TS structures are given in FIG. 24.

Results and discussion

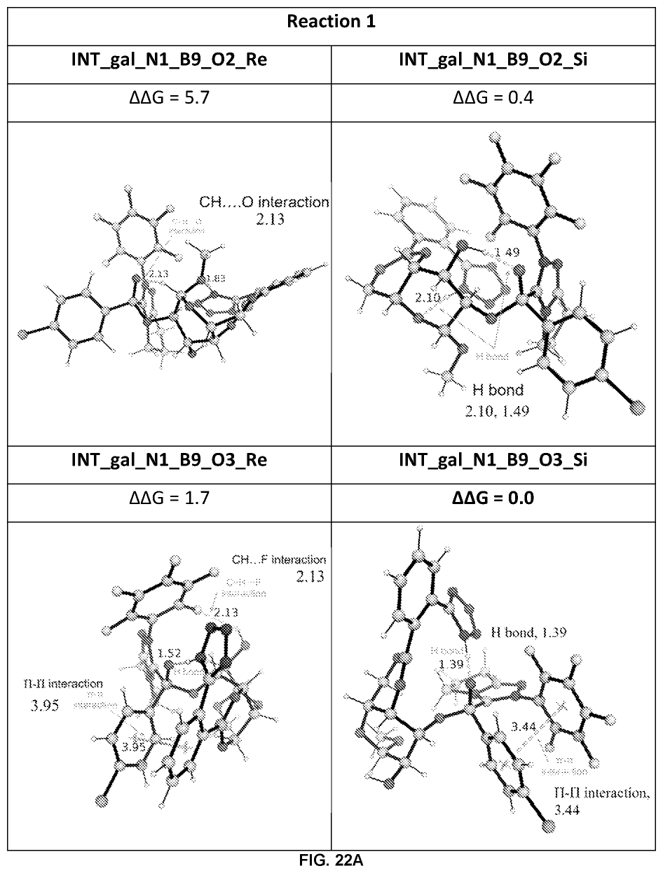

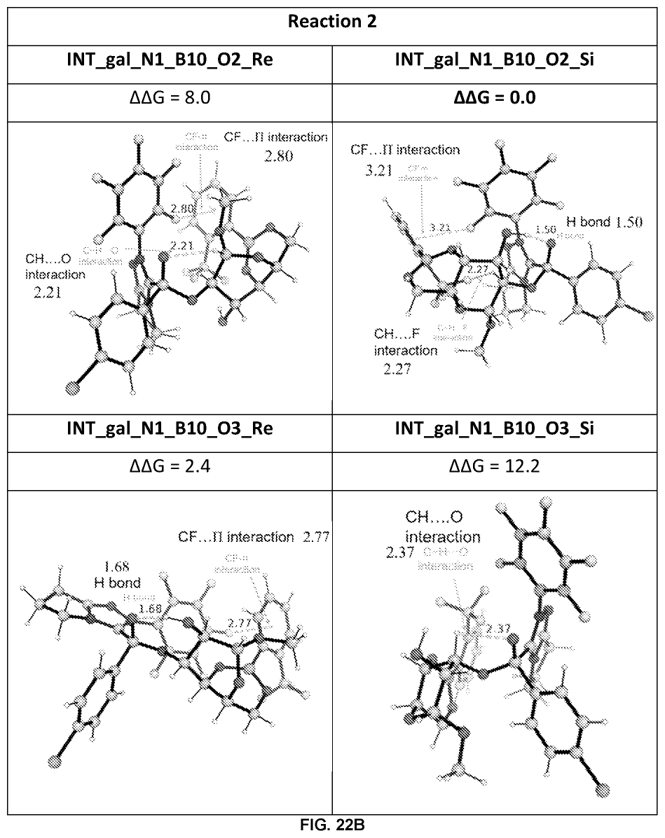

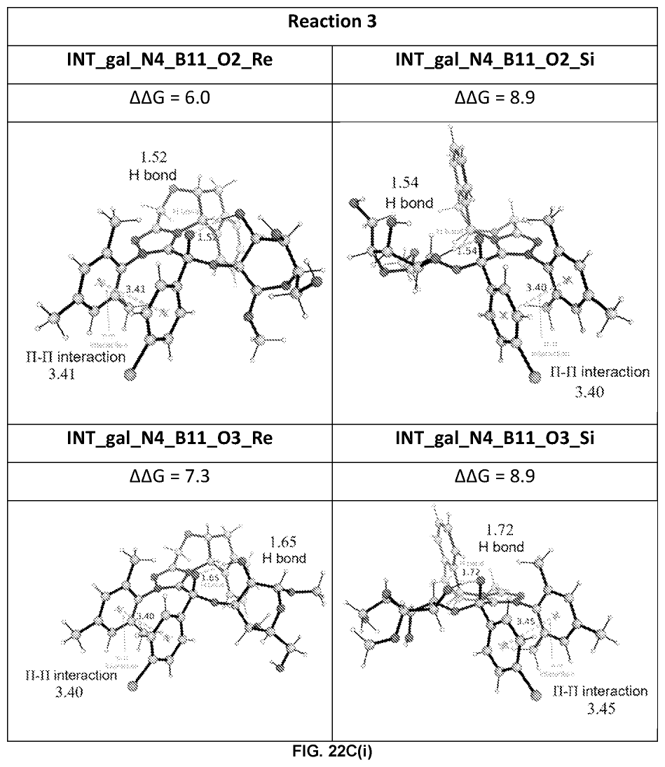

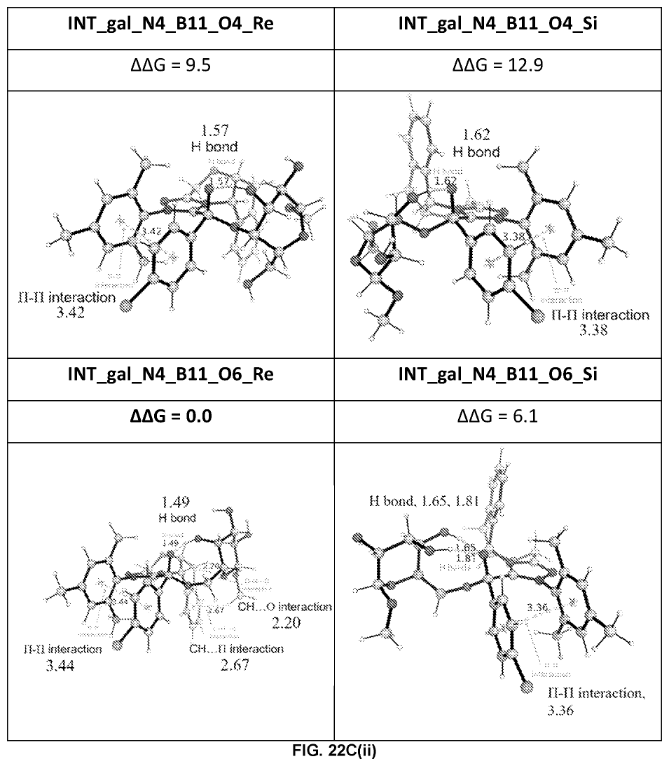

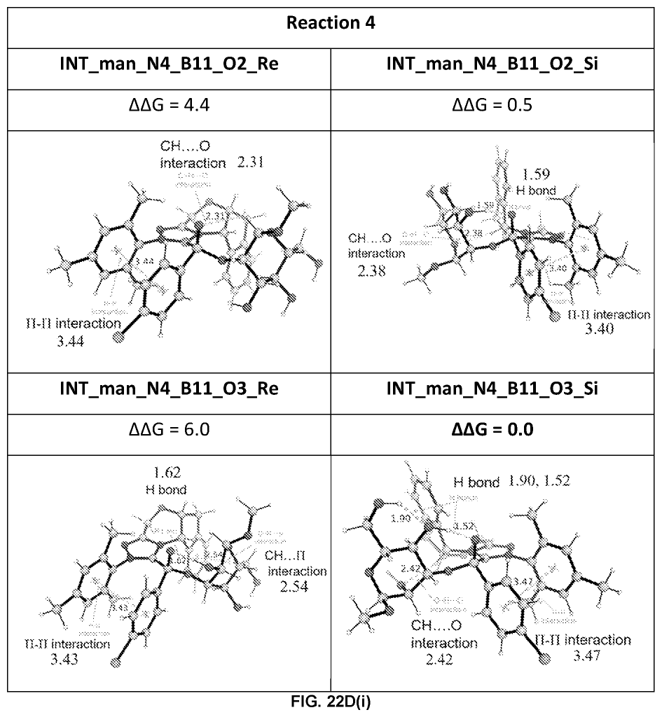

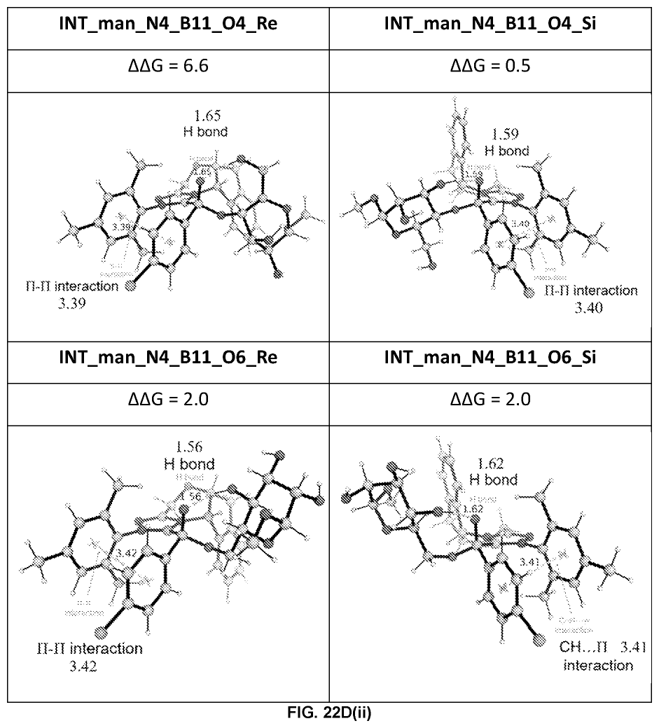

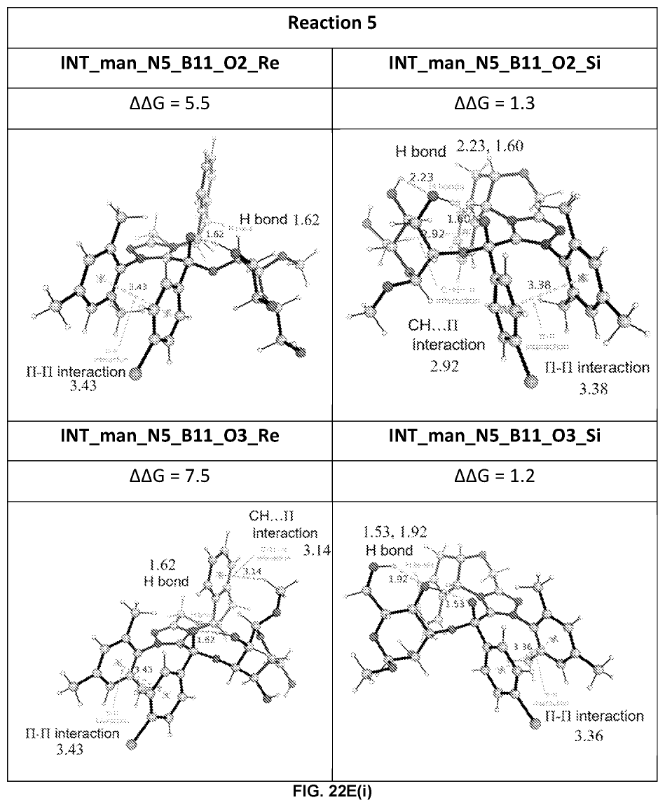

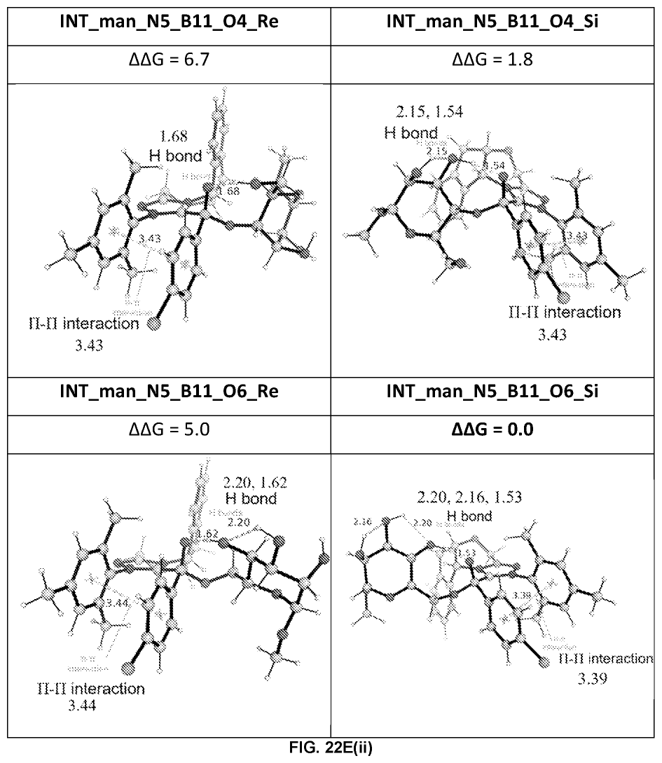

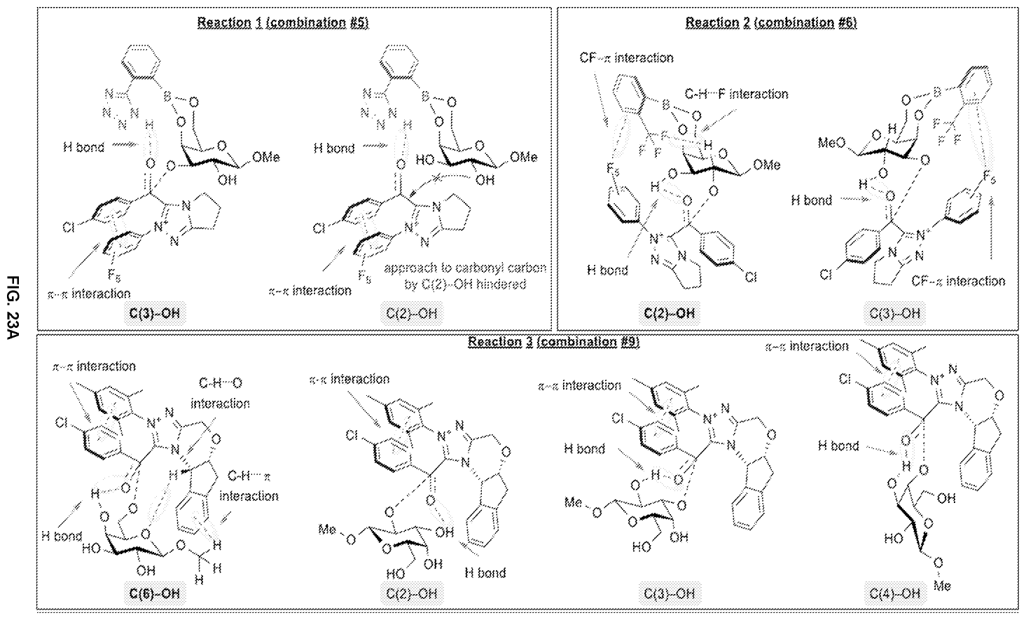

We aimed to discern how boronic acids (by comparing Reactions 1 and 2), monosaccharide identity and chirality (by comparing Reactions 3 and 4), and NHC chirality (by comparing Reactions 4 and 5) affect the site-selectivity outcomes. Given that the carbonyl carbon of the acyl azolium intermediate under attack by the monosaccharide is prochiral, allowing attack from either the (Re)-face or the (Si)-face by OH group (FIG. 21), we considered the regio-divergent intermediates arising from both possibilities. Within each reaction, independent conformational sampling converges to the lowest energy structures such that the same backbone orientation demonstrates similar interactions (FIG. 22).

FIG. 21 shows the examples of the intermediates arising from the attack of the acyl azolium carbonyl group by the hydroxyl groups from the boronic ester (sugar). Two possibilities can occur, namely that a particular OH group can attack the carbonyl group from either the (Si)-face or the (Re)-face, giving rise to different stereoisomers with differing interactions among the side groups. Note that, although the interactions in these intermediates, and their corresponding TSs, are different, the subsequent loss of NHC as the oxyanion reforms the carbonyl group generates the same acylated sugar product in each case.

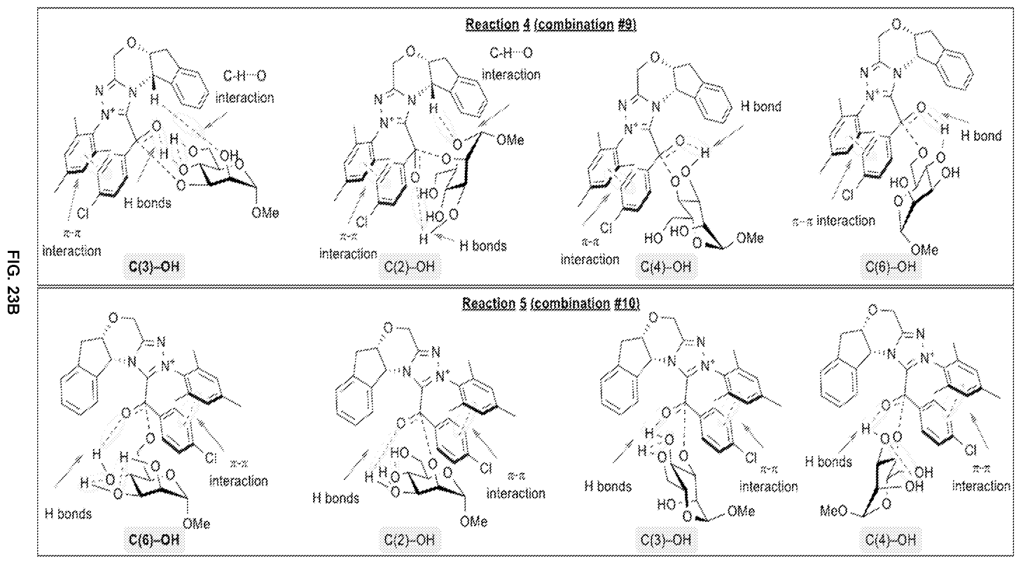

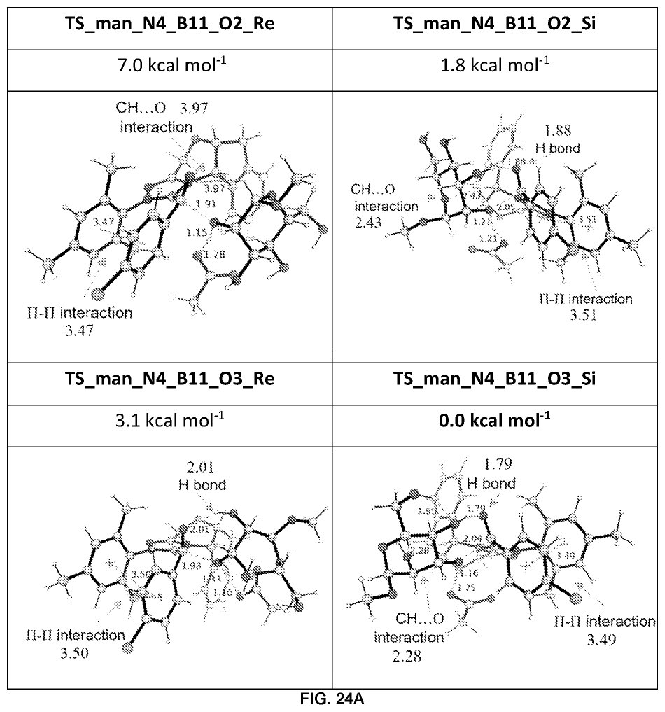

FIG. 22 shows the DFT optimized structures. In Reaction 1, the NH group of the tetrazole ring of the boronic acid can form hydrogen bonding interaction with the oxyanion oxygen atom in formation of O(C3)-C(carbonyl) from either the (Re)-(INT_gal_N1_B9_O3_Re) or the (Si)-face attack (INT_gal_N1_B9_O3_Si). We can imagine that the formation of this hydrogen bonding strategically places the C(3)-OH group close to the carbonyl C═O group for productive C-O bond formation in the transition state, as illustrated in FIG. 23. For the formation of O(C2) —C(carbonyl) bond however, no such hydrogen bonding is possible due to the geometric restraints. In INT_gal_N1_B9_O2_Si, instead, a hydrogen bonding between the NH group of the tetrazole ring of the boronic acid and the anomeric oxygen atom is formed. This is in addition to the hydrogen bonding between C(3)-OH and the oxyanion oxygen atom. In INT_gal_N1_B9_O2_Re, however, no such interactions are possible, and only weak CH---O interaction is possible, thus explaining its much higher energy. In addition, the intermediates at C(3)-OH functionalization have π-π interactions that are absent in the C(2)-OH functionalization. These could be the origins for favoring C(3)-OH functionalized galactoside using the combination of NHC N1 and boronic acid B9.

In Reaction 2, the most stable intermediate, INT_gal_N1_B10_O2_Si, benefits from various favorable interactions such as H bonding, CH—F and CF--π interactions. The H bond in this intermediate is stronger than the H bond in INT_gal_N1_B10_O3_Re (ΔΔG=2.4 kcal mol-1) as the former has a shortest distance of 1.50 Å than the latter of 1.68 Å (FIG. 22). On the other hand, only weak interactions are present in INT_gal_N1_B10_O2_Re and INT_gal_N1_B10_O3_Si (CH--O and CF---π interactions), and there is no H bonding present, thus giving much higher relative energies (by 8.0 and 12.2 kcal mol-1) than the most stable intermediate, INT_gal_N1_B10_O2_Si. Thus C(2)-OH functionalization of galactoside using the combination of NHC N1 and boronic acid B10 will be favored.

Comparing Reactions 1 and 2, we see that in Reaction 2, by changing the tetrazole ring of the boronic acid in Reaction 1 to trifluoromethyl group in Reaction 2, no H-bonding from the boronic acid moiety via the NH group of the tetrazole ring is possible in Reaction 2, thus, no directed “delivery” of C(3)-OH bond to the carbonyl group for addition is possible.

In Reactions 3, 4 and 5, the monosaccharides are not protected by forming 4,6-boronatomonosaccharides as the boronic acids do not have two OH groups. Therefore, we considered the possibility of functionalization at all OH groups on the sugar substrate. For each intermediate, our independent crest conformer search converges to the lowest energy structures with same backbone orientations demonstrating similar interactions. For example, the interactions between the NHC moiety and the aryl ring of the acyl group in INT_gal_N4_B11_Ox_Si (x=2, 3, 4, 6) are all the same; similar observation can be made in INT_gal_N4_B11_Ox_Re (x=2, 3, 4, 6). This demonstrates that within each reaction, the acyl azolium intermediate forms specific interactions, priming the carbonyl group for the regioselective addition of a particular OH group of the monosaccharide over other OH groups depending on the monosaccharide chirality and the specific interactions that the monosaccharide can form with the acyl azolium intermediate.

Looking at all the lowest energy intermediates from either the (Re)- or (Si)-face attack of the carbonyl group of the acyl azolium intermediate by various OH groups, we can see that all these structures form favorable π-π interactions between the aryl ring of the acyl group and the mesityl group on the NHC. For Reaction 3, the (Re)-face attacks give more stable intermediates than the corresponding (Si)-face attack at each C(OH) functionalization whereas for Reactions 4 and 5, due to the different stereochemical orientation of the sugar and the chiral NHC, the (Si)-face attacks give more stable intermediates than the corresponding (Re)-face attack.

In Reaction 3, comparing the intermediates of different O-site functionalization (INT_gal_N1_B10_Ox_Re where x=2, 3, 4, 6), we see that INT_gaI_N4_B11_O6_Re is the most stable, as this structure benefits from additional CH---O(anomeric) and CH-πinteractions that are not present in the other 3 intermediates (INT_gal_N1_B10_Ox_Re where x=2, 3, 4). In addition, although H-bonding between one of the OH groups on the monosaccharide and the oxyanion oxygen atom is formed in all cases, the H-bonding is the strongest in INT_gal_N4_B11_O6_Re as evidenced by its much shorter H-bond length of (1.49A) as compared to others (1.52 Å in INT_gal_N4_B11_O2_Re, 1.57 Å in INT_gal_N4_B11_O4_Re, and 1.65 Å in INT_gal_N4_B11_O3_Re). This suggests that the TS for the regio-determining C-O(C(6)-OH) bond formation will likely benefit from similar interactions and give the lowest energy barriers, thus suggesting that C(6)-OH acylation is the most likely.

In Reaction 4, as compared to Reaction 3, now the mannoside used has different stereochemistry than the galactoside at C(2)-OH and C(4)-OH. Now, the most stable intermediates, and by extension the corresponding TSs leading to their formation, result from the (Si)-face attacks rather than the (Re)-face attacks in Reaction 3. The intermediate formed at C(3)-OH, INT_man_N4_B11_O3_Si, is the most stable, as it has two H-bonds and additional CH---0 interaction and it has the strongest H-bond between the OH of manoside and oxyanion oxygen atom (bond distance of 1.52A, FIG. 22).

In Reaction 5, both the mannoside and the NHC have different stereochemistry from the galactoside and NHC used in Reaction 3. The most stable intermediates result from the (Re)-face attacks in Reaction 3, but from the (Si)-face attacks in Reaction 5. The double inversion of the stereochemistry in both the sugar and the NHC could explain why both Reactions 3 and 5 favor the same OH-functionalization (both at C(6)-OH). For example, comparing INT_gal_N4_B11_O6_Re and INT_man_N5_B11_O6_Si, the most stable intermediate in Reaction 3 and Reaction 5, respectively (FIG. 22), the dihydroindene group of the NHC in both cases have similar orientation (point “downwards”) as the sugar hydroxyl groups form various interactions. These two structures are almost mirror images, except where the stereochemistry of the sugar substrate differs. Both structures have the most favorable interactions than intermediates from other O-site functionalization within each of Reactions 3 and 5. The intermediate formed at C(3)-OH, INT_man_N4_B11_O3_Si, is the most stable, as it has two H-bonds and additional CH---O interaction and it has the strongest H-bond between the OH of mannoside and oxyanion oxygen atom (bond distance of 1.52 Å, FIG. 22).

When comparing Reaction 5 to Reaction 4, both the intermediates resulting from the (Si)-face attack of the acyl azolium have lower energy than the corresponding intermediates from the (Re)-face attack. Comparing the intermediates from the (Si)-face attack in Reactions 4 and 5 (FIG. 22), we see that only the orientation of the dihydroindene group of the NHCs differs across these two reactions (e.g., INT_man_N4_B11_Ox_Si vs INT_man_N5_B11_Ox_Si, where x=2, 3, 4). This is consistent with our expectation, as the NHCs used are enantiomers (N4 vs N5). This difference in the NHC side group orientation favors different O-functionalization (C(6)-OH in Reaction 5 vs C(3)-OH in Reaction 4 due to the resultant differing electronic and steric interactions present.

Within Reaction 5, the most stable intermediate is INT_man_N5_B11_O6_Si, at C(6)-OH functionalization. This intermediate forms three H-bonds whereas the other intermediates only have two H-bonds.

Therefore, comparing the TSs with their corresponding intermediates in FIG. 22, we can see that the same interactions present in the intermediates are also present in the TSs, thus suggesting that the stabilizing interactions giving stable intermediates also possibly stabilize the TSs.

In summary, the regioselective outcome of sugar O-functionalization results from a combination of sterics (due to side groups of the NHCs/boronic acids used) and electronic interactions between the sugar OH/CH groups and the NHC side chains. The acyl azolium intermediate is stereogenic as the carbonyl carbon can be attacked by sugar hydroxyl group from either the (Re)- or (Si)-face. This provides opportunities for unique interactions as different OH groups attack into the carbonyl carbon of acyl azolium, thus giving unique regioselective outcomes.

When boronic acid forms boronic ester by condensing with 4,6-diol of the monosaccharides, only the C(2)-OH and C(3)-OH groups are amenable to acylation (Table 4). This happens in Reactions 1 and 2. In Reaction 1, the NH group of the tetrazole ring of the boronic acid can form a hydrogen bond with the oxyanion oxygen atom. This formation of hydrogen bonding strategically places the C(3)-OH group close to the carbonyl C═O group for productive C-O bond formation (FIG. 19). For the formation of the O(C2)-C(carbonyl) bond, however, this approach is hindered by the geometric restraints. In Reaction 2, replacing the tetrazole in B9 with a trifluoromethyl group in B10 prevents the possibility of hydrogen bonding from the boronic acid moiety; instead, the CF3 group interacts with the acyl azolium differently to favor C(2)-OH over C(3)-OH acylation.

In Reactions 3-5, the monosaccharides are not protected by the formation of 4,6-boronato-monosaccharides because the boronic acids do not have two OH groups. The acyl azolium intermediates in these reactions each adopt a particular conformation stabilized by NCIs (FIG. 22), thus creating a specific site pocket, much akin to an enzyme's active site, for biased interaction with one particular OH group of the monosaccharide substrate over all other OH groups. In Reaction 3, C(6)-OH acylation would benefit from favorable interactions including additional CH---O(anomeric) and CH---π interactions that are not present in the other three intermediates. In Reaction 4, as compared with Reaction 3, a change in the sugar stereochemistry (mannoside versus galactoside with different chirality at C(2)-OH and C(4)-OH) leads the most stable intermediates to come from the (Si)-face attack rather than the (Re)-face attack in Reaction 3. Now, the intermediate formed at C(3)-OH is the most stable because it forms two hydrogen bonds and an additional CH---O interaction compared with the O-acylation at other sites. In Reaction 5, both the mannoside and the NHC have different stereochemistry from the galactoside and NHC used in Reaction 3. The most stable intermediates result from the (Re)-face attacks in Reaction 3 and from the (Si)-face attacks in Reaction 5. The double inversion of the stereochemistry in both the sugar and the NHC could explain why both Reactions 3 and 5 favor the same OH-functionalization (both at C(6)-OH). For example, when we compare the most stable intermediates in Reactions 3 and 5 (INT_gal_N4_B11_O6_Re and INT_man_N5_B11_O6_Si, respectively; FIG. 19), the dihydroindene groups of the NHC in both cases have orientations (pointing “downward”) similar to those of the sugar OH groups form various interactions. These two structures are almost mirror images, except where the stereochemistry of the sugar substrate differs. Both structures have the more favorable interactions than intermediates from other O-site functionalizations within each of Reactions 3 and 5. In Reactions 4 and 5, both the intermediates resulting from the (Si)-face attack of the acyl azolium have lower energy than the corresponding intermediates from the (Re)-face attack. When we compared the intermediates from the (Si)-face attack in Reactions 4 and 5 (INT_man_N4_B1 1_O3_Si and INT_man_N5_B11_O6_Si, respectively; FIG. 19), we see that only the orientation of the dihydroindene group of the NHCs differs across these two reactions. This is consistent with our expectation given that the NHCs used are enantiomers (N4 versus N5). This difference in the NHC side-group orientation favors different O-functionalization (C(6)-OH in Reaction 5 versus C(3)-OH in Reaction 4) as a result of the differing electronic and steric interactions.

Although this preliminary analysis of molecular interactions of various reaction components (NHC, boronic acid, and sugar) was performed on the regio-divergent intermediates, a similar analysis on the TSs using Reaction 4 lends validity to our current analysis, as we see that the same favorable interactions feature in both the intermediates and their corresponding TSs (compare FIG. 24 and Reaction 4 in FIG. 22). The TS barriers for Reaction 4 indicates that C(3)-OH acylation has the lowest activation barrier and is predicted to be kinetically most favorable, consistent with the experimentally observed C(3)-OH acylated product (FIG. 24).

An emerging theme from these DFT studies is that the regioselective outcome of sugar O-functionalization results from a combination of steric interactions (due to side groups of the NHCs and/or boronic acids used) and electronic interactions between the sugar OH and/or CH groups and the NHC and/or boronic acid side chains. The acyl azoliumintermediate is stereogenic because the carbonyl carbon can be attacked by the sugar OH group from either the (Re)- or (Si)-face. This provides opportunities for unique interactions that favor the functionalization of one OH group over all others given that the OH group attacks the carbonyl carbon of acyl azolium, thus giving unique regioselective outcomes.

The geometries of all optimized structures (in.xyz format with their associated energy in Hartrees) are included in a separate folder named final xyz structures. All these data have been uploaded to zenodo.org (DOI: 10.5281/zenodo.6327868).

Absolute values (in Hartrees) for SCF energy, zero-point vibrational energy (ZPE), enthalpy and quasi-harmonic Gibbs free energy (at 323.15K) for M06-2X/def2-SVP optimized structures are given in Table 9 below. Single point corrections in SMD(acetonitrile) using M06-2X/def2-TZVP functional are also included.

TABLE 9
Optimized structures and absolute energies, zero-point energies.

| Structure | E/au | ZPE/au | H/au | T.S/au | qh-G/au | SP M06-2X/def2TZVP |
|---|---|---|---|---|---|---|
| aldehyde_2a | −804.614711 | 0.101454 | −804.5037 | 0.042369 | −804.545817 | −805.1709337 |
| boronic_acid_B1 | −636.582931 | 0.192172 | −636.37469 | 0.057562 | −636.430201 | −637.3348694 |
| H2O | −76.323214 | 0.021594 | −76.297521 | 0.020204 | −76.317725 | −76.43444235 |
| NHC_N1 | −1084.927573 | 0.165762 | −1084.7433 | 0.063464 | −1084.804623 | −1086.209713 |
| AA_N1_c3 | −1888.769838 | 0.260864 | −1888.4813 | 0.085925 | −1888.561962 | −1890.656312 |
| AA_N1_c2 | −1888.775772 | 0.260836 | −1888.4873 | 0.084881 | −1888.567396 | −1890.656851 |
| AA_N1 | −1888.775793 | 0.260793 | −1888.4874 | 0.084921 | −1888.567482 | −1890.65731 |
| glucoside_1 | −725.643582 | 0.228916 | −725.39783 | 0.057656 | −725.454409 | −726.5196905 |
| glucoside_B1_23diol | −1209.552171 | 0.371438 | −1209.1532 | 0.084759 | −1209.232985 | −1210.966597 |
| glucoside_B1_34diol | −1209.54895 | 0.370831 | −1209.1503 | 0.085577 | −1209.230795 | −1210.965661 |
| glucoside_B1_46diol | −1209.568717 | 0.371562 | −1209.1697 | 0.084526 | −1209.249196 | −1210.982336 |
| galactoside | −725.652674 | 0.229552 | −725.40666 | 0.056875 | −725.462639 | −726.5227902 |
| B9 | −664.583683 | 0.154897 | −664.41454 | 0.053859 | −664.466841 | −665.3702877 |
| galactoside_B9_23diol | −1237.543626 | 0.333305 | −1237.1843 | 0.081445 | −1237.261347 | −1238.998131 |
| galactoside_B9_34diol | −1237.548714 | 0.332787 | −1237.1896 | 0.082703 | −1237.267408 | −1239.006574 |
| galactoside_B9_46diol | −1237.569034 | 0.33439 | −1237.2093 | 0.078775 | −1237.284232 | −1239.014657 |
| B10 | −744.489932 | 0.13184 | −744.34411 | 0.052736 | −744.395522 | −745.3714579 |
| galactoside_B10_23diol | −1317.449561 | 0.310053 | −1317.1136 | 0.080815 | −1317.19033 | −1318.998229 |
| galactoside_B10_34diol | −1317.463306 | 0.310522 | −1317.127 | 0.081408 | −1317.20371 | −1319.009917 |
| galactoside_B10_46diol | −1317.475216 | 0.310907 | −1317.139 | 0.079975 | −1317.214353 | −1319.020695 |
| mannoside | −725.64122 | 0.228852 | −725.39554 | 0.057456 | −725.452092 | −726.517678 |
| INT_gal_N1_B10_O2_Re | −3205.89074 | 0.56323 | −3205.2762 | 0.133772 | −3205.400908 | −3209.21994 |
| INT_gal_N1_B10_O2_Si | −3205.893766 | 0.561634 | −3205.2807 | 0.13518 | −3205.4059 | −3209.230654 |
| INT_gal_N1_B10_O3_Re | −3205.890674 | 0.562229 | −3205.2767 | 0.137606 | −3205.403266 | −3209.22644 |
| INT_gal_N1_B10_O3_Si | −3205.879828 | 0.563202 | −3205.2649 | 0.135125 | −3205.390642 | −3209.212477 |
| INT_gal_N1_B9_O2_Re | −3125.996992 | 0.586357 | −3125.3589 | 0.136407 | −3125.485177 | −3129.224536 |
| INT_gal_N1_B9_O2_Si | −3126.004327 | 0.585693 | −3125.3673 | 0.135464 | −3125.49276 | −3129.232675 |
| INT_gal_N1_B9_O3_Re | −3125.992594 | 0.585915 | −3125.3551 | 0.13613 | −3125.480944 | −3129.230787 |
| INT_gal_N1_B9_O3_Si | −3126.001052 | 0.584743 | −3125.3649 | 0.134511 | −3125.490114 | −3129.232745 |
| INT_gal_N4_B11_O2_Re | −2580.250711 | 0.698388 | −2579.5032 | 0.130524 | −2579.623753 | −2582.824691 |
| INT_gal_N4_B11_O2_Si | −2580.24786 | 0.698512 | −2579.5002 | 0.131113 | −2579.621057 | −2582.820042 |
| INT_gal_N4_B11_O3_Re | −2580.249975 | 0.699057 | −2579.5019 | 0.129462 | −2579.622199 | −2582.823436 |
| INT_gal_N4_B11_O3_Si | −2580.244493 | 0.699531 | −2579.4959 | 0.130745 | −2579.616763 | −2582.820846 |
| INT_gal_N4_B11_O4_Re | −2580.246036 | 0.698219 | −2579.4986 | 0.128967 | −2579.618901 | −2582.819359 |
| INT_gal_N4_B11_O4_Si | −2580.239243 | 0.697611 | −2579.492 | 0.130624 | −2579.613261 | −2582.812778 |
| INT_gal_N4_B11_O6_Re | −2580.266552 | 0.698464 | −2579.5196 | 0.126204 | −2579.638061 | −2582.835848 |
| INT_gal_N4_B11_O6_Si | −2580.251766 | 0.699229 | −2579.5037 | 0.128197 | −2579.623172 | −2582.826279 |
| INT_man_N4_B11_O2_Re | −2580.245541 | 0.697508 | −2579.498 | 0.132607 | −2579.620421 | −2582.823866 |
| INT_man_N4_B11_O3_Re | −2580.24213 | 0.697429 | −2579.495 | 0.130691 | −2579.616371 | −2582.822096 |
| INT_man_N4_B11_O4_Re | −2580.248702 | 0.698415 | −2579.501 | 0.130245 | −2579.621721 | −2582.822366 |
| INT_man_N4_B11_O6_Re | −2580.256535 | 0.698533 | −2579.5089 | 0.130708 | −2579.629701 | −2582.829512 |
| INT_man_N4_B11_O2_Si | −2580.259454 | 0.698493 | −2579.5118 | 0.129824 | −2579.632256 | −2582.832234 |
| NT_man_N4_B11_O3_Si | −2580.258888 | 0.698013 | −2579.5116 | 0.130913 | −2579.632655 | −2582.832067 |
| INT_man_N4_B11_O4_Si | −2580.255714 | 0.698096 | −2579.5082 | 0.132583 | −2579.630093 | −2582.83073 |
| INT_man_N4_B11_O6_Si | −2580.255623 | 0.698992 | −2579.5079 | 0.127901 | −2579.62715 | −2582.831125 |
| INT_man_N5_B11_O2_Re | −2580.24795 | 0.698137 | −2579.5004 | 0.130962 | −2579.621589 | −2582.825599 |
| INT_man_N5_B11_O3_Re | −2580.244282 | 0.697631 | −2579.4971 | 0.130823 | −2579.61835 | −2582.822082 |
| INT_man_N5_B11_O4_Re | −2580.253342 | 0.698956 | −2579.5055 | 0.128142 | −2579.625152 | −2582.825638 |
| INT_man_N5_B11_O6_Re | −2580.249624 | 0.698248 | −2579.5021 | 0.131858 | −2579.623538 | −2582.826188 |
| INT_man_N5_B11_O2_Si | −2580.25843 | 0.698057 | −2579.511 | 0.130152 | −2579.631821 | −2582.83253 |
| INT_man_N5_B11_O3_Si | −2580.25833 | 0.69756 | −2579.5114 | 0.130902 | −2579.632522 | −2582.831964 |
| INT_man_N5_B11_O4_Si | −2580.256943 | 0.698314 | −2579.5095 | 0.129624 | −2579.629921 | −2582.832262 |
| INT_man_N5_B11_O6_Si | −2580.260399 | 0.698206 | −2579.513 | 0.130915 | −2579.63397 | −2582.834486 |
| TS_man_N4_B11_O2_Re | −2809.076249 | 0.758052 | −2808.2625 | 0.14227 | −2808.395197 | −2811.915257 |
| TS_man_N4_B11_O3_Re | −2809.080915 | 0.757859 | −2808.2673 | 0.143717 | −2808.400763 | −2811.920635 |
| TS_man_N4_B11_O4_Re | −2809.07105 | 0.75845 | −2808.2571 | 0.142367 | −2808.38959 | −2811.909957 |
| TS_man_N4_B11_O6_Re | −2809.076314 | 0.759873 | −2808.261 | 0.144518 | −2808.394092 | −2811.917624 |
| TS_man_N4_B11_O2_Si | −2809.083887 | 0.758013 | −2808.2701 | 0.143355 | −2808.403344 | −2811.923044 |
| TS_man_N4_B11_O3_Si | −2809.085262 | 0.758016 | −2808.2714 | 0.145222 | −2808.405577 | −2811.925105 |
| TS_man_N4_B11_O4_Si | −2809.07664 | 0.759658 | −2808.2612 | 0.145359 | −2808.395015 | −2811.918819 |
| TS_man_N4_B11_O6_Si | −2809.082656 | 0.759436 | −2808.2678 | 0.143292 | −2808.400629 | −2811.925177 |

### Example 10. Carboxylic acids and esters as acylation reagents

Carboxylic acids and esters have a much bigger presence than aldehyde moieties in natural and synthetic bioactive molecules, such as pharmaceuticals; we therefore moved to employ acids and esters as the acylation reagents (FIG. 12).

Reactions with carboxylic acids and esters as the acylation reagents

A typical reaction condition using carboxylic acid is illustrated in FIG. 4 (optimal condition 2 Å). With a similar effort, conditions for using carboxylic esters (4-nitrophenol esters of carboxylic acids) could be readily realized, as exemplified by optimal condition 3 Å (FIG. 4).

Results and discussion

To our delight, the same set of NHC-boronic acid combinations offers nearly the same selectivity preference when carboxylic acids and esters are used. Only minor changes to the conditions such as solvents and bases are required. When carboxylic acids were used, a coupling agent (DCC) was used to convert the carboxylic acid to its reactive ester form for subsequent reaction with the NHC catalyst to form the NHC-bound acyl azolium intermediate (II; FIG. 4).

The reaction generality is exceptional with aryl or alkyl carboxylic acids and esters bearing various functional groups. For example, carboxylic acid-containing commercial pharmaceuticals reacted with monosaccharides in a highly regioselective manner to give the corresponding drug-saccharide conjugates (62-72, 83-84, and 95-96) with good isolated yields. Our reaction conditions are mild and tolerate sensitive functional groups, such as the endoperoxide 1,2,4-trioxane ring in artesunate (65 and 83). Carboxylic acid-containing amino acids, peptides, and their derivatives (73-82) were also excellent acylation partners under our approach. These results (73-82) suggest that our method could be further developed for the preparation of conjugates of saccharides and peptides or proteins. Our strategy could also be used to link two molecules with synergistic medicinal effects for possible combinatory therapeutics. Here, we show that two sophisticated bioactive molecules (dehydrocholic acid and geniposide) can be linked via saccharide selective acylation (72). Conjugation of paclitaxel with sugars has shown improved pharmaceutical properties (such as solubility and stability) and better target cancer cell specificity. Previous reported studies used the conventional protection-deprotection approach to link sugars to paclitaxel. Our method allows for concise access to glycoside-conjugated paclitaxel (95 and 96) in one step by using succinic acid as the linker. A number of carboxylic esters (3, 85, 39, 57, and 62; FIG. 12) were also examined as acylation reagents. Similarly, there are no apparent limitations with respect to the core scaffolds and substituents of the carboxylic esters.

### Example 11. Concise synthesis of complex molecules

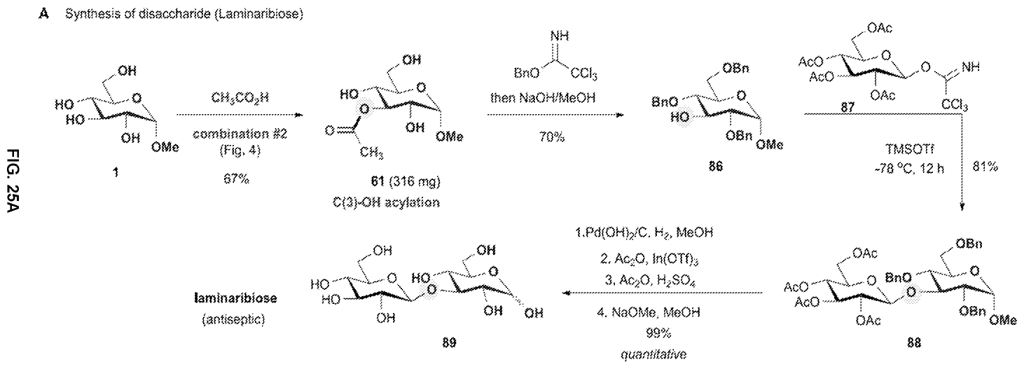

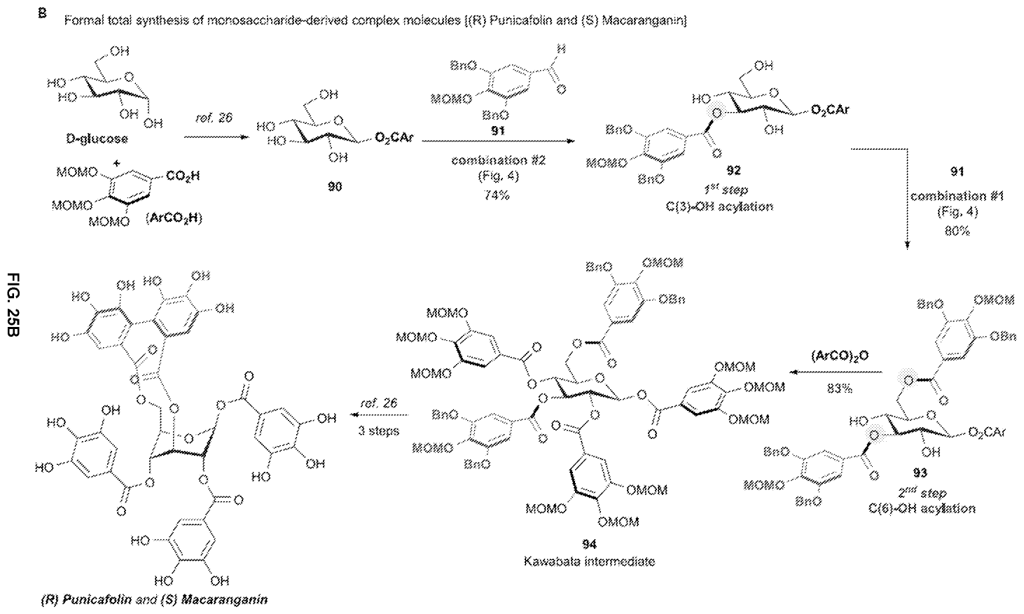

### Synthesis of disaccharide laminaribiose, related to FIG. 25A

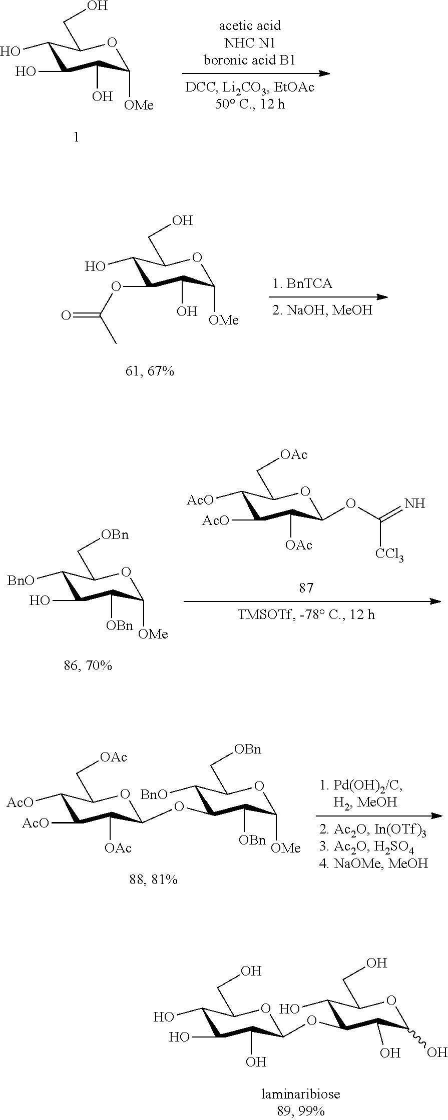

61

Monosaccharide 1 (2.0 mmol, 1.0 equiv), NHC N1 catalyst (20 mol %), boronic acid B1 (2.0 10 mmol, 1.0 equiv), DCC (4.0 mmol, 2.0 equiv), and Li₂CO₃ (4.0 mmol, 2.0 equiv) were added to a 100 mL flask. Then, EtOAc (40 mL) and carboxylic acid (4.0 mmol, 2.0 equiv) was added to the mixture. The reaction mixture was allowed to stir vigorously at 50° C. for 12 h under a N₂ atmosphere. After cooling to room temperature, the reaction mixture was filtered and concentrated to 15 mL, then directly purified by silica gel flash column chromatography with 15 an appropriate solvent (EtOAc/hexane 1:5 to 1:0 v/v) to afford the pure product 61 (316 mg, 67%).

### Methyl-2,4,6-O-benzyl-α-D-glucopyranoside (86)

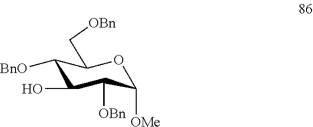

Trimethylsilyl trifluoromethanesulfonate (0.3 mmol) was added to a mixture of 61 (236 mg, 1.0 mmol), BnTCA (1.25 g, 5.0 mmol), powdered 4 Å molecular sieves (1g), and anhydrous dioxane (30 mL) at 0° C. The mixture was stirred at room temperature for 7 h under a N₂ atmosphere. Then, more BnTCA (2 mmol) and trimethylsilyl trifluoromethanesulfonate (0.05 mmol) was added and the reaction mixture was stirred for another 12 h. Then, the reaction mixture was concentrated and purified by silica gel flash column chromatography with an appropriate solvent (EtOAc/hexane 1:10 to 1:4 v/v) to afford the crude product. MeOH (10 mL) and NaOH (3.0 mmol) were added to the crude product. The reaction mixture was stirred at room temperature for 20 h. Then, the mixture was concentrated and purified by silica gel flash column chromatography with an appropriate solvent (EtOAc/hexane 1:10 to 1:4 v/v) to afford the pure product 86 (325 mg, 70%) as a colorless liquid.

¹H NMR (400 MHz, Acetone-d₆) δ 7.44 (d, J=7.1 Hz, 2H), 7.39-7.25 (m, 13H), 4.99 (d, J=11.3 Hz, 1H), 4.78 (d, J=3.5 Hz, 1H), 4.75-4.50 (m, 5H), 4.46 (s, 1H), 4.02 (t, J=9.2 Hz, 1H), 3.77-3.66 (m, 3H), 3.52-3.42 (m, 1H), 3.41-3.29 (m, 4H). ¹³C NMR (101 MHz, Acetone-d₆) δ 139.36, 139.23, 138.91, 128.16, 128.10, 128.03, 127.65, 127.65, 127.52, 127.31, 127.27, 127.17, 97.67, 80.11, 78.37, 74.20, 73.57, 72.86, 72.02, 70.11, 69.44, 54.25. ESI-MS: calcd for C₂₈H₃₂ONa [M+Na]⁺: 487.2097, found: 487.2087.

87

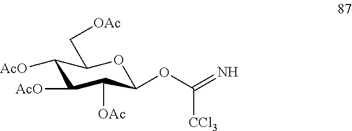

A solution of pentaacetate-D-glucose (10 mmol) and BnNH₂ (1.2 mL, 11 mmol) in THF (45 mL) was stirred at 50° C. for 18 h. The solvent was removed under reduced pressure and the residue was dissolved in CH₂C₁₂ and extracted with 10% HCl. The organic layer was concentrated and the residue was purified by flash column chromatography (1:5 EtOAc: Hexanes) to yield the anomerically deprotected tetraacetylated glucose. This product was redissolved in CH₂Cl₂ (120 mL) and cooled to 0° C. to which K₂CO₃ (30 mmol) and trichloroacetonitrile (50 mmol) were added and allowed to stir to ambient temperature over 18 h. The solution was filtered through celite and concentrated. The residue was purified by flash column chromatography to yield compound 87.

¹H NMR (400 MHz, CDCl₃) δ 2.02 (3H, s), 2.04 (3H, s), 2.06 (3H, s), 2.08 (3H, s), 4.13 (1H, dd, J=12.8, 2.0 Hz), 4.20-4.24 (1H, m), 4.28 (1H, dd, J=12.4, 4.0 Hz), 5.14 (1H, dd, J=10.0, 4.0 Hz), 5.19 (1H, t, J=10.0 Hz), 5.57 (1H, t, J=9.6 Hz), 6.57 (1H, d, J=4.0 Hz), 8.71 (1 H, s). ¹³C NMR (101 MHz, CDCl₃) δ 20.4, 20.5, 20.6, 61.3, 67.7, 69.6, 69.8, 69.9, 92.8, 160.7, 169.4, 169.8, 169.9, 170.5. ESI-MS: Calcd. for C₁₆H₂₀O₁₀NCl₃Na [M+Na]⁺: 514.0056. Found 514.0050.

### Methyl 2,4,6-tri-O-benzyl-3-O-(2,3,4,6-tetra-O-acetyl-β-D-glucopyranosyl)-α-D-glucopyranoside (88)

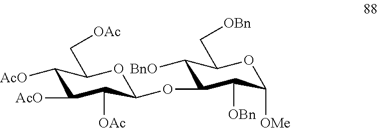

A solution of 86 (0.3 g, 0.646 mmol), 87 (476 mg, 0.97 mmol), and powdered 4 Å molecular sieves (0.6 g) in CH₂C₁₂ (12 mL) was cooled to −78° C. Then, trimethylsilyl trifluoromethanesulfonate (0.13 mmol) was added and the reaction mixture was stirred for 12 h at −78° C. under a N₂ atmosphere. When complete conversion of the starting material was observed, the reaction mixture was allowed to attain room temperature, concentrated, and purified by silica gel flash column chromatography with an appropriate solvent (EtOAc/hexane 1:10 to 1:3 v/v) to afford the pure product 88 (416 mg, 81%) as a colorless liquid.

¹H NMR (400 MHz, Acetone-d₆) δ 7.54 (d, J=7.3 Hz, 2H), 7.48-7.20 (m, 13H), 5.38-5.23 (m, 2H), 5.15-4.99 (m, 3H), 4.83-4.74 (m, 2H), 4.69 (d, J=11.4 Hz, 1H), 4.60-4.47 (m, 3H), 4.35-4.16 (m, 2H), 4.08 (dd, J=12.3, 2.6 Hz, 1H), 3.89 (ddd, J=10.1, 4.6, 2.5 Hz, 1H), 3.72-3.61 (m, 3H), 3.58-3.45 (m, 2H), 3.34 (s, 3H), 2.06 (s, 3H), 2.00 (s, 3H), 1.97 (s, 3H), 1.93 (s, 3H). ¹³C NMR (101 MHz, Acetone-d₆) δ 169.83, 169.43, 169.13, 168.92, 139.26, 138.79, 138.76, 128.31, 128.20, 128.14, 127.94, 127.94, 127.65, 127.60, 127.35, 127.20, 100.45, 97.06, 81.00, 79.09, 75.98, 74.21, 72.91, 72.79, 72.24, 71.86, 71.53, 69.95, 69.16, 68.60, 62.03, 54.26, 19.93, 19.74, 19.71, 19.65. ESI-MS: calcd for C₄₂H₅₀O₁₅Na [M+Na]⁺: 817.3047, found: 817.3050.

### Laminaribiose (89) (Kitaoka, M., Sasaki, T. & Taniquchi, H., J. JPn. Soc. Starch Sci. 1993, 40, 311-314)

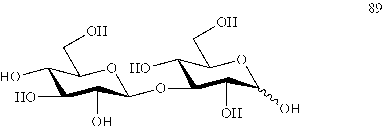

88 (794 mg, 1 mmol) in MeOH (15 mL) was stirred with Pd(OH)₂/C (700 mg) and H₂ (1 atm) for 24 h at 25° C. Then, the reaction mixture was filtered and concentrated. The crude product was treated with Ac₂O(3 mL), then In(OTf)₃ (0.1 mmol) was added to the mixture at 0° C. The reaction mixture was stirred for 3 h at 25° C. under a N₂ atmosphere. Then, another portion of Ac₂O(12 mL) and H₂SO₄ (250 μL) were added to the mixture successively at 0° C. The reaction mixture was stirred at 0° C. for another 6 h, then poured onto ice. The mixture was extracted with EtOAc, the extracts were washed with water, saturated NaHCO₃, saturated NaCl and dried with Na₂SO₄. Concentration of the organic extract gave acetyl-2,4,6-tri-O-acetyl-3-O-(2,3,4,6-tetra-O-acetyl-β-D-glucopyranosyl)-D-glucopyranoside as a colourless oil and as a mixture of anomers (680 mg, 99%, α:β, 7:1). NaOMe (0.1 mmol) in MeOH (1 mL) was added to acetyl-2,4,6-tri-O-acetyl-3-O-(2,3,4,6-tetra-O-acetyl-β-D-glucopyranosyl)-D-glucopyranoside (0.3 mmol) in MeOH (4 mL) at 0° C. and the solution was stirred at 25° C. for 4 h. Then the solution was directly purified by silica gel flash column chromatography with an 10 appropriate solvent (EtOAc/MeOH 1:0 to 1:1 v/v) to afford laminaribiose 89 (103 mg, 99%) as a white solid.

¹H NMR (400 MHz, D₂O) δ 5.15 (d, J=3.8 Hz, 1H), 4.66-4.56 (m, 2H), 3.92-3.04 (m, 19H). 13C NMR (101 MHz, D₂O) δ (102.93, 102.84), 95.70, 92.03, 84.72, 82.46, (76.04, 76.01), 15 75.59, (73.80, 73.51, 73.48), (71.24, 71.02), 69.60, (68.19, 68.15), (60.71, 60.58). ESI-MS: calcd for C₁₂H₂₂O₁₁Na [M+Na]⁺: 365.1060, found: 365.1064.

### Methyl-3-O-(2,3,4,6-tetra-O-acetyl-β-D-glucopyranosyl)-α-D-glucopyranoside (89-1)

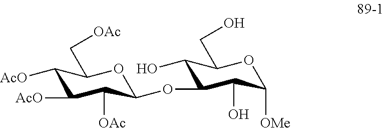

¹H NMR (400 MHz, Chloroform-d) δ 5.24 (t, J=9.6 Hz, 1H), 5.09-4.94 (m, 2H), 4.75 (d, J=3.2 Hz, 1H), 4.65 (d, J=8.0 Hz, 1H), 4.22 (d, J=12.0 Hz, 1H), 4.11 (dd, J=12.2, 6.6 Hz, 1H), 3.93-3.73 (m, 4H), 3.64-3.48 (m, 4H), 3.43 (s, 3H), 2.40 (s, 2H), 2.08 (s, 3H), 2.05 (s, 3H), 2.03 (s, 3H), 2.00 (s, 3H). ¹³C NMR (101 MHz, Chloroform-d) δ 170.62, 170.11, 169.97, 169.46, 102.43, 99.19, 87.68, 72.29, 71.88, 71.32, 71.08, 70.67, 69.24, 69.22, 68.53, 62.38, 62.33, 62.03, 55.28, 20.64, 20.53. ESI-MS: calcd for C₂₁H₃₂O₁₅Na [M+Na]⁺: 547.1639, found: 547.1638.

### Acetyl-2,4,6-tri-O-acetyl-3-O-(2,3,4,6-tetra-O-acetyl-β-D-glucopyranosyl)-D-glucopyranoside (89-2)

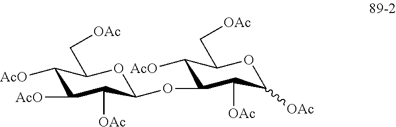

The product 89-2 (680 mg, 99%) was obtained as a colorless liquid.

R_{F} (hexane:ethyl acetate=1:2): 0.37. ¹H NMR (400 MHz, Chloroform-d) δ 6.25 (d, J=3.8 Hz, 1H), 5.25-5.00 (m, 4H), 4.91 (t, J=8.7 Hz, 1H), 4.66 (d, J=8.1 Hz, 1H), 4.49-4.33 (m, 1H), 4.26-4.01 (m, 5H), 3.75 (ddd, J=9.7, 3.9, 2.5 Hz, 1H), 2.32-1.88 (m, 24H). (H NMR (400 MHz, Chloroform-d) δ 5.63 (d, J=8.4 Hz, 1H), 5.25-5.00 (m, 4H), 4.91 (dd, J=9.3, 8.1 Hz, 1H), 4.61 (d, J=8.1 Hz, 1H), 4.49-4.33 (m, 1H), 4.26-4.01 (m, 4H), 3.95 (t, J=9.4 Hz, 1H), 3.75 (ddd, J=9.7, 3.9, 2.5 Hz, 1H), 2.32-1.88 (m, 24H).). ¹³C NMR (101 MHz, Acetone-d6) δ 169.82, 169.79, 169.68, 169.43, 169.00, 168.98, 168.77, 168.63, 100.47, 88.77, 76.29, 72.77, 71.41, 71.29, 71.19, 69.86, 68.24, 67.45, 61.69, 61.63, 19.88, 19.87, 19.79, 19.74, 19.69, 19.67, 19.60, 19.52. ESI-MS: calcd for C₂₆H₃₅O₁₇ [M-OAc]⁺: 619.1874, found: 619.1899.

### Formal total synthesis of punicafolin and macaranganin, related to FIG. 25B

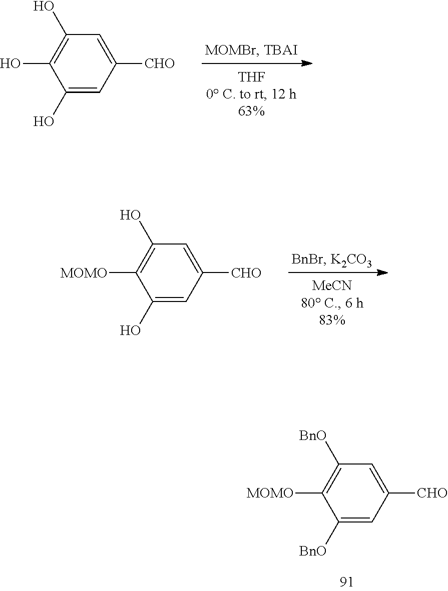

### 3,5-dihydroxy-4-(methoxymethoxy)benzaldehyde

MOMBr (7.15 mmol, 1.2 equiv) was added dropwise to a solution of 3,4,5-trihydroxybenzaldehyde (918 mg, 5.96 mmol, 1 equiv), tetra-n-butylammonium iodide (TBAI, 1.79 mmol, 0.3 equiv) and DIPEA (7.15 mmol, 1.2 equiv) in dry THF (60 mL) at 0° C. Then, the reaction mixture was warmed to room temperature and stirred at room temperature for 12 h. The reaction was quenched by saturated NaHCO₃ aqueous solution, and extracted by EtOAc (60 mL×3). The organic layers were combined, dried over Na₂SO₄ and concentrated. The crude mixture was purified by flash column chromatography on silica with an appropriate solvent to afford 3,5-dihydroxy-4-(methoxymethoxy)benzaldehyde (741 mg, 63%) as a yellow solid.

### 3,5-bis(benzyloxy)-4-(methoxymethoxy) benzaldehyde (91)

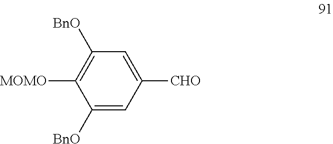

K₂CO₃ (1.70 mmol, 3 equiv) and benzyl bromide (1.25 mmol, 2.2 equiv) were added to a solution of 3,5-dihydroxy-4-(methoxymethoxy)benzaldehyde (112 mg, 0.57 mmol, 1 equiv) in dry MeCN (6 mL). Then, the reaction mixture was stirred at 80° C. for 6 h. After cooling to room temperature, the solution was filtered and concentrated. The crude mixture was purified by flash column chromatography on silica with an appropriate solvent to afford 91 (178 mg, 83%) as a white solid.

¹H NMR (400 MHz, Chloroform-d) δ 9.80 (s, 1H), 7.52-7.28 (m, 10H), 7.19 (s, 2H), 5.24 (s, 2H), 5.16 (s, 4H), 3.49 (s, 3H). ¹³C NMR (101 MHz, Chloroform-d) δ 190.93, 153.12, 141.36, 136.26, 132.08, 128.67, 128.22, 127.53, 108.50, 98.38, 71.21, 57.36. ESI-MS: calcd for C₂₃H₂₃O₅[M+H]⁺: 379.1545, found: 379.1546.

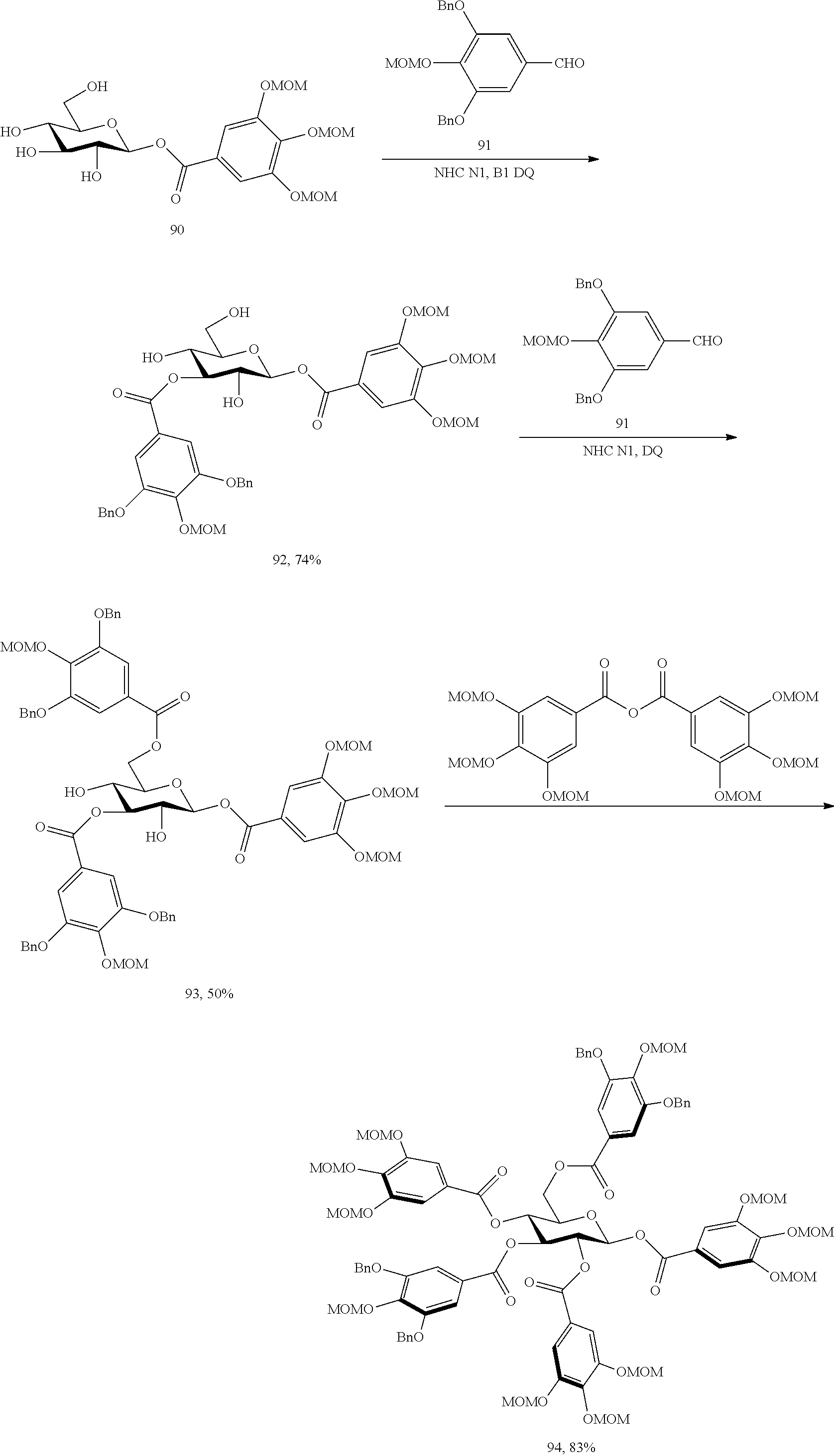

90

90 and acid anhydride were prepared according to the reference (Shibayama, H. et al., J. Am. Chem. Soc. 2021, 143, 1428-1434).

### 3,4,5-tris(methoxymethoxy)benzoyl-3-O-(3,5-bis(benzyloxy)-4-(methoxymethoxy) benzoyl)-α-Dalucopyranoside (92)

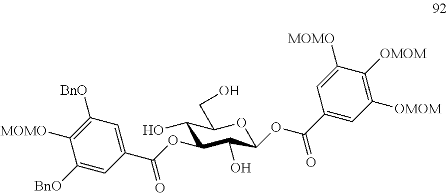

90 (0.1 mmol, 1.0 equiv), aldehyde 91 (0.2 mmol, 2.0 equiv), NHC N1 (10 mol %), boronic acid B1 (0.1 mmol, 1.0 equiv), DQ (0.2 mmol, 2.0 equiv), and K₂CO₃ (0.02 mmol, 0.2 equiv) were added to a 4 mL screwtop test tube. Then, acetonitrile (2 mL) was added to the mixture. The reaction mixture was allowed to stir vigorously at room temperature for 24 h under a N₂ atmosphere. Then, the reaction mixture was directly purified by flash column chromatography on silica with an appropriate solvent to afford 92 (62 mg, 74%; 84% brsm) as a colorless gum.

¹H NMR (400 MHz, Chloroform-d) δ 7.59 (s, 2H), 7.49-7.43 (m, 6H), 7.42-7.37 (m, 4H), 7.36-7.31 (m, 2H), 5.87 (d, J=8.1 Hz, 1H), 5.29-5.13 (m, 13H), 4.03-3.79 (m, 4H), 3.61 (s, 4H), 3.50 (d, J=6.8 Hz, 9H), 3.20 (d, J=25.0 Hz, 2H), 2.33 (s, 1H). ¹³C NMR (126 MHz, Chloroform-d) δ 167.34, 164.39, 152.46, 150.78, 141.40, 140.58, 136.51, 128.61, 128.14, 127.64, 124.55, 124.43, 111.92, 109.21, 98.51, 98.31, 95.16, 94.82, 79.48, 76.39, 71.42, 71.27, 68.94, 61.82, 57.31, 57.26, 56.45. ESI-MS: calcd for C₄₂H₄₈O₁₈Na [M+Na]⁺: 863.2738, found: 863.2744.

### 3,4,5-tris(methoxymethoxy)benzoyl-3,6-bis-O-(3,5-bis(benzyloxy)-4-(methoxymethoxy) benzoyl)-β-D-glucopyranoside (93)

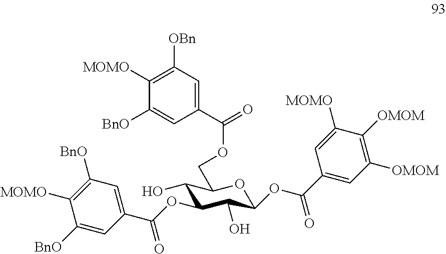

92 (45 mg, 0.054 mmol, 1.0 equiv), aldehyde 91 (0.108 mmol, 2.0 equiv), NHC N1 (10 mol %), DQ (0.108 mmol, 2.0 equiv), and DBU (0.011 mmol, 0.2 equiv) were added to a 4 mL screwtop test tube. Then, acetonitrile (1 mL) was added to the mixture. The reaction mixture was allowed to stir vigorously at room temperature for 24 h under a N₂ atmosphere. Then, the reaction mixture was directly purified by flash column chromatography on silica with an appropriate solvent to afford 93 (52 mg, 80%) as colorless gum.

¹H NMR (500 MHz, Chloroform-d) δ 7.55 (s, 2H), 7.42 (dt, J=7.3, 3.2 Hz, 11H), 7.38-7.26 (m, 13H), 5.89 (d, J=8.1 Hz, 1H), 5.26 (t, J=9.3 Hz, 1H), 5.23-5.05 (m, 18H), 4.67 (dd, J=12.4, 5.0 Hz, 1H), 4.57 (dd, J=12.3, 2.3 Hz, 1H), 3.98 (t, J=8.8 Hz, 1H), 3.89 (ddd, J=9.9, 5.0, 2.4 Hz, 1H), 3.71 (t, J=9.5 Hz, 1H), 3.58 (s, 3H), 3.55 (s, 1H), 3.49-3.37 (m, 12H), 3.06 (s, 1H). ¹³C NMR (126 MHz, Chloroform-d) δ 167.16, 166.76, 164.30, 152.44, 152.38, 150.76, 141.39, 140.63, 140.31, 136.51, 136.48, 128.60, 128.56, 128.15, 128.07, 127.65, 124.75, 124.61, 124.45, 111.89, 109.29, 109.03, 98.51, 98.35, 98.33, 95.14, 94.93, 78.80, 75.32, 71.41, 71.29, 71.13, 68.93, 63.69, 57.31, 57.27, 57.25, 56.42. ESI-MS: calcd for C₆₅H₆₆O₂₃Na [M+Na]⁺: 1239.4049, found:1239.4053.

### 1,2,4-Tris-O-[3,4,5-tris(methoxymethoxy)benzoyl-3,6-bis-O-[3,5-dibenzyloxy-4-(methoxymethoxy)benzoyl-β-D-glucopyranoside (94)

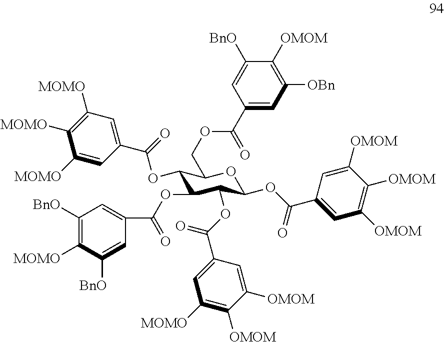

Monosaccharide 93 (40 mg, 0.033 mmol, 1.0 equiv), acid anhydride (0.132 mmol, 2.0 equiv), DMAP (0.033 mmol, 1 equiv) and acetonitrile (0.8 mL) were added to a 4 mL screwtop test tube. The reaction mixture was allowed to stir vigorously at 50° C. for 24 h under a N₂ atmosphere. After cooling to room temperature, the reaction mixture was directly purified by flash column chromatography on silica with an appropriate solvent to afford 94 (49 mg, 83%) as a colorless gum.

¹H NMR (500 MHz, Acetone-d₆) δ 7.62-7.21 (m, 30H), 6.48 (d, J=8.3 Hz, 1H), 6.21 (t, J=9.7 Hz, 1H), 5.96 (t, J=9.7 Hz, 1H), 5.84 (dd, J=9.9, 8.3 Hz, 1H), 5.31-5.16 (m, 22H), 5.13 (s, 2H), 5.07 (s, 6H), 4.89 (dd, J=12.5, 2.2 Hz, 1H), 4.79 (ddd, J=10.0, 4.8, 2.4 Hz, 1H), 4.44 (dd, J=12.5, 4.6 Hz, 1H), 3.55 (d, J=1.4 Hz, 6H), 3.51 (s, 3H), 3.49-3.40 (m, 21H), 3.35 (s, 3H). ¹³C NMR (126 MHz, Acetone-d₆) δ 165.84, 165.80, 165.68, 165.49, 164.54, 153.36, 153.26, 151.94, 151.93, 151.88, 142.87, 142.76, 142.71, 141.22, 141.02, 138.00, 137.63, 129.43, 129.31, 128.90, 128.87, 128.59, 125.93, 125.32, 125.23, 125.19, 124.66, 112.73, 112.70, 112.64, 109.41, 109.37, 99.12, 99.10, 99.08, 98.94, 98.81, 96.15, 96.12, 96.01, 93.77, 74.08, 73.70, 72.59, 71.69, 71.45, 70.41, 63.34, 57.26, 57.24, 57.22, 57.19, 57.17, 56.60, 56.53, 56.50. ESI-MS: calcd for C₉₁H₁₀₀O₃₇Na [M+Na]⁺: 1807.5841, found:1807.5846.

Results and discussion

Our site-selective acylation of monosaccharides enables the concise synthesis of complex molecules such as oligosaccharides and functional molecules containing saccharide fragments and their derivatives (FIG. 25). For example, starting from C(3)-OH acylated glucoside adduct (61) prepared by our strategy, antiseptic disaccharide laminaribiose could be prepared via a few straightforward operations in 38% overall yield from commercially available glucoside 1, as illustrated in FIG. 25A. It is reasonable to expect that by varying the reactive site (such as C(3)-OH of 1) and the monosaccharide coupling units (such as 87), our method should allow for rapid access to a diverse set of useful disaccharides and their analogs 15 (Nicotra, F. et al., J. Carbohydr. Chem. 1992, 11, 397-399), including those that are expensive and difficult to obtain. Our method can also allow for efficient synthesis of saccharide-derived complex molecules (FIG. 25B).

As a technical note, because many NHC catalysts and boronic acids are commercially available or easily accessible, further improvements in reaction efficiency and alternative site selectivity are readily achievable by our strategy. Molecular libraries of these natural products and their analogs can most likely be prepared in scalable quantities for bioactivity evaluations.

In summary, we have developed a readily programmable strategy for site-selective acylation of unprotected monoglycosides. The selectivity was achieved by proper combinations of commercially available NHC organic catalysts and boronic acids. The synergistic activation and deactivation effects brought by the NHC and boronic acid dramatically amplify the reactivity difference of the multiple otherwise similar OH groups on saccharides. Such synergistic effects can also invert the initial reactivity preference of these OH moieties, offering selectivity patterns that are not available with previous strategies. Our approach can selectively acylate the C(2)-, C(3)-, and (C6)-OH groups of various monosaccharides and their analogs. Aldehydes, carboxylic acids, and carboxylic esters can all be used as the acylation reagents. We have also demonstrated that carboxylic acid- or saccharide-containing pharmaceuticals, peptides, natural products, and other functional molecules can be site-selectively modified by our strategy. Application of our site-selective reaction can allow for concise and scalable access to such complex molecules as disaccharides and bioactive natural products. Given the unarguable significance and challenges associated with saccharides, we expect our approach to offer both fundamental and practical impacts in broad fields ranging from chemistry to medicine. Ongoing studies in our laboratories include site-selective reactions of complicated oligosaccharides, concise synthesis of sophisticated molecules bearing saccharide fragments, and bioactivity evaluation of saccharide-containing bioactive molecules for medicinal and agricultural applications.

### Comparative Example 1

Here, we demonstrate a formal total synthesis of puncafolin and macaranganin, natural products of the ellagitannin family, containing a monosaccharide core with important bioactivities. The first total synthesis of these two natural products was recently reported and in the reported approach, sequential selective acylations at the C(4)- and C(2)-OH groups (of 90) as mediated by Kawabata's elegant pyrrolidinopyridine-based catalysts are key steps in preparing intermediate 94 (Kawabata's intermediate in FIG. 25B) for further conversion into the final natural products (Shibayama, H. et al., J. Am. Chem. Soc. 2021, 143, 1428-1434). We used a different reaction sequence enabled by our new strategy for access to the same intermediate 94. Key steps in our approach are sequential NHC- and boronic acid-mediated selective acylations at the C(3)- and C(6)-OH moieties. The (un-optimized) overall yield from 90 to 94 is 49%, comparable to that of Kawabata's method (35% overall yield from 90 to 94).

### Comparative Example 2

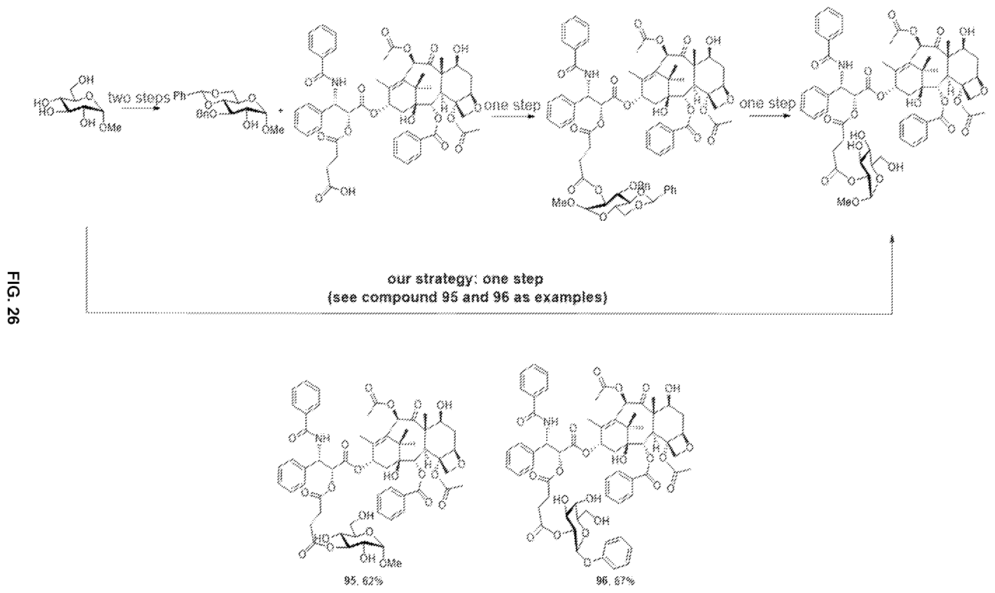

FIG. 26 is an example that concerns anticancer activities of paclitaxel (Taxol derivative). In the literature (Liu, D.-Z. et al., Bioorg. Med. Chem. Lett. 2007, 17, 617-620; Hergenrother, P. J. et al., Chem. Sci. 2013, 4, 2319-2333; and Chen, S.-T. et al., J. Med. Chem. 2008, 51, 7428-7441), conjugation of paclitaxel to sugars (via conventional methods with pre-protection and deprotection in at least 4 steps starting from glycosides) have showed enhanced activities and higher target-specificity. However, our method can be used to conjugate paclitaxel with sugar via much shorter routes (one step), more diverse sites (of the sugars) for conjugation, and a wide range of different sugars.

1. A method to selectively acylate a polyol, the method comprising the steps of:
(a) providing a mixture comprising a polyol, an acylation agent, a N-heterocyclic carbene (NHC) precursor, a base and a solvent; and
(b) subjecting the mixture to an elevated temperature for a period of time to provide a selectively acylated polyol.

2. The method according to claim 1, wherein the polyol is selected from a saccharide and a sugar alcohol.

3. The method according to claim 2, wherein the polyol is selected from the group consisting of:
glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, 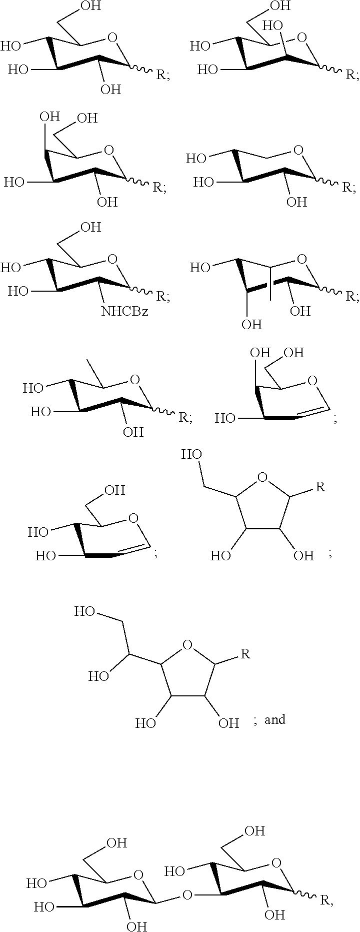
where R is any suitable moiety.

4. The method according to claim 2, wherein the polyol is selected from the group consisting of:

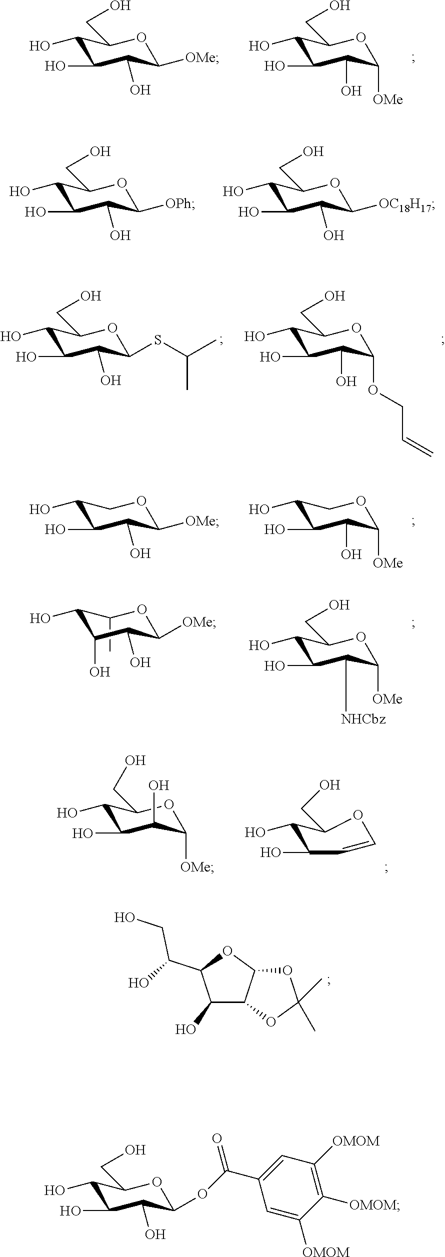

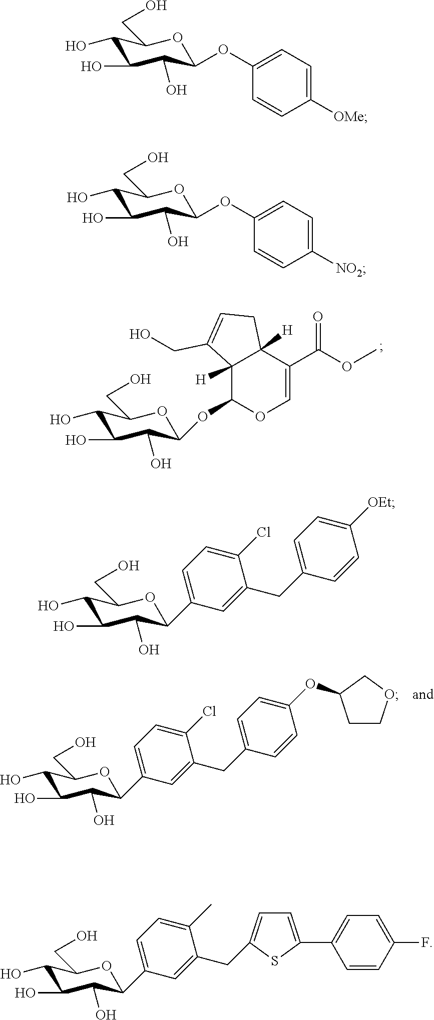

5. The method according to claim 1, wherein the acylation agent is selected from: 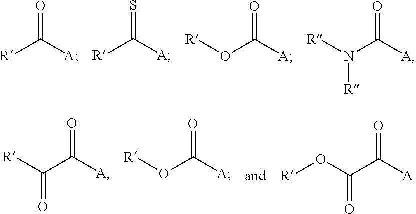
where:
A represents a moiety which forms a functional group suitable to react with a hydroxyl group to form an ester; and
R′ and R″ independently represent H or an organic moiety.

6. The method according to claim 5, wherein A represents H, OH, halo, OR^{2a}, aryl and heterocyclyl, where R^{2a} represents alkyl or aryl.

7. The method according to claim 6, wherein:
(ai) when A is H, the mixture further comprises an oxidising agent; and
(aii) when A is OH, the mixture further comprises a coupling agent.

8. The method according to claim 5, wherein the acylation agent is selected from: 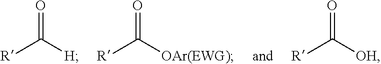
where R′ is as described in claim 5 and Ar(EWG) represents an aryl group substituted by at least one electron withdrawing group.

9. The method according to claim 5, wherein R′ is selected from:
(bi)alkyl, alkenyl, alkynyl, cycloalkyl, cycloalkenyl, which five groups are unsubstituted or substituted by one or more substituents selected from halo, nitro, CN, alkyl, alkenyl, alkynyl, cycloalkyl, aryl, heterocyclyl, OR^{3a}, S(O)ₙR^{3b}, S(O)₂N(R^{3c})(R^{3d}), N(R^{3e})S(O)₂R^{3f}, N(R^{3g})(R^{3h})
where the alkyl, alkenyl and alkynyl groups are unsubstituted or substituted by one or more substituents selected from OH, ═O, halo, alkyl and alkoxy, and
where the cycloalkyl or cycloalkenyl groups may additionally be substituted by
═O;
(bii) N(R^{3l})(R^{3m})
(biii) N(R³ⁿ)S(O)₂R^{3o}
(biv) aryl; or
(bv) heterocyclyl, where
R^{3a} to R^{3o} independently represent, at each occurrence H or C₁₋₄ alkyl, which latter group is unsubstituted or substituted by one or more substituents selected from halo,
OH and NH₂;
N is 1 or 2.

10. The method according to claim 5, wherein R″ is selected from:
(ci) alkyl, alkenyl, alkynyl, cycloalkyl, cycloalkenyl, which five groups are unsubstituted or substituted by one or more substituents selected from halo, nitro, CN, alkyl, alkenyl, alkynyl, cycloalkyl, aryl, heterocyclyl, OR^{4a}, S(O)ₙR^{4b}, S(O)₂N(R^{4c})(R^{4d}), N(R^{4e})S(O)₂R^{4f}, N(R^{4g})(R^{4h})
where the alkyl, alkenyl and alkynyl groups are unsubstituted or substituted by one or more substituents selected from OH, ═O, halo, alkyl and alkoxy, and
where the cycloalkyl or cycloalkenyl groups may additionally be substituted by ═O;
(cii) aryl; or
(ciii) heterocyclyl, where
R^{4a} to R^{4h} independently represent, at each occurrence H or C₁₋₄ alkyl, which latter group is unsubstituted or substituted by one or more substituents selected from halo, OH and NH₂;
n is 1 or 2.

11. The method according to claim 5, wherein the acylation agent is selected from: 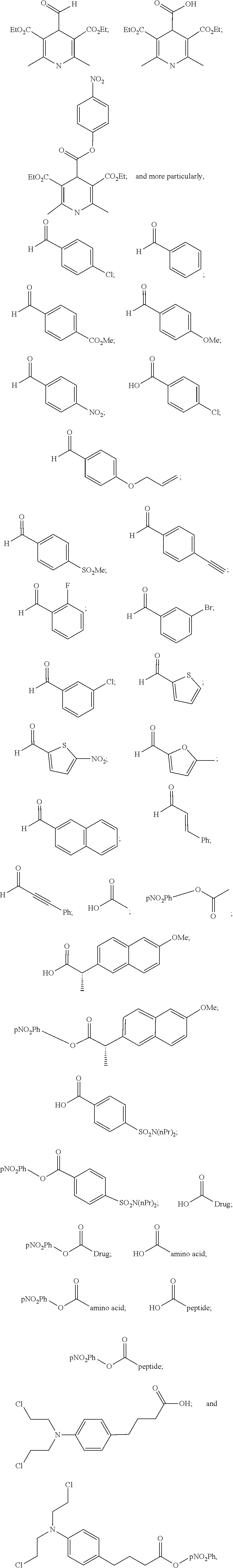
where:
Drug is any drug moiety that is linked directly to the rest of the molecule or is linked via a suitable linking moiety to the rest of the molecule;
amino acid is any amino acid; and
peptide is any peptide.

12. The method according to claim 1, wherein the NHC precursor is a pyrrolidine-based triazolium salt, a morpholine-based triazolium salt, an aminoindane-based triazolium salt, an acyclic triazolium salt, an imidazole-based heteroazolium salt, an oxazolidine-based heteroazolium salt, an imidazoline-based heteroazolium salt, or a thiazole-based heteroazolium salt.

13. The method according to claim 12, wherein the triazolium salt or heteroazolium salt is selected from:

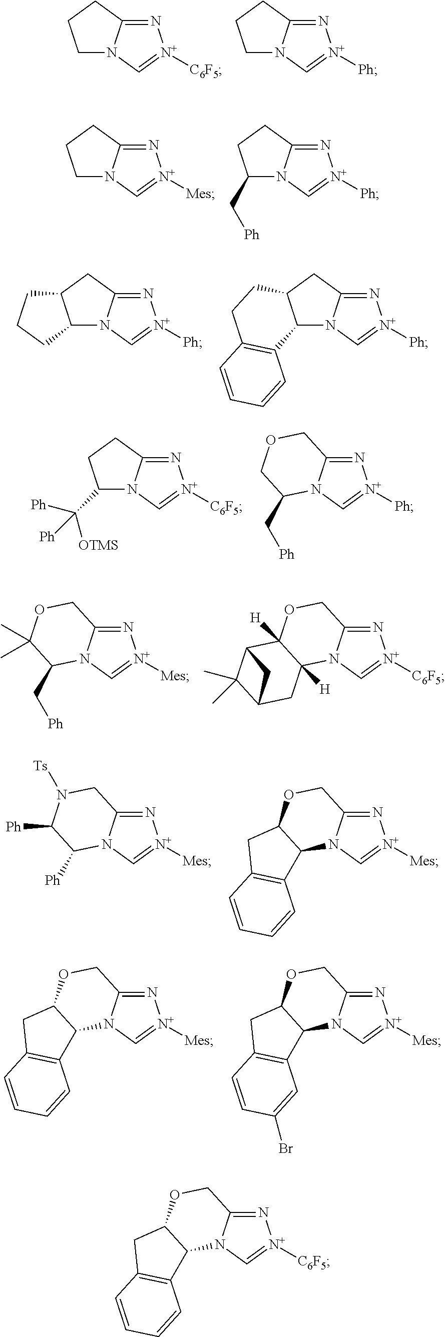

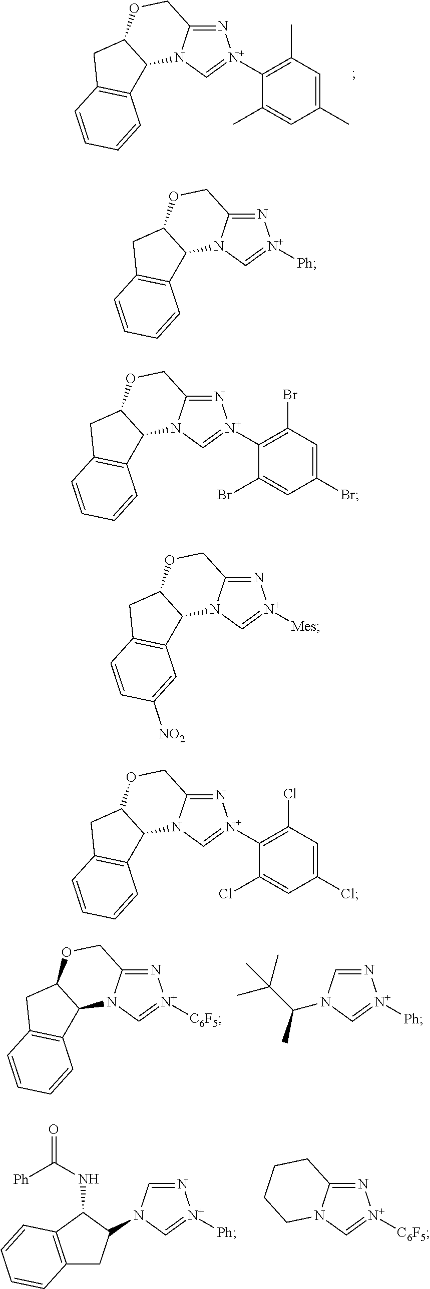

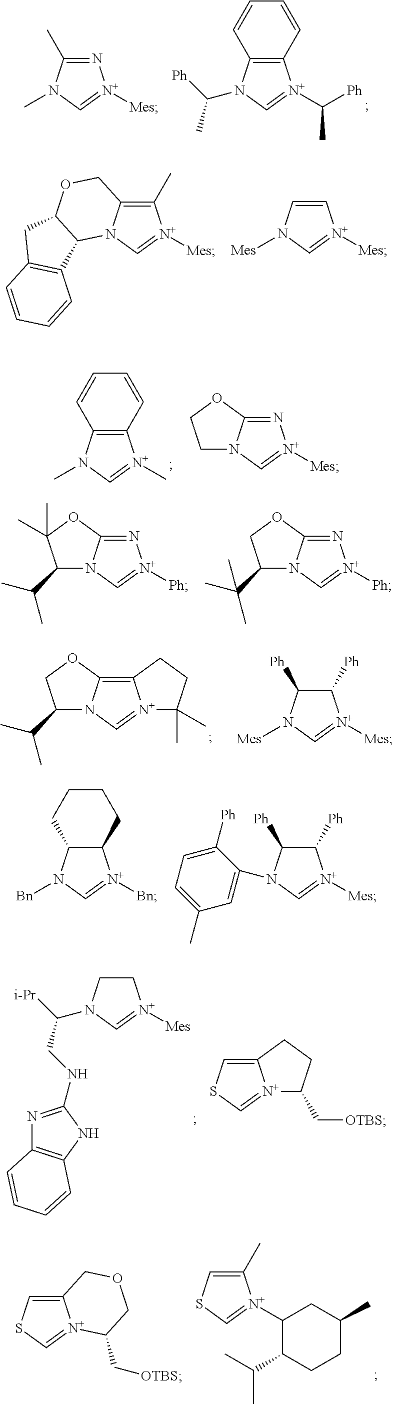

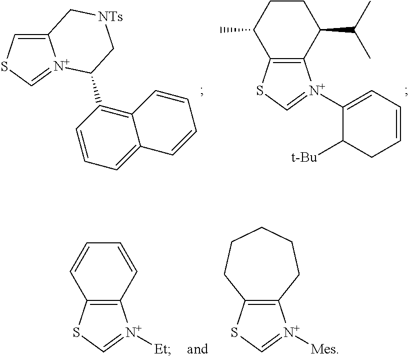

14. The method according to claim 16, wherein, when present, the boronic acid is selected from: 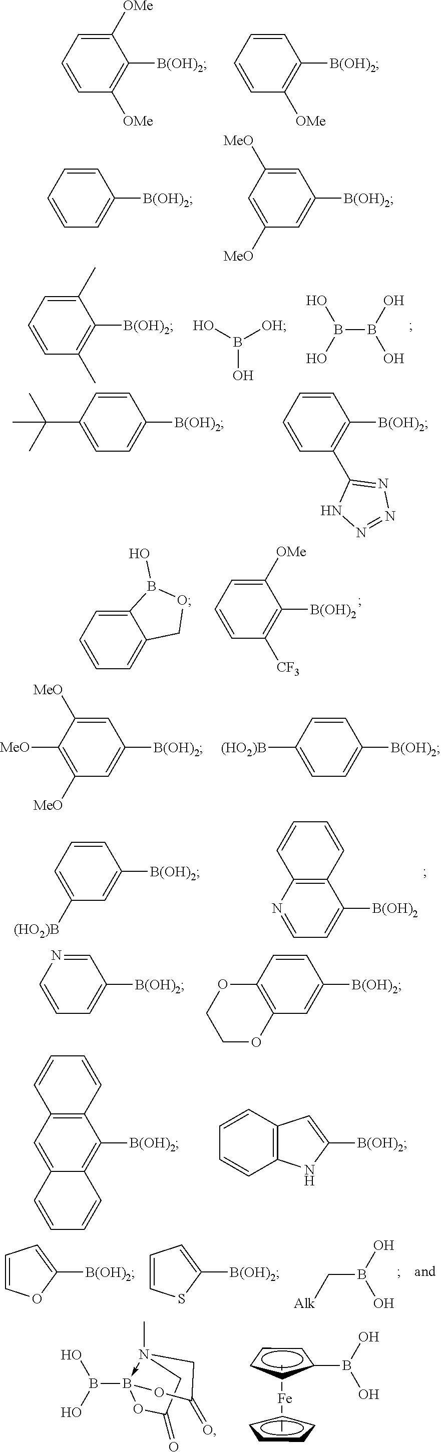
where Alk represents an alkyl group.

15. The method according to claim 1, wherein one or more of the following apply:
(di) the base is selected from DABCO, K₂CO₃, Li₂CO₃, DIPEA, DBU, NEt₃, or NaOAc; and
(dii) the solvent is selected from THF, DCM, MeCN, toluene, DMF, DMSO, EtOAc, acetone, or 1,4-dioxane.

16. The method according to claim 1, wherein the mixture further comprises a boronic acid.

17. The method according to claim 3, wherein R is selected from
(i) H;
(ii) halo;
(iii) alkyl, alkenyl, alkynyl, cycloalkyl, cycloalkenyl, which five groups are unsubstituted or substituted by one or more substituents selected from halo, nitro, CN, alkyl, alkenyl, alkynyl, cycloalkyl, aryl, heterocyclyl, OR^{1a}, S(O)ₙR^{1b}, S(O)₂N(R^{1c})(R^{1d}) N(R^{1e})S(O)₂R^{1f}, N(R^{1g})(R^{1h})
where the alkyl, alkenyl and alkynyl groups are unsubstituted or substituted by one or more substituents selected from OH, ═O, halo, alkyl and alkoxy, and
where the cycloalkyl or cycloalkenyl groups may additionally be substituted by ═O;
(iv) S(O)ₚR¹ⁱ
(v) S(O)₂N(R^{1j})(R^{1k})
(vi) OR^{1l},
(vii) N(R^{1m})(R¹ⁿ),
(viii) N(R^{1o})S(O)₂R^{1p},
(ix) aryl; or
(x) heterocyclyl, where R^{1a} to R^{1p} independently represent, at each occurrence H or C₁₋₄ alkyl, which latter group is unsubstituted or substituted by one or more substituents selected from halo, OH and NH₂;
n and p are independently 0, 1 or 2.
